SEQUENCE LISTING (18 sequences)

SEQ ID NO: 1 - DNA, 6802 bp, Homo sapiens
  source 1..6802: mol_type = other DNA
cagcagggtcacaagggcaggccgggtccttgtggagagcacatttagtgggagggacatgatttcccttcaaagtgcccattctggacgcttcccgttccatgctggacgcttcctcttccacgctggatgcttcctgttccacactggatgcttcctgttccacgctggatgtttcctgttacactctggatgcttcctgttccacactggatgcttcctgttccatcctggatgcttcctgttccatgctggacatttcctgttccactctggatgctccctgttccatgctggatgcttcctgttccatgctggatgcttcctgttccatgctggacatttcctgttccactctgcatgcttcctgttccactctggatgcttcctgttccatggtggacgtttcctgttccactctgcatgcttcctgttccatgctggatgcttccttttccattctggatgcttcctgttccatgttggatgtttcttgttccactctggatgcttcctattccattctggatgcttcctgttccatgctggacatttcctgttccatgctggatgctttctgttacatcctggatgcttcctgttccatgctggatgttttttgtttgactctggatgcttccagttccattctggatgcttcctgttccatgctggatgcttccttttccattccgcacaattcctattccattctggacacttcctgtgcgacacctccttgggttttctgtctgcccagtccctctatcctcatcccgttccctgctacctcccacctccacaatcgtccttgcccagctcctccctctctctagagcttcggcctggcaaggtccctcctgatctcagtccaggctcccccagcacaggtaggagcctagcacctgcccttggacctccccaccctgcatggtgccagcatcccccggtccccagggaggccccatttctctctctgcttgtagtccagtggccctggagtcccactgcaactcgggtgtgcccctgacctctgaggaagttaagtgtcctgtccctagccaggctatcccgtctgctcagccccagggccctgcccccaaccccttcccctcacctgcaccacaggctctggccaactctgcccaggccctgaatgggcccctctggctcccctctgctgctacactgccctgcaccacctccactcagcttcagtgtgttcatccacctgtcccaagtcccctcggcccccaggagcacagctggtggccctggctcctggcagcccatcttgttccttctggagcaccagcctcagaggccttcctgtgcagggtccacttggccagccctgggaccctcctggtctcaagcacacacgttctccctgcagccagacctgcccctgcctgtgagctcagccctgagccttggaatgccttcccatctccatcccagctcgcctttgccagctgctcagcaggatgaactcacactcccctccctgcaccatgagtcagagccagctggagacacgcccaggccaaagcagccaccagggcctagtgggggccagaagcttcagatgagaggcccaggtattgagaggctgagatcacgggcagaatggtcataatcgctgccagtatcagtccagccccagggactcagagacagagaaaagagcagcacacaaggtctgggctccccaccttctcccgtgagtacgggggagtatgggggcagccaccacccccatccccacacacccatgaggcagcctcggctgtgtctggactccccctcgccctctgacccagaaaccaccagaagaaaagggaacttcaggaagtaagtggtgccgccggtttcaatcctgttcttagtctttgcagcgtggagttcacacacctggggacctgggggccgagctgtgatttcctaggaagacaaatagcggctgacggcaggggcggggctgcccacatgtacctcgccagaacaggaagggctgagacccccacctcggtgagtggggtcagcacagggcaggggcacaggctcgggaggaggacagagcctgggggcagccgtgggcgctcctggacctgagatgctgaacaggctccaagaggctggggagacatggggtcgaggccggccccacatggaggcccaagcggagccagcacgggggaggtgggcagccttcaggcaccgatgcccacccagtgcgagacgacggggaccgtgggcaggggcttccaagccaacagggcaggacacaccagaggctgactgaggcctccaggacgaccgggctgggagcacgaggaacatgactggatgcggcagagccggccgtggggtgatgccaggatgggcacgaccgacctgagctcaggaggcagcagagcgagggaggaggagaggccccaggtgaacggaggggcttgtccaggccggcagcatcaccggagcccagggcagggtcagcagtgctggccgtggggccctcctctcagccaggaccaaggacagcaggtgagccgggagcagagcagggagggtgagtgtggcagcaggacaggagggtggaagccaaggagcccagaggcagaggcagggacaggggaggcacaggggctgggctcagagccagctgatggggttggggcacctgctggcggggagcagggctgtggtcagcagtggagaggaggggagagctgtgctgagtgcacgggcgggagaagggaagagtccagggaggcccagaaaggcccagagtgcagcaggcctggggcgaggggaagggctgaggctccgtgcgttcagggaactgacccagcagagcagaggccactgaggagctgaggttccagagaggcttccagagcaggagcagtgcagggacaggaggatccgggagctcattcaggaggggcacatgggcaagggcaaggggctctgttggggagacctgactggacactggggctgctccacagcatagggaacacgccaagtgctgcaaaatcaaaaatgagggcagaaaaacagcccaaacctggacagagggtgccaggacaggcaggggggcaacagtgacctgagtgacattgctgcccgggttgagggagggcagagtgagcagggagcaggcattggagctcagggaccaggaccaagcagccacaggtgagcagggcaggtgggggcagaaggagcagggggcacctcctggagctcaggggaccagggcagagcagcctcaggtgagcaggggctggtgggcggcaggatgagcagggggaagaccctggagctcaggggaccagggcagagcatcagaaggtgagcatggctagtgggagatgggcaagcagggggcagcccctggagctcaggggaccaggacagagcatcaggaggtgagcatggctagtgggaggtgggcgagcagggggcagcccctggaactcaggggaccagggcagagcagccgcaggtgagcacgggctggtgggaggcgggaggaacagggggcagctcctggacttcaggggaccagggagggcatctgaaggtgaacaggggccagtggggggcaggatgagcagggggaagctcctggagctcagggagccaaggcagagcagccgcaggtcagcaggggcaggtgggaagcatggggagcaggtgggcagcccctggagctcagagagccagggcagatcatccacaggagagcaggggctggtaggaagcaggaggagcaagtgggcagcttttggagctcagagcaccagggcagaagagcctcaggtgagaaggggcaggtgggaggcagaataagcaggggacagcccctggacctcaggagaccagggcagagcatcacaacgtcagcatggctggtgggaggtgggcgagcagggggcagcccctggacctcagagagccagggcagatctgcaggtgagcaggggcaggtgggaggcaggaagagcaggaggcagctcctggagctcaggggatcagggcagagcagccacaggtgagcaggggcaggtaggaagcagaaagatcaggggtcagcccctggagctcaggggacaaggggagagcatcagaaggtgagcaggactgaggctcagcctcagggagccagggcagagcagctgcaggtgagcagggccggtgggaagcaggaggagcaggtgggcagcccctggagctcagagagccagggaagatcatccgcaggtgagcaggggctggtgggaagcaggaggagcaaggggcagctcctggagctcaggggaccagggcagagcagtcgcaggtgaacaggggcaggtggggggcaggaggagcaaggagcagctcctggagctcaggggaccagggcagagcagtcgcaggtgaacaggggcaggaggagcaaggggcagctcctggagctcaggggaccagggcagagcagccgcaggtgagcaggtgcaggtggggggcaggaggagcagggggcagctcctggagctcaggggaccagggcagagcagccgcaggtgagcaggggcaggtggggtgcaggaggagcagggggcaggcactggagctcaggggaccagggcagagcagtcgcaggtgaacaggggcaggtggggggcaggagtagcaaggggcagctcctggagctcaggggaccagggcagagcagtcgcaggtgaacaggggcaggtggggggcaggaggagcagggggcagctcctggagctcaggggaccagggcagagcagccgcaggtgagcaggtgcaggtggggggcaggaggagcaggggtcaggcactggagctcaggggaccagggcagagcagccgcaggtgagcaggggcaggtggggggcaggaggagcagggggcaggcactggagctcaggggaccagggcagagcagccgcaggtgagcaggggcaggtggggggcaggaggagcagggggcaggcactggagctcaggggaccagggcagagcagccgcaggtgagcaggggcaggtggggggcaggaggagcagggggcaggcactggagctcaggggaccagggcagagcagccgcaggtcagcagggccggtgggaggcaggacgagcaggggacaggcactagagctcagggcaaggcagccacaggtgagcagggctggtgggaggcatcactcagctcctagactttggcaggagctgggtagttgctggcaacagacagctgagggctggtgaaagtgcagtgcagcctcctggtgccgggaagggagtgtgagtccatcccactgagcagttggcaagggcgagctgggatggagaagggaaggcgttccagggctcagggctgagctctcaggcaggggcaggtgtggctgcagggggaacgtgtgcttgagaccaggagggtcccacggctggtcccagcggaccctgggcaggaaggcctctgaggctggcgccccagaaggagcaagatgggctgccaggagccagggccaccagcacaatgaagctgagtggaggtggtgcagggcagtgtagcagcagagggcagccagaggggcccattcagggcctgggcagagtcagccagagcctgtggtgcaggtgaggggaaggggtggtgagcggggccctggggctgagcagaggggatggcctggctgagggcagggcgcttagcctcctcagaggtcaggggcacaccccacctgcagtgggactccagggccactgggccagcggcagagagaaatggggcctccctgtggcctgggggtcctggcaccatgcagggtggggagggccaagggcaggtgcaaggctcctacctgtgctggggggcctgggttgagcccagcagggaccttgccgggggaagctctggagagagggaggaggtgggctggtggccgagaaggccaggccagggctgggagggtgacggtgtggtgactgagcctccagaagtaatgcaggacactgggaggcagggggcatccaggcactcagggccctgacctgggctgctgcacactggggctaaggggaaaggaggggagaggctgaggaggaggctccaggaggctattccaaggcagggggttccggggccctggggctgaagggcgccgaccctatgcagtgtctggcccctctgctgcacagaagaaaagggccttggagggcagagggcaggctatgaccagggccctgggcaagtcaggccaactcactaggggagggccacgctggggcggcagggtcaggggcttcagggggctcgggggacccacgagaagccatctgagaacagtgtccactggtcaagccaggcacccataaaaggctggagtggggccaatgggcatgagccgtccctgaggtggcaccgatggccagagctgaggccaagctagaggccctggactgtgctgactcccggcagacacagagcgctgacctggctgccgagccccgcctcctaggctgcaggggtgcctgcagaagggcaccacagggccaccggtcctgcaagctttctggggcaggccgggcctgaccttggctttggggcagggggtgggctaaggtgacgcaggtggcgccagccaggcgcacacccaatgcccgtgagcccagacactggacgctgaacctcgcggacagttaagaacccaggggcctctgcgccctgggcccagctctgtcccacaccgcggtcacatggcaccacctctcttgcag

SEQ ID NO: 2 - DNA, 9483 bp, Bos taurus
  source 1..9483: mol_type = other DNA
gatcaaacactgtcaaaccaacacaagagagccccagtaccagatgtgaggctccccccagaccccagtcacagtgcagcctgtagagggaacaggcccagtcgggagaccaggaccgagaaagtgacccctgtcagtcagtccagctactcagtcatgtccgactctttgcaaccccacagactgcagcacgccaggcctccctgcccatcaccaactcccggagcttgctcaaactcaagtccattgagtcggcgatgccatccagccatctcatcctctgtcgtccccttctcctcctgcccccaatccctcccagcatcagagtcttttccaatgagtcaactcttcgcatgaggtggccaaagtattggagtttcagcttcaacgtcagtccttccaatgaacacccaggactgatctcctttacaatggactgattggatctcattgtagtccaagggattctcaagagtcttttccaacagcacagttcaaaagcattgattttttactcagctttctttatggtccaactctcacatccatacatgactactggaaaaaccatagctttgactagacggacctttgtcagcaaagtaatatctctgctttctaatgtgctgtctacgttggtcatagcttttcttccaaggaacaagtgtcttttaatttcatggctgcagtcaccatctggagtgattttggagcccaagaaaatagtctgtcactgtttccattgtttcctcgtctattttgccatgaatgccatgatcttcatttttcgaatgttgagttttaagccagctttttcactctcctctttcactttcatcaagagtctctctagttcctcttcattttctgccatgagagtggtgtcatctgcatatctgaggttattgctatttctcctggtaatcttgattccagcttgtgcttcctccatccaggtatgtcacatgatgtactctgcatagaagttaaataagcagggtcacaaaatacaatcttgatgttctccttttccaaattttaaccagtccgttgttccttatctggttctgttacttcttgacctgcatataggtttctcaggaggcagttaatgtggtctggtattcccctctctttaagaatgttccagtctgttgtgatccacccattcaaaggctttagtgtagtcaataaagcagaagcagtaaaatgctgccactttctaggtggagctcacatatggataccacacccaaaccttgaccaagccagaacctcatgacaatcatgtgcagtgctataaatgatctcagctgttacacagtcactatttacaaaatgtggggcaagagagcctgaacatgccatcttggccaattttctctctcctcatgggttgtttagctttttttctttttttttttttttaattaagctgtatgagttgcttgcacattttggaagtttattctttagtggtcacgtgatttgcaaatatcttctctaattctgcaggtagtcttgttttgttgatggttttccatggtgtacaaaagccttttagcttaattaggtcccatttgtttatttttctttttatttctattactctaggagatggatggagaaggaaatggcagcccactccagtgttcttgcctggagaatcccagggacgggggagcctggtgggctgccgtctatggggttgcactgagtcggacatgactgaagtgatatagcattagcattaggagataaatacaaaaagacaccaccttgatttatgtcaaagagtgttctgcccatgtttccctctgttttatagcctcccatttcacacttttgtctttaatccattttgagtttatttttgcccatggcactagagaatttataatttcatttattaacaggtagcaatccagtttttccagaacttattcaagagactgtcttttctccatcttacactcttgcctcctgggtcatacatgaattgatcataggtgggtgggattgttcctgggctgtctatcctgtctcactgatcactgatctataattgtggttttgtgccagcaccatagtgttttgatggccgtagctttgcagtatgctctgaagtctgggagcctgatgcctccagctccacttttgtttcttgagattgctttgactatttggggtctttatgtctcccaacaaattttcaaaagttttgttctgggtctgtggaaattgccattggtcatttgataggtgttacactgaacctgtagattgctttcggtagaagtgtcattggcacaatattgattcttctgatttaagagtatcttttctaaatagacatttctccaaaaaagacatacagatggctaacaaacacatgaaaagatgctcaacatcactcattatcagagaaatgcaaatcaaaaccactatgaggtaccatttcacaccagtcagaatggctgcgatccaaaagtctacaagcaataaatgctggagagggtgtggagaaaagggaaccctcttacactgttggtgggaatgcaaactagtacagccactatggagaacagtgtggagattccttaaaaaactggaaatagaactgccttatgatccagcaaccccactgctgggcatacacactgaggaaaccagaagggaaagagacacgtgtaccccaatgttcatcgcagcactgtttataatagccaagacatggaagcaacctagatgtccatcagcagatgaatggataagaaagctgtggtacatatacacaatggagtattactcagccattaaaaagaatacatttgaatcagttctaatgaggtggatgaaactggagcctattatacagagtgaagtaagccagaaggaaaaacataaatacagtatactaacgcatatatatggaatttagaaagatggtaacaataacccggtgtacgagacagcaaaagagacactgatgtatagaacagtcttatggactctgtgggagagggagagggtgggaagatttgggagaatggcaatgaaacatgtaaaatatcatgtaggaaacgagttgccagtccaggttcgatgcacgatgctggatgcttggggctggtgcactgggacggcccagagggatggtatggggagggaggagggaggagggttcgggatggggaacacatgtatacctgtggtggattcattttgatatttggcaaaactaatacaattatgtaaagtttaaaaataaaataaaattggaagaataaaaaaaaaaaaagagtatcttttcatctgtttgtgtcaccttaatttctttcatcagtgtcttagagctttcagagcagaggaattttgcctcctagggaaggtttattcctaggtattttattctttttgatgggatggtaagtgggattctttccttaaattctctttatggtatttcattgctgccatacagaatgcaacagatttttgtgtattaattttgtatccataaattttatccaattcattcctgagctctagggttttctgtaccttctttagcattttgcatgtgttaaatcatgtcacctgcaaacagcaggtgtttcttacttggcttctggattccctgtgtccctttctctcccctgattcttacacttaggccttcactgtgctgcactgtgggcccttgctggttacctactatatacgcagtcgtgtggatgtattagcaccaaactcctaatttatccctgggatgtggtgggtcaaccaggcgcagtgcatgaggaagaggaacaccgtgacggctaggattcccactcctcccctgggtgcaagggcgccgcttaaaccaccccaccaacccacaagacatacctcagtcacatctggactcccccaggccctctgagacagaaaacactcagaagaaaagggaacttcaggaagcaagtcgcgtcaccaggtttcattcctgttcttagtcttcacagcactgggggaagggccctcacacctcctgggactggtgaccaagtcccagggagcagggctgcagacacagaacacagggaacttcaggaggcagatggagtcaccaggtttcattcctgttcttagtcttcacagcactgggggaagggccctcacgcctcctgggactggtgaccaagtcttagggagcagggctgcagaaacagcacgcagatcccaggagccacatgtgtctgtgggccagagcagggaaggggcccggagccccaggctgtggggcagccccctccatggcccccaccatgggtgagtggggaccccgaggatgagcgcacagggacaggaatgagagccctggagggagaccggcctcgggacgcccagaggggctggagcagccgagtgggcccaggggaggcagctgcccctggtcacagtgcaggacgagcgtgtggacagaacccagacagggtgcaccacaccaagagcactgctgcccagggctggagagctcagggggctgtgggccgtggaggagcaggatgggcttgggggcgggagggaagggatgtgcatgaccaggggctctggaggccggggctgggcagtcatgaggaccagccaggacggaaagagggtctgacctgaggacgcagggaagggagaggaccccagagcagtgaagtcttctggacaggccagtggagccagcgtcggtcccagggcagggccacatgcagggactgtgggtcccgactgggctggggccgagggcagcggcagacttgggggcaaggctgcagggccagccgggttctatgtccacagcaggcaccacggggctgaacccacagccccccaggacacagggggctgaccagagctcagggcctgaggaccagcagtgggagccagagagaggcaggtgacccgaaaactggaggcagcaaggggccagaggagggtgtggatggagggatagagaagagcagagagcaaaggcaggacctctgccaggaggggcccgggtggacagagctcctccaggggagagttgcctggaggccagtgggcacctggcaagtccagggatgagagctgaacccaggaaataaaggagtgaggagtggagatgggcaggggtgagggcttccagaggggccagaggggcaagaaaggctgagggggcggcccaaggacagggggcagcaaggctgcaggtgagcaggggctgggagggcaggggggcccctagagatctgggggagcaggcacagcttgtgggtgaaaaggggttaggagggcagggggcccctggtgtttgggggagcagatagagcttttgtaggtgagcagggagctgggagggcgggggagagggtggcctggtgctttggggagcaggtacagcctgtgagggtgagcaggggctgggagggcagggggtgttcctggtgtttggggtagaaggcacagcttatgggtgagcaggggctgggagggcagggggagcccccggtgctttgggggagcaggtactggttatgagggtgagcaagggctgggaggacggggggagcccccagtgctttgggggaacaggtacaggttatgagggttgagcagggggctgagagtaaggaggtgagcctagtgctttgggggagcatgtacagcctgtgaaggtgagcaagggctaggaggacagtgggatcccctggtgctttaggggagcaggtacagcttgtgagggtgagcaagggactgggaggacaaggggtgcccctggtgtttgggggagcaggtactagttatgagggtgagcaggggctgggaggactgggggagcccctagtgctttgggggagcaggtacaggttatgagggtgagcaggggctgggaggacagggggtgttcctggtgtttggggtagaaggcacagcttatgggtgagcaggggctgggaggacaggggggcccctggagatatggggaagcaggtacagcttgttggtgagcaggggctgggaggacagggggagcccctggtgctttgggggaacaggtacaggttatgagggtgagcagggggctgagagtaaggaggtgagcctagtgctttgggggagcatgtacagcctgtgaaggtgagcaagggctaggaggacagtgggatcccctggtgctttgggggagcaggtacaggttatgagggtgagcaaggggctgggaggacaaggggtgcccctggtgctttgggggaacaagtacagcctgtgacggtgagcagggagctgggagggcagggggtgcctctggtgctttgggggagcaggtacagcttgtgagtgtgagcaagggctgggagaactgggggtgtccctggtatttgggggagcaggtactggttatgagggtgagcaggggctgggaggactgggggagcccccagtgctttgggggagcaggtacaagttatgagggtgagcagggggcttagagtaagggggggtgcacctggtgctttggggagcgttacagcctctgagggtgagcatgggctgggaggacagtgggatcccctggtgctttgggggagcaggtacagcctgtgagggtgagcaggggctgggaggacagggggagcccttggtgtttgggggagcgtgtacagcctgtgagggtgagcaggggctgggaggaaagtgggagcccctggtgtttgggggagcaggtacagcctgtgagggtgagcaggggctgggaggaaagtgggagcccctggtgtttgggggagcgtgtacagcctgtgagggtgagcaggggctgggaggaaagtaggagcccctggtgtttgggggagcgtgtacagcctgtgagggtgagcacgggctgggagggcagggggagcccctggtgctttgggggagcaggtacaggttatgagggtgagcaaggggctgggagggcagggggagcccctggtgtttgggggagcatgtacagcctgtgagggtgagcaggggctgggagggcagggggcgcccctggtgttttggggagcggtacagcttgagagggtgaacaggggctgggaggacagggggagcccctgatgctttgggggagcaggtacagcttgtgagtgtgagcaagggctgggaaaactgggggtgtccctggtgtttgggggagcaggtactggttatgagggtgagcaggggctgggaggactgggggagcccgtggtgctttgggggagcaggtacaggttatgagggtgagcagggggctgagagaaaggggggtgtgcctggtgctttggggagcattacaatctgtgagggtgagcaggggctgggagggcagggggagcccctggtgctttgggagaacaggtacagcctgtgagggtgagcaggggctgggaggacagggggagcccctggtgtttgggggagtgtgtacagcctgtgagggtgagcaggagctgggagggcagggggagcccctggtgctttgggggagcaggtacaggttatgagggtgagcaggggctgggaggactgggggtgtgcctgatgctttgggggagcaggtacagcttgtgagggtgagcagggggccgggaaagcagggtgtatctcaaaaactttaggggatcaggtagtgcttctggggatgagcagaggtctgggaggacagggggtgcccttggagttggagagcaggtacagtctgtgaggtgagtagagggctgggcaggcacagggagcccctggtgctttgggggaacaggtgcaggtcgtgagggtgagcaggggctgggaggacagggagtgtccctggtgtttggggtagaaggcacagcttatgggtgagcagggcctgggaggacagggggagcccctgttgctttgggggagcaggtacagcttgtgagggtgagctgggtctgggaagacagggggagcccctggtgctttgggggagcaggtacagcttgtgagggtgagctgggtctgggaagacagggggagcccctggtgtttgggagagcagatacagcctgtgagggttagcaggggctgggaggacagggggtgcccttggagttggagagcaggtacagtctgtgaggtgagtagagggctgggtgggcagggggagcccctggtgctttgggggaacaggtaaaggttatgagggtgagcagggggctgagagtaagggcgtgtgcctgttgctttggggaagcatgtacagcctgtgagggtgagcaggagctgggagggcaggggggagcccctggtgctttgggggagcaggtacagcctgtgagggtatccagggggctgggaggacatggggtgcccctggagatttttggaagcagtaatagcttgtgggtgagcaggaagctgcgaggacaggaggtgcccctgttgctttgggtgaacaggtacaacttgggtgagcaggggctgggaggacagggagtgcccttggagatttgggggagcaggtgcagcttctggggataagcagggggctgggagttcagggtattaggggagcagacaaagatcgcgcagctgagcaggcgctggaatgtcacgcccctgccgacgacctcagttgaccatgtgtgtgctgagcacatcggtacgaaagggtccgagggtgcaaggggccatttgtgctgtgggccagatgcagggacacagggcagtgtgagccctgcagagaagatggggcccaagagcgcagctgtccagagctgagtccagagggctgagaccagtggtgtgggtgctggacgagggtgaagggctggcgtgagggagcaagggcacagggctgggagagctcaggccacagctggtgaagcagagggctgagacaaggggtgtaggccacccctcaagacaatgggggtgctgtttggtggagggtctgcatgaagaacaaagcaaggaggagcgaagacccgagaggaagggctctggggctgcagggaaggggcgccctgagggcaccgggtgggctgcacttctgggccgagggtctgcctgtcccagcagcctgtgtggctcaggagccaaagtggggccagacctgggacgctgtgcttgatagggcggtgcaaggggccaggagctacagggcgcccactggcagtcagtcgggctgcaggcccagggagctgaccaggctgggggagcaggggccatgggaagggccaggcgcccacaggagggaaggggccctgggctctgatccaggaggccacactcaccgacccaagctgtgtccagacaccccaactgcagtacagagagccgggtggccaccatgccggccggtcatcagaccctggaagcaggtggtggctgggctcggaggtgccccaggcctgggcacctgaggtcctgctggaccccgcattcacccagcctcctctctcacag

SEQ ID NO: 3 - DNA, 10298 bp, Equus ferus
  source 1..10298: mol_type = other DNA
  variation 6918..8037: note = n = a, c, t, g, unknown or other
agatatcccccaagtcatgtcacaatactaccatgttcaaagtacatgaatgctttgaaacagactggtctcagtggctgggtgttccttgggtggctcctaatggaactcagtggttgtgtggaacaaacctctagtcttggctcctgcctggatggctgggacactgcaccctggacttcctttggaggcagggaaggagtcgccctgagctaacagctggtctcaatgttcctcccagtaaggccagatggactccatcagtgttcactggtatgaccaccagcggctatatttgctgcttccttgggctacaagatgtcatatcacatagaggctctaactaaatttactcaaactgccctcaatgaaagtcaggtaggaatacccttattaaatactcaaatgtctttagtggggagggctgtctccccatcagaatggccttagatagtctaacagcatcccaaggtgggacatgtgcactcattcaaactgaacgctgcgtttttagacctgatgagtcttccaatgtgtcctctctgctaaagcacttggaaaagcaggtaaatgccttaagcgatcctgcatccaggcttgatttgcgtggttggctcccttcaggtgtggctgccctcttgaaatctgaattgcaattcctgtgtctgttactccttggaattcttctattaatcataatatgcaaactaattactctttcctttactcagtgttgtaagactggcatgcaggctagaataatggttgctcagccacttgaggtgattgattgatcttacaaccctggatgaatttccttctcataagactatgcctaagaactcatttttcaaataatcctttcaagttttgaattatcagcactgttagtgtagctgttctataattaggcacatgcagcatcatgagtcagatcaaagcataggtacaacgtttgtgtgacaatctaaagttactgaaaacttacataacctatggaatgcttcccctgtaattgtatacctctatgctcatccactatatccaactatttcctgccattgggcataactgagcacgtcaatgagataaaagcccagcactgatggccatcatggaccagggcaggcaacaatcaccatagcaccaagggacaatatttagatcatcagtgctttctataaaaacattatcgatcaaaggtgggaaaataatgaatcaatgaaagcaataagaaaagttaaaatgttgaggtatatgaggaagccatttggtttagactaattaggcctcatctagtttttctggaaagtcctgatgtgcctgtcgaatatgcattgtacatctgctgcatttacaatgtcccaaagcaagaatgatgcccttgaaattatcctgcccccttttggcatttctttatagatgagcacttcttcccagaatgcaaggattcgttactgacctactgtgtcatcttgtgatcactcacccgcaatactagcaaagcatcttgtgactgtagtaaaagagatactcctgtcatatgtgatgtacgtcccttgttccaagacggtatataaccacgctgcacacccggcttcttcacaacacttccttccttggtgaaggttattgtcccgggctatgtagtcctcaaattggctcaaataataaactcaccccaattttgattaacagattgattatagtttattgcctcaacatttcctgaccagaaattacatagatgaacgtgcttcttatgtaacggcagcgatgctcacagcctgcggagcacatgactgagggaggctgccttccgtgggagagatggcaaaactgcgtgggcgccatggagaggaggccgggccagagccgccgggagtgtgcagtggattatcatgaattcatcccggagggaccagagggcactcccttcaccaaggctgctgctgacggcgttttctgggtgtccacagcaacattcccaggaccacaggctcctccatcacctggtcctgccccatgcagaggttcacttccctcaccagaccccagggtacccatgcgactgactccatggggacagggccactgagggaccactgctgggtctctgagagcaggacctaaggagacagggctcctcctgggagtcaggcccgggcaccactcagcgtgggcaatccccatggaaagatcacagagacagagggaacacgggcgcccgcccagcccaggcgccagactccaggtggagccttccagcccctcgctgatgcgagtggagcagagaggagcgcccccgcccacagaagccggcactgcctgcaggccaccaacgcagaagcaaatcagcagaagagctgcagaggccactctctgtggggcggtccacagcacacccatgaaccgccggacacgatatgggacccgcacgtgggcgctgctctgagaaacctcaactctagaaagtaagcacgaggaccagagagcaggggaggcagagggacccagagtagaccgttgggggatgtggagtggctctggggacacccgaggccaaagggtgctgcaaatagcaacaggtgggagggccgactcccctcgtacatctcaggtcacctcctagaaggttcgctttggactttgctcttggcccacctaagcggccacagcgtacgcccctgcccaccaaccattggaaggagccaggcctgccttcacgtccagtcacctacctggcccccggcaattccagggtggggaccatggtgccgcccagggatccccgatccaaggcctaacgtgcaagagacgggatggcccaggtttgtacacggggccttgggtgtgccgaggcactgcaagtcatgagacactgagggtccgtgtccccccacagacaaaaagctgggtcctggcccacacacgggaggacaggaacagcatgctgtctctcagacagaggagacctttgggccccagtgaccaccgtggactctgtccttgtacatagacttctttctgtccccagaggacagctgtggatgggagaatatcttccctgggaccctgggtgctaccacccttcagtcagagatccagccatggatggagccagagagggatggagggaagaggcagggacccagaggaagacggctttgtacttagggggctggcctggcaggaggacaggatgaggccctgggctgagctgcgggctgtgagcaggacagcctgtgtccacaatggacgctgactagggcagggggaggtgtcctctgggctgtgggtaccagagggctcaggtgagaggcctgggtgccctggcctttggtggggccatcgggcataatccttgtgggagggagagagcaaagagaggcatgtgagcccggacttcctgcactctccccagagtcctgggcgccagaccccctccactcccacacacccacgtgtggcagcctctgcctcacgtctgtactccccccggctctctaagacagaaaccacccagaagaaaagggaacttcagggagcaagcggtgcccctgctttcagtcccgttcttagtctttgcagggtcgtggagagtgggttcccacctccgggaaccagctaccacctccgggaaccagctaccaactcctaggaagcagggcagagggcaacaagcccggggccgcgaggagccacatgtgcccggggccagagcaggggcgtggggggagctcctcggaagccctggcttccaccacctccacagcccccacccttggtgagtggggtccccacggagcagggggaagggagtggagtcagggcagtggaaacggttgcagagctacggggagcccaggcacccagggacgactccgaggggctcaagcagctgagccagcacgggggagggcaggctgcccacgagccacggtgccaaccggacgcaggcaccaggcagccatgggcagggcacgcccacgccaacagagctggacacaccagaggcccatgaaagttaaccttgaggaaattgatgaccttgaggccagaagcctgagggctgcatttggccagctgtgctgagggcaggaccagccaggagctgagctgctgcagcacggacgccaggatgcacagatgggtttgagcacagagagggggctggctcagagcaggggaggaagaggacctccaggtggcctaagggtcctgtccacgccaatgagccagctcaagccccagggcagggtcagaggtggccgccatggtgtcctaggctgtagccaggaccgagggcagcaggtgagccaggtgcagagcccaaagggccggcggatgcacccatgtgctcagaactcagactgcacagggctgggcaggagctcagtggctacaggcagacgttgggagccaggcagtgagggtccagagcaggtgctggggttgcagtgttgggagctgggtggcaaggggagctgacccagcaatggagggagcacagtcccgaggggccttccagatgggcttctgagcgccgtgaggggcacagagacggtgaagcccagctgggccaggagctcctccaggaggagaccacggcaggcaggggctctgccaggggagagttagccagagagagggtgggcatctggtggctccaggaggcaaaggccagtgatgggaaaggaatgagcccaggatggagggtgaacctgggctgagggtgctgggagaggcaggaggggcaaaggcagccccgggcagacccagcagcctggggctgagcttagggacctgagctgtgtggaggggatcgtaggacaggagggacagagggctgctgtggacctggagttccagggacaacgggaagaacagctacggatgagcaggggaaggtgggagggcacaaaaggacaggagcctacaccaagagatgcatgggggcaggcagagcagctacaggtgagcagaggccagtgggagggctggagggaaagggggctgcccaggggctctggaagggggcaggtagagcagctacaggtgagcagaggaatgggagaccaggagggacaggtcacagcccaggagccccatgggtgtggagagcagagcttggcatgtaagtaggaagcaggaagggctgaaggggcaggggctacccagcagctccacaggagcaggcagagaagctacaggtgagcaggggccggtgggagggctgcaggggcagggggctgcccaggagctccggggagcaggcagagcagctacaggtgagcaggggccgatgggagggctgcaggggcagggggctgcccaggagctccggggagcaggcagagcagctacaggtgagcaggggccgatgggagggctgcagggacctgcccaggagctctggcggagtagtcagagcagctacaggtgagcaggggccagtgggagggctggaggggcagggtcctgcccaggagttccaggggagcaggcagagcagctataggtgagcagacgctggtgagaggtctggaggggcagggggctgcccaggagctctagcgggagcaggcagagaagctataggtgagcaggggccggtgggagggctgcaggggccggggcctgccgaggagctccggcggggtagtcagggcagctataggtgagcaggggccggtgggagggctggaggggcagtgggatacccaggagctccgccagggtagtcagagcagctacaggtgagcaggggcaagcaggagggctggaggggcaggaggctgcccaggagctccggcggggtagtcagagcagctacaggtgagcaggtgccagcgggagggctggaggggcagggggctgcccaggagctctgggaggagcaggctgagaagctgtagttgagcaggggccagtgggagggctggaggggcaggcggctgcccaggagctccgggggagcaagcagagcagctataggtgagcaggggcaggtgggggggctggaggggcagggcaatgcccaggagctccggcggggtagtcagagcagctacagtggagcaggtgccagcgggagggctggaggggcagggggctgcccaggagctccgggggagcaggcagagcagctacaggtgagcaggagcaagtgggagggctggaggggcagaggactgctcaggagctccgggggagcaggcagagcagctacaggtgagcaggagcaagtgggagggctggaggggcagaggactgctcaggagctccaggggagcaggcagaacagctactggtgagcaggggtcagcgggagcgctggaggggcagggggctgcccaggagctccgggggagcaggcagagcagctataggtgagcaggggcaggtgggagggctggaggggcagggcaatgcccaggagctccggcggggtagtcagagcagctacagtggagcaggtgccagcgggagggctggaggggcagggggctgcccaggagctccgggggagcaggcagagcagctacaggtgagcaggggccggtgggagggctgcaggggcagggtcctgcccaggagctccacaggagcagacagagaagctataggtaagcagggccggtgggagagctggaggggcagggtccggcccaggagttccgggggagcaggcagagcaggtacaggtgagcaggcgctggtgagaggtctggagcggcaggtggctgcccatgagctctggggggagcaggcagagcagctataggttagcagtggccagcaggagggctggaggggcagggagcagttcaaggagctccagcggaataatcagagcagctataggtgagcatgggccagcgggagggactgggacggggcagggagctgccaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttccagggaatcttctacagggagctagatagagaaagtttataggtaaagcaggtgccggtcgggagaagttgaaggggcagggtcctgccccaggaagttcccggggggagccaggcagagcaggttacaggtgagcaggcgattgtgagaagtctggaagcggcaggtgtcttcccatgagctcttggggggagcaggcagagcagctataggttagcagtggccagcaggaggggctggaggggcagggggcagccaaggagctccagcggggtagtcagagcagctataggtgagcaggggccagcgggagggctggaggggcaggggacttccaaggagctccatgggtgcagacagagcagaaactggtgagcaggggccggtgagagatctggagttgcagggggctgcccaggagctccgggggagcaggcagagcagctactggttgcaggggcaggtgggagggctggaggggcaggggcctgcccaggatctccagggagcaggcagagcagctataggtgagcaggggcaggtgagagggctggaggggcagcgggctgcccaggagctccaggggagcaggcagagcagctactggttgcagggcaggtgggagagctggaggggcagcgtcctgcccaggagctccacaggagcagacagagaagctataggtaagcaggggccggtgggagagctgggggggcagggtcctgcccaggagttccgggggagcaggcagagcaggtacaggtgagcaggtgctggtgagaggtctggagaggcagggggctgcccacgactttaggggatgaagggcactgggcctgagttaaacagagccctcaactggtgggacctgagggggtacagggagcagggacagctggaggctcccagggctcaggtcaggctggctgggcagggaggagagttgagctggttgagttgccaggtgcctgttgagttggatggaggggggcccctgggatggaaggagtctagggctgaaggggtcgggggcatccctgagaggaccagatcggatgcagttcttgggtccaggcacaccggccctgttctggggtttgtggggacatgggacaggagggatagacattcggactcagtgtggggtggcacagcgcagtgggctcagtgcatggaagagtaagagtccccttggggcctgagcccaggctaccagcatggggagaggccaggcaaggccctgcggctgagcagacaggactcaggggaggaggggacagggtgggcctggctgttgggggtggaggctgcaccctggcccacacgggggcctggggaggctggcaccgagaggagaggagtgccgagcgcagcgcatggctcccgcccgcgcccaggggccggctgtgctggggaaggaccacagctggaggagggctctgcagtgagcaaggagctggacaggcccaagagcagggagggcagagggcaggcggcgctggcagggaggggccggaggtgagctcaggaagccgggccaggaggacctgagcgggtagggggcccagaggcacatgcaggaggctgggctggggacacacagggatgggaggggagggggacaagaagactcccagggatggggtccagtacacggatggagtccagggaagggacggggtccaggacagggaagggatgggttcaggacagggatggggtcgagggcaaggacaaagcccaaggcatgaacagggttcaagacaggatcaggatcaggggagcgttggggacaagtactcctggtgctgtggctgtggagctgactcagatgacacgtggcctggcgagcagagacaggccatggcaaagtcactgagctgaggggccgggagacaacagggacagtgcggcgcagaggcccaccacacctgaagagatgcccaatcgcccagggctgggccaggctggggccacaggacagcagattagggtccatcagaagccgaccagaagcacaggtctctggtccgggctcagactggggactcagtcaggcgggccacctgcaggaggcctgagcagggacaaggggcaggggccccagggggctctaatctaggttctctgagcctgtggctgcagaggcaccagccctgatgacaggcagccacaaggggcatctaggcctcagactctctggaaacacacgggcgcaggggcaggccctggtggtcacacgcctcctctcttgcag

SEQ ID NO: 4 - DNA, 9313 bp, Sus scrofa
  source 1..9313: mol_type = other DNA
ggtatatatacacactagaataccactcggccgtaaaaacgaaccaaataatgccattggcagcaacacggatggaaccagagactctcatactaagtgaagtaagtcagaaagaccaataccatacgctatcacgtacatctggaatctaatatatggcacaaataaacctttccacagaaaagaaacccatggacttggagaaaatacttgtggtcgccaagggtgaggtagtggggtagactgggagtttggggttagtagatgcaacatattgcatttggcatagataaactatgagatcctgctggagagcacagggagctgtatctagtcccttgtgatggaacatgttggaggataaagtgagaaaaagaacatataaatatatacaccacacgcacacacacacacatatatgaatgactgggtcactttgccgtagagtagaaattgacagaacacagtaaatcaactataacggaaaaaaataaaaaacattaaaaaaaaaaaaacagatgctcatctccatggattctgaacacatttttgacagaattcaacacccaactttttcttgatgatttttattttttccactgcaggtggtttaccctgctctgttgattccactgtacagaaaagtgacaaagtcacacacatatatatatgtagacattctttttctcacattatcctccatcatgccccatcataggtgactagatagagggaaactacaccaacatactaaaagccatatatgacaaacccacaactatcatcattctcaatggtgaaaacctgaaagcatttccgctaagatcaggaacaagacaaggatgtctgctctcaccactctccttccacggagttttggaagtcctagccagggcaattagagaagaaaaggaatccaaatcggaaacggagaagtaacactaccgctctttacaggtgacgtgctgcgatacctagagaatcctcaaggcactaccagaaacctcttagagctcgccaatgaatttggtcaagctgcaggatacaaaattaacacacagaaattggctgccttgctctacgttaacaatgaaagagcagcgagagaaactagggaaaccatcccactgagcagtgcctcgaaatgaatgaaatgcccaggaataaacctacccaaagacacaaagacctggactctgaaaaccataaggcactgctggaagcaaccaaagacgacacaaaccggtggaaagctagaccatgctcctgccctggaagaattaatactggcaaaagggccataccacctaaggccaccctcactctcagtgcaatccctctcaaatctccaatggcattttccacagaactagaacaaaaaacctcaaaaatttgcatggaaacacaaatacctcactgacctgggctatactacaaagctgcagtcatcaaaacggtatggcactgacactcccccccaaaaaagacatacatatcagtggaacagaacagaaagcccagcaataaacccaagcacctatgctcaatccatccatgacataggaggcaagactattcaacagagaagggagagtcacttcactaagtggtgctggggagctggacagctccatgtaaagaatgaaaccagaacactcccaaacgccaaacacaccaaaaaaaaacaaaaaacaaaaaaaaaaaaataaaaaaaccctcaaaacaaatgaaactcctaaacgtaagactggatacctataactcacagaggaaaacaggcagaacgttctttgacataaatcacagcaacatcttatttgatccacctcctggaataactacaataaagacaaaagtggagttcccgtcacggctcagtggtaaccaaatctgactaggaaccatgaggttgcgggttcaatccctggccttgatcagtgggttaaggatccggcgttgccgtcagctgtggtgtaggttgcagacgcggctcggatcccacgttgctgtggctctggtgtaggccgacagctacagctccaattcgacccctagcctgggaacctccacatgccatgggtgcggccctggaaaagacaaaaagacggggaaaaaatgaaagaaagaaagaaggaaagaaggaaaggaggaaagaagaaagaaagaaagaaagaaagaaagaaagaaagaaagaaagacagacagacaaaagtaaaccaatgagatctaattaaactcaaaagcttttgaaaagcaaaagaaaccattttaaaagagaaaaacaggagttcccgtcctggctcaggggttaacgaatctgactagcatccatgaggaggcagatttgatccctggcctcactcagcaggttaaggatccggcattgccatgagctgtgagctgtggtgtaggccagcagccgtagctccaattcaacccctagcccgggaacctccatatgccacaggtggggccctaaaaaccaaaaaagacaaaggaaaacaacccacagaatgggacaaaatctttgcaaacaatgcagcccaccaaggctcaatccccaaaatatacaaacactctacaactcaacaacaacaaaaaacaaaccaacaaacaacccaatcgataaatgggcagaagacctaaatagacatttctccaaagaagacatacagatggccagaagacacatgaaaaaatcctcaacagcactaatttttagggaaatgcaaatcaaaactgcaatgaggcaccacctcacactggtcagaatggccatcattaagagtcaactaacagcaaatgctggagagggtgaggagaagagggaacccgccttcactgttggtgggaatgcaacttggtacaagcactgtggaaaacagtatggaggaacctcagaaaactgaacgtaaaaccacgattgaacccagaaatctcacccctaggcctgtatctcgatacgactttcagtcaaaaacttacatgcacaactatggactttgcagaagggttcaaaatgggcaggacatgaaaacactcaaacgcccatcaacagacgaacggattaagatgtggttctcacacaaagtaagaactacgtctcagccgtaaaaaaggaccaagtcacgccatttgcagcaacgtggatggaactagagactctcacactgagtgaagtaagtcagaaagagaaagacagagaccacatgataccacttatatgtggaatctaaaatacggcgcgattgatcctgtggacaaaacagagacagagcatggtcatggagagcagactttgctttccagggggagggagagaagagattgacagggggtttggagctggtagatgcaactgatcacattgaaaacgagtaagtgatgggtcctactgggcagcacacggaacagtgcccggcctcttgcgttagaacgtgagagaggaaggagagtgtgaaaagaaaaagaaggtaaatgtatgtacagctgtacaagggtaattgaaggaacgttgtaaatcaactctggttttaaaaaaaaaaaaaaaaatttaaagaaagaaaacgttctccagaggcagaacgagaaggtggaacggccgacaggtctctgtcctgcagcggccgctgacacgaccccaggacagcggaaatgagacccaggctgcctgtcgggcacacggcggccgccccggcaccctggtgatcctgtgcagcacgaggtgcccagtagaggggtgggcagggcagggcagggcagggcaggacctggggacgcggccgcagcaggtgggctccagccaggaaggagccacgagtggggtggggtctgctcgctgggctggaggcagggagggcacctcggctccaacatcccagccggggacccggccagaggaaggggctgcgaggatgtcttccaagcatctctttgctcttggaaccacgtggcgaagctttctgaaagcagaccagactgcagcaccacggtttctattgtgaagggatttttccagaaggagtgggaccttgcgaacggggatgtggacacagggtggcttctgatcaagcccagggccctgtgcagccaagtctcatggtcacgccgtcccggccggaccagcctgccccccacgcggacccggggaaggcaggggtcccatgaccagcacgagaacgactgactcgccttgacctcaagccacctccaggaccaggggcagacggcccggcaccacgaggaggaggcagagccccgggcagggcccacttgccacttcacctcgccagagagccgggcagatggcggcacgcgactcttggggcctgacggccccgagcccagacgctctcggggctttggccagactcgcagggctgctgagccaagcagaggctacaaagctccacgggcttccaggcaaccgctagagacacgtgggcacagctcgcagagccggtgacccacgggccagatgggaacctccctcctgtgccccctcaaggcgtctcccggaacggtggcctcccacggcgctggacggggctcggggcaggcccgaggcagcacggcgcccacaccgtccactggccgcttggaaggattcagccaacaccaccccaggcccgctgcacagccggggtgggggctgagacgtccctcaggggtccccagtcccagcctgatgctgaagttacaggacggcccaggggcgcccaggggacagggggagctgcggccctccacgaatgaggacagagggtccctgacccccacgcacggccggcaggtctcggtccacgcagagcagtgagcggctgcccgccagcccgctctcggccgggcaccaccttcacaacccctgggaactctgtccccacgtgggcagcacgggcctgggtctgggcagcaggctgggcccagccacacacgcgggggtcacaagaggtgcccggtggggtcgggcaaccaagggctcacactaggggcctggcacccgggctgcagtaggccaggcggcataatccccgtgggacatagagaaccacgacgtcctgagttcccgcaccctccccaccgctcacccaccccacacacccacatgagccgccagggcactgtctggactcccccaggccctctgggacagaaagcaaccagaagaaaagggaacttcaggaagcagccgggccacccggtttcaatcccattcttagtgtgcagggctgtgggcaggaaggtcgtgcctccagggaccagacacccacccgaggaagcagggctgcaggcacagagcacagacacaaagagccacatgcacctggggccagagcaggaggggcccggagcccggggcagcaggggagccacctcctcggcccccacactcggtgcgtggggttcacgaagaccaggggcaggggcagcggaggggggacgaggaggggagaccccacagcagggctgcagccgacccagcccagggaacacagcctgcacgcgggtcatgtcgcccgcccagcacagggaccagggagccctggcaggggcacgcccacgccaacagaggtggtgccccagaggccagaggaggtcggtggggccgccaggcctctgcggaacatgatgggctggggtcagaggcctgagggatctgctcggccaggttgctggaggcacagggctgggcaggggctgaggacgaggccaggatggacaggtgggtctgggcggaggactcagggaagagacggacacagcgcaggggacaaagaggtcacctggggttgcagggtcctgtcccggctgccggagcccgcttgagccccagggcaggtatgaaagcagtaactgtcgggtcccggggaaggagccagggccacgggcagcaggtgagccgggagcagagcaggcagggcgggccgggctcctgtgtgcccagagctggcagcacagggactgagcctaaagctccacaggccgcaggagggctgacgggagccaggggcctgagggcaggagcgggagccaggggctgggagctgggcgccagtgcagctgagccagcaactagagggagccagggaccagacaggctccaagggtggtcgcggcgagaagagggtgagctcagaagggtcaggagcccctccgggaggggaccaggtgggacagaggctctgccagggggacgctacccagagagggtgggcacccagggctccgggaacaaaggccagccctgggaaagaaaggaagccaggagtggagctagacaaggctgagggttcgggggcaggaagaggggtggtgacactgaggcccaggagccccagggaaaggggcaggtaggctgcaggtgagcagggaaccgggagggcggaaaagctgtccttagagctgctgggaacacatagagctggtgcaggtgaggaggggttgggagggcaagggggctgaacctgaagctcctcggaactggtagagcttgtgcagatgagcaggggctgggagggcagggggcagccctgggaccacctgagagctgtagatcttgggcaggtgagcacaaactggtagggcaggggaagaacctgcagttcctgggagcaggaggagcttgtgcaggtgagcaggggctggagggcagggggctaaacctggagctattgggaagaggttaggcttgtgcaggcgagcagggggtgggagggcgagggtcagtcctgggagccctttcgagccaatagagcccaggcaggtgagcaggggcttggcgggcagggagcatccctgggatgagtttgtgcaggtgagcagggggtgggagtgtagggagcagtcctagcaactcctgggagctggtagagcttgggcaggtgaacaggggctggaagtgcagggggcacatctgggaggccctgggagcaattagcgctctggcaggtgagtaggggctcggagggcagggagcagccctgggagctccttggagcaggtagagcttgggcaggtgagcagggggtgggagggcagggggcagtcctagcaactcctgggagcttgtagagcttgggcaggtgagaacgagctggtagggcaggggaatgaacctggagttactgggtcaggaggagcatgtgcaggtgagcaggggctggagggcagggggctaaacctggagctattgggaagaggttaggcttgtgcaggcaagcagggggtgggagagcaagggtcagtcctgggagcccttgcgagccggtagagcccaggcaggtgagcaggggctgggcgggcagggagcagtcctagcaactcctgggagcaggtagagcttgtgcaggtgagcagggggtgggagggtagggagcagtcctgggaactcctgcgagcaggtagagcttgtgcaggtgggcaggggtgggagggcagggggctgaacctggagctcctcagaactggtagagcttgtgcagatgagcagtggctgggagggcagggggcagccctgggagcaatagaccttgggcaggtgagaacgagctggtagggcagtggaatgaacctggagttactgggtcaagaagagcttgtgcaggcaagcaggggctgaagggcagggggctaaacctggagctattgggaagaggttaggcttgtgcaggcgagcagggggtgggagggcaagggtcagtcctgggagcccttgcgagccggtagagcccaggcaggtgagcaggggctgggagggcagggagcatccctgggacctcttgggagcaggtagagcttgtgcaggtgagcagggggtgggagggtagggagccgtcctagcaactcctgaatctggtagagcttgcggaggtgaacaggggctgggagtgcagggggcagtcctgggagcccctgggagtaattagagcttgggcaggtgagcaggagcttggagtctagggggctaaactggagctattgggaagagtttaggcttgttcaggtgagcaaggggtgggagtagagggggcattcctaggagctcccgcgagttggtcaatctcgggcaggtaagcaggggctcagaggcctggagcagccctgggaccatctgggagcagggcgagcttgggcaggtgagccgggccaggggggatgcaggacagggcaggggcaacagagtgggggtgagctcaggggagccccaggctcgagggaggggccgatatagggctagccaggctggaaagtgggctcctggggggaaggtccccaggactgcgggggcaggggcagggctgaacagacgccatgggtcacagggatcgggccaacgggccataccctgttccagcaaagtgagtggacatgggacagagcagctggagtcctaagtgagaacagcggcacagggcagtgcacatggtcggggggtcgaggggctcatctgggcctgagctggggaagcaggcaatggctgaacagaacaggggggagaggaccggcccaaccgggcaggggcgcatggggtgctggcgccccgtgggagaggaaggccaggggcagggctcggctgcagcagtgctgccaggcaggctggcagcgggaagggcaggagcagagggagggctctggcctcagcaggcagctgggaggcccagagctgggctgcaggggccggggctctggggaaagctgcctcgggtgagctcagggccaagccaggagccccggtggaggagggaccggtgggcccagaggcccagatttggagcctgggccagggccggagggatggcaggggaggggcctcggaggagactacctgccctcgcccagggaaaggggcatgttggtgccagggaccaacgcgcacgggccttgactgcagagctgacccagaggaacggggcctggagagcaaacagcaggccaggaccaggggccaggagcagagcatgggcagtgggtggcagtgggtggcagtgtccctgccaggctggggaagcaggacggtggcctcaggggaccagcagaagctggccaggaacacaggccacccgctggtcgggccaggcacccacaggggctctgggccctggcgggctctcatccaggcggtcacccagctctgaccaggcagctgcgcccagaacctgcagctgcaatggcagcgagctgggcggctggtctgccgactttctggaagcaagtgggtgctgggcgcagcggccccgctgttctgagggcccgattcgctgcccgccccacaaggaacaaggccctggcggtcacgcagcctcctctcttccag

SEQ ID NO: 5 - DNA, 337 bp, Bos taurus
  source 1..337: mol_type = other DNA
tccactagggtggccaggcacagtcaccagagggggagggctcgggtgcaggggtgcgggagggtgggggaggcagcggtgtttgtgtcctcttgtttttctctttcttctcaagccccctgcacctcatcacctgctgaaacatccaaaatagctctaggtggctactgagtcattgcgagcacagcccaacccaggtgtcccagccaggctgctcttctgagaatcgggccccaaaaccgagacctggccaggtgggcctggggcctgggcccggggccaaagcccaggggagtcctacgggggcagtgagttccccaaggcctggagagggccc

SEQ ID NO: 6 - DNA, 246 bp, Bos taurus
  source 1..246: mol_type = other DNA
ccccaccccctaggcaggtgcgaggccctctcagtttcccccaggttactcatttggggcacactcagccttgcagggcatgcaaatggctgtttgttccacactgaaaaacatgtctaagcctctgtggttatttccagaaatagcctacgcccacgccccacctgcagccccagctctgaccctccagagtgccaggctggcctggagctcaggattcgggggaccctgcaccccctgccccag

SEQ ID NO: 7 - protein, 145 aa, Homo sapiens
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSEKEEREREWEEEMEPTAARTRVP

SEQ ID NO: 8 - protein, 123 aa, Homo sapiens
MACRCLSFLLMGTFLSVSQTVLAQLDALLVFPGQVAQLSCTLSPQHVTIRDYGVSWYQQRAGSAPRYLLYYRSEEDHHRPADIPDRFSAAKDEAHNACVLTISPVQPEDDADYYCSVGYGFSP

SEQ ID NO: 9 - DNA, 177 bp, Homo sapiens
  source 1..177: mol_type = other DNA
gtcacatctggactcccccaggccctctgagacagaaaacactcagaagaaaagggaacttcaggaagcaagtcgcgtcaccaggtttcattcctgttcttagtcttcacagcactgggggaagggccctcacgcctcctgggactggtgaccaagtcccagggagcagggctgcag

SEQ ID NO: 10 - protein, 476 aa, Bos taurus
EGESHPRVFPLVSCVSSPSDESTVALGCLARDFVPNSVSFSWKFNNSTVSSERFWTFPEVLRDGLWSASSQVVLPSSSAFQGPDDYLVCEVQHPKGGKTVGTVRVIATKAEVLSPVVSVFVPPRNSLSGDGNSKSSLICQATDFSPKQISLSWFRDGKRIVSGISEGQVETVQSSPITFRAYSMLTITERDWLSQNVYTCQVEHNKETFQKNVSSSCDVAPPSPIGVFTIPPSFADIFLTKSAKLSCLVTNLASYDGLNISWSRQNGKALETHTYFERHLNDTFSARGEASVCSEDWESGEEFTCTVAHSDLPFPEKNAVSKPKDVAMKPPSVYLLPPTREQLSLRESASVTCLVKGFAPADVFVQWLQRGEPVTKSKYVTSSPAPEPQDPSVYFVHSILTVAEEDWSKGETYTCVVGHEALPHMVTERTVDKSTEGEVSAEEEGFENLNTMASTFIVLFLLSLFYSTTVTLFKVK

SEQ ID NO: 11 - protein, 471 aa, Equus ferus
ESTKTPDLFPLVSCGPSLDESLVAVGCLARDFLPNVITFSWNYQNNTVVRSQDIKNFPSVLREGKYTASSQVLLPSGDVPLVCTVNHSNGNKKVEVRPQVLIQDESPNVTVFIPPRDAFTGPGQRTSRLVCQATGFSPKEISVSWLRDGKPVESGFTTEEVQPQNKESWPVTYKVTSMLTITESDWLNQKVFTCHVEHQQGVFQKNVSSMCSPNSPVPIKIFAIPPSFAGIFLTKSAKLSCQVTNLGTYDSLSISWTRQNGEILKTHTNISESHPNGTFSALGEATICVEDWESGDDYICTVTHTDLPFPLKQAISRPDAVAKHPPSVYVLPPTREQLSLRESASVTCLVKGFSPPDVFVQWLQKGQPLSSDKYVTSAPMPEPQAPGLYFVHSILTVSEEDWSSGETYTCVVGHEALPHVVTERTVDKSTEGEVSAEEEGFENLSAMASTFIVLFLLSLFYSTTVTLFKVK

SEQ ID NO: 12 - protein, 476 aa, Ovis aries
ESESHPKVFPLVSCVSSPSDENTVALGCLARDFVPNSVSFSWKFNNSTVSSERFWTFPEVLRDGLWSASSQVALHSSSTFQGTDGYLVCEVQHPKGGKTVGTVMVVAPKVEVLSPVVSVFVPPCNSLSGNGNSKSSLICQATDFSPKQISLSWFRDGKRIVSDISEGQVETVQSSPTTYRAYSVLTITEREWLSQSAYTCQVEHNKETFQKNASSSCDATPPSPIGVFTIPPSFADIFLTKSAKLSCLVTNLASYDGLNISWSHQNGKALETHTYFERHLNDTFSARGEASVCSEDWESGEEYTCTVAHLDLPFPEKSAISKPKDVAMKPPSVYVLPPTREQLSLRESASVTCLVKGFAPADVFVQWLQKGEPVAKSKYVTSSPAPEPQDPSAYFVHSILTVTEEDWSKGETYTCVVGHEALPHMVTERTVDKSTEGEVSAEEEGFENLNTMASTFIVLFLLSLFYSTTVTLFKVK

SEQ ID NO: 13 - protein, 477 aa, Sus scrofa
ESQSAPNLFPLVSCVSPPSDESLVALGCLARDFLPSSVTFSWNYKNSSKVSSQNIQDFPSVLRGGKYLASSRVLLPSVSIPQDPEAFLVCEVQHPSGTKSVSISGPVVEEQPPVLNIFVPTRESFSSTPQRTSKLICQASDFSPKQISMAWFRDGKRVVSGVSTGPVETLQSSPVTYRLHSMLTVTESEWLSQSVFTCQVEHKGLNYEKNASSLCTSNPNSPITVFAIAPSFAGIFLTKSAKLSCLVTGLVTRESLNISWTRQDGEVLKTSIVFSEIYANGTFGARGEASVCVEDWESGDRFTCTVTHTDLPSPLKQSVSKPRGIARHMPSVYVLPPAPEELSLQEWASVTCLVKGFSPADVFVQWLQKGEPVSADKYVTSAPVPEPEPKAPASYFVQSVLTVSAKDWSDGETYTCVVGHEALPHTVTERTVDKSTEGEVSAEEEGFENLNTMASTFIVLFLLSLFYSTTVTLFKVK

SEQ ID NO: 14 - protein, 471 aa, synthetic construct
  REGION 1..471: note = Description of Artificial Sequence: Synthetic polypeptide
  REGION 1..471: note = chimeric IgM
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVETVQSSPITFRAYSMLTITERDWLSQNVYTCQVEHNKETFQKNVSSSCDVAPPSPIGVFTIPPSFADIFLTKSAKLSCLVTNLASYDGLNISWSRQNGKALETHTYFERHLNDTFSARGEASVCSEDWESGEEFTCTVAHSDLPFPEKNAVSKPKDVAMKPPSVYLLPPTREQLSLRESASVTCLVKGFAPADVFVQWLQRGEPVTKSKYVTSSPAPEPQDPSVYFVHSILTVAEEDWSKGETYTCVVGHEALPHMVTERTVDKSTEGEVSAEEEGFENLNTMASTFIVLFLLSLFYSTTVTLFKVK

SEQ ID NO: 15 - DNA, 20 bp, synthetic construct
  misc_feature 1..20: note = Description of Artificial Sequence: Synthetic oligonucleotide
  misc_feature 1..20: note = Primer
  source 1..20: mol_type = other DNA
gaccccaaaatcagcgaaat

SEQ ID NO: 16 - DNA, 24 bp, synthetic construct
  misc_feature 1..24: note = Description of Artificial Sequence: Synthetic oligonucleotide
  misc_feature 1..24: note = Primer
  source 1..24: mol_type = other DNA
tctggttactgccagttgaatctg

SEQ ID NO: 17 - DNA, 24 bp, synthetic construct
  misc_feature 1..24: note = Description of Artificial Sequence: Synthetic oligonucleotide
  misc_feature 1..24: note = Modified ZEN probe sequence
  source 1..24: mol_type = other DNA
accccgcattacgtttggtggacc

SEQ ID NO: 18 - protein, 4 aa, Severe acute respiratory syndrome coronavirus 2
RRAR

(19) United States
(12) Patent Application Publication
(10) Pub. No.: US 2024/0374704 A1
(43) Pub. Date: Nov. 14, 2024

(54) UNGULATE-DERIVED POLYCLONAL IMMUNOGLOBULIN SPECIFIC FOR CORONAVIRUS PROTEIN AND USES THEREOF

(71) Applicant: SAB Biotherapeutics, Inc., Sioux Falls, SD (US)

(72) Inventors: Tom LUKE, Sioux Falls, SD (US); Christoph L. BAUSCH, Sioux Falls, SD (US); Eddie J. SULLIVAN, Sioux Falls, SD (US); Hua WU, Sioux Falls, SD (US); Kristi A. EGLAND, Sioux Falls, SD (US)

(73) Assignee: SAB Biotherapeutics, Inc., Sioux Falls, SD (US)

(21) Appl. No.: 18/637,145

(22) Filed: Apr. 16, 2024

Related U.S. Application Data
(63) Continuation of application No. 17/364,278, filed on Jun. 30, 2021, now Pat. No. 11,975,066.
(60) Provisional application No. 63/180,935, filed on Apr. 28, 2021.
(60) Provisional application No. 63/144,784, filed on Feb. 2, 2021.
(60) Provisional application No. 63/076,121, filed on Sep. 9, 2020.
(60) Provisional application No. 63/072,683, filed on Aug. 31, 2020.

(51) Int. Cl. A61K 39/12; A61K 39/215; A61K 39/39; A61K 45/06; C07K 16/10

(52) U.S. Cl. CPC A61K 39/12; A61K 39/215; A61K 39/39; A61K 45/06; C07K 16/1003

(57) ABSTRACT

Provided are human polyclonal immunoglobulin products for use in treating or preventing coronavirus disease. Further provided are methods for making such compositions in a transgenic ungulate, e.g. using a transchromosomic bovine (TcB) system.

### CROSS-REFERENCE TO RELATED APPLICATIONS

This application is a continuation of U.S. application Ser. No. 17/364,278, filed Jun. 30, 2021, which claims priority to U.S. Application No. 63/180,935, filed Apr. 28, 2021, U.S. Application No. 63/144,784, filed Feb. 2, 2021, U.S. Application No. 63/076,121, filed Sep. 9, 2020, and U.S. Application No. 63/072,683, filed Aug. 31, 2020, each of which is incorporated by reference herein in its entirety.

### STATEMENT REGARDING GOVERNMENT FUNDING

This invention was made with government support under Medical CBRN Defense Consortium (MCDC) Other Transaction Agreement (OTA) No. W15QKN-16-9-1002 awarded by the United States Government. The government has certain rights in the invention.

### INCORPORATION BY REFERENCE OF SEQUENCE LISTING

This application contains a Sequence Listing submitted in electronic format. The Sequence Listing is provided as a file entitled SEQUENCE_LISTING_B.xml, created on May 8, 2024 and is 59,499 bytes in size. The information in electronic format of the Sequence Listing is incorporated by reference in its entirety.

### TECHNICAL FIELD

The invention relates to polyclonal immunoglobulin products for treatment of coronavirus.

### BACKGROUND

Immunoglobulin products, such as convalescent plasma, and human-derived and animal-derived immunoglobulins purified from plasma can be used to treat various infectious diseases. Treatment of COVID-19 with convalescent plasma is currently being evaluated and shows promise. Widespread use of this medical intervention would require a sufficient supply of plasma from infected patients; use of human-derived products creates a risk of donor-to-subject transmission of disease. Animal-derived immunoglobulins are known to cause severe hypersensitivity reactions and serum sickness and may be less effective because of structural differences of the antibodies that comprise human and animal immunoglobulins.

There exists, therefore, a need for immunoglobulin products for therapeutic use in patients suffering from or at risk for coronavirus disease.

### SUMMARY

The present inventors have developed a polyclonal human immunoglobulin product for treatment of coronavirus disease (e.g., COVID-19) made in ungulates (e.g., bovines) genetically engineered to produce polyclonal human immunoglobulin having a human polypeptide sequence. In addition to demonstrating that the transchromosomic bovine (TcB) system produces immunoglobulins having human polypeptide sequence, the inventors have determined that polyclonal human immunoglobulin produced according to embodiments of the methods described herein may have desirable glycosylation profiles and/or effector functions, in some cases better than analogous human-derived products.

In one aspect, provided herein is an ungulate-derived polyclonal human immunoglobulin composition, comprising a population of human immunoglobulins, wherein the population of human immunoglobulin specifically binds a coronavirus protein selected from coronavirus S protein, coronavirus M protein, coronavirus E protein, or coronavirus Nucleocapsid protein, or a combination thereof.

In some embodiments, the population of human immunoglobulin specifically binds a coronavirus S protein. In some embodiments, the population of human immunoglobulins comprise glycans covalently linked to the human immunoglobulins.

In some embodiments, the glycans comprise at least about 70%, at least about 80%, or at least about 90% N-Glycolylneuraminic acid (NGNA)-bearing glycans.

In some embodiments, 0.5 to 20% of the total immunoglobulin in the composition binds the coronavirus protein.

In another aspect, provided herein is a polyclonal human immunoglobulin composition, produced by immunizing a transgenic ungulate with a coronavirus protein or an antigenic fragment thereof, wherein the composition comprises a population of human immunoglobulins.

In some embodiments, the population of human immunoglobulins specifically binds a coronavirus protein selected from coronavirus S protein, coronavirus M protein, coronavirus E protein, or coronavirus Nucleocapsid protein or an antigenic fragment thereof. In some embodiments, the population of human immunoglobulin specifically binds a coronavirus S protein.

In some embodiments, the glycans comprise at least about 70%, at least about 80%, or at least about 90% N-Glycolylneuraminic acid (NGNA)-bearing glycans.

In some embodiments, the ungulate is a bovine.

In another aspect, provided herein is polyclonal human immunoglobulin composition, comprising a population of human immunoglobulins, wherein the population of human immunoglobulins specifically binds a coronavirus protein optionally selected from coronavirus S protein, coronavirus M protein, coronavirus E protein, or coronavirus Nucleocapsid protein. wherein the population of human immunoglobulins comprise glycans covalently linked to the human immunoglobulins, and wherein the glycans comprise at least about 70%, at least about 80%, or at least about 90% N-Glycolylneuraminic acid (NGNA)-bearing glycans.

In some embodiments, the glycans comprise at least about 70% fucosylated glycans.

In some embodiments, the glycans comprise at least about 80% fucosylated glycans.

In some embodiments, the glycans comprise at least about 90% fucosylated glycans.

In some embodiments, the glycans comprise at least about 94% fucosylated glycans.

In some embodiments, the population of human immunoglobulins binds FcγRI with a K_{D} of 15 nM or greater.

In some embodiments, the population of human immunoglobulins binds FcγRIIa with a K_{D} of 500 nM or greater.

In some embodiments, the population of human immunoglobulins binds FcγRIIb/c with a K_{D} of 1 μM or greater.

In some embodiments, the population of human immunoglobulins binds FcγRIIIa with a K_{D} of 1 μM or greater.

In some embodiments, the population of human immunoglobulins binds FcγRIIIa with a K_{D} of 1 nM or greater.

In some embodiments, the population of human immunoglobulins binds one or more human Fe gamma receptors with at most about the same affinity as a reference product.

In some embodiments, the composition is at most about as potent in a CDC assay as a reference product.

In some embodiments, the composition is at most about as potent in a ADCC assay as a reference product.

In some embodiments, the reference product is a human-derived polyclonal immunoglobulin product.

In another aspect, provided herein is method of making polyclonal human immunoglobulin for treatment of coronavirus disease, comprising administering an antigen comprising a coronavirus protein selected from coronavirus S protein, coronavirus M protein, coronavirus E protein, or coronavirus Nucleocapsid protein or an antigenic fragment thereof, or a polynucleotide encoding the antigen, to a transgenic ungulate, wherein the transgenic ungulate comprises a genome comprising a human immunoglobulin locus or an artificial chromosome comprising a human immunoglobulin locus, wherein the transgenic ungulate produces a population of human immunoglobulins that specifically binds the coronavirus S protein.

In some embodiments, administering the antigen or polynucleotide encoding the antigen 3, 4, 5, or more times.

In some embodiments, the method comprises collecting serum or plasma from the transgenic ungulate.

In some embodiments, the serum or plasma comprises a population of fully human immunoglobulins.

In some embodiments, the coronavirus is a SARS-CoV2.

In some embodiments, the antigen comprises a recombinant ectodomain of the coronavirus S protein.

In some embodiments, the recombinant ectodomain comprises one or more amino acid substitutions of a basic amino acid in the S1/S2 cleavage site (residues 682 to 685) to a non-basic residue.

In some embodiments, the recombinant ectodomain comprises one or more amino acid substitutions that stabilize the recombinant ectodomain.

In some embodiments, the one or more amino acid substitutions that stabilize the recombinant ectodomain comprise K986P and/or V986P.

In some embodiments, the method comprises administering a vaccine prime comprising a polynucleotide encoding the coronavirus S protein.

In some embodiments, the polynucleotide encoding the coronavirus S protein is a plasmid DNA molecule (pDNA).

In some embodiments, the method comprises co-administering an adjuvant comprising Montanide ISA-206 and/or Quil A at the site of vaccine prime administration.

In some embodiments, the antigen is administering in a pharmaceutical composition comprising Montanide ISA-206 and/or Quil A.

In some embodiments, the method comprises a) administering a first vaccine prime comprising the polynucleotide encoding the coronavirus S protein; b) administering a second vaccine prime comprising a polynucleotide encoding the coronavirus S protein, three to four weeks later; c) administering a first antigen comprising the recombinant ectodomain of the coronavirus S protein, four weeks later d) administering a second antigen comprising the recombinant ectodomain of the coronavirus S protein, four weeks later; and e) administering a third antigen comprising the recombinant ectodomain of the coronavirus S protein, four weeks later.

In some embodiments, the method comprises purifying the human immunoglobulin to produce an ungulate-derived polyclonal immunoglobulin composition according to the disclosure.

In another aspect, provided herein is pharmaceutical composition, comprising the composition as disclosed herein and optionally one or more pharmaceutically acceptable excipients.

In another aspect, provided herein is method of treating or preventing coronavirus disease (COVID) in a subject suffering from or at risk for COVID, comprising administering an effective amount of a composition as disclosed herein or the pharmaceutical composition as disclosed herein to the subject.

Additional embodiments, features, and advantages of the invention will be apparent from the following detailed description and through practice of the invention.

### BRIEF DESCRIPTION OF THE DRAWINGS

FIGS. 1A-1H show construction of the isHAC and isKcHACΔ vectors.

FIG. 1A shows a flow of the isHAC and isKcHACΔ vector construction. The bovinizing vector pCC1BAC-isHAC is a BAC-based one (backbone is pCC1BAC vector), consisting of 10.5 kb and 2 kb of genomic DNA as a long and short arm, respectively, 9.7 kb of the bovine genomic DNA covering the bovine I_{γ1}-S_{γ1} and its surrounding region to replace the human corresponding 6.8 kb of I_{γ1}-S_{γ1} region, the chicken 3-actin promoter-driven neo gene flanked by FRT sequence and DT-A gene. After the targeted bovinization, the neo cassette is removed by FLP introduction.

FIG. 1B shows detailed information on the targeting vector pCC1BAC-isHAC. The 2 kb of Afe I-Bam HI fragment and 10.5 kb of Apa I-Hpa I fragment for a short arm and long arm were obtained from clone h10 and clone h18/h20, respectively, derived from X, phage genomic library constructed from CHO cells containing the KHAC by screening using a probe around the human I_{γ1}-S_{γ1} region. The 9.7 kb fragment (5′ end through Bsu36 I) was obtained from clone b42 derived from the k phage bovine genomic library.

FIG. 1C shows senotyping of the bovinized I_{γ1}-S_{γ1} region. Five sets of genomic PCR were implemented, as indicated. iscont1-F1/R1 is a positive PCR specific to the homologous recombination. iscont1-F1×hIgG1-R10 is a negative PCR that is prohibited by the presence of the neo cassette. isHAC-Sw-dig-F5/R3 and isHAC-TM-dig-F3/R2 are for structural integrity check of their corresponding region, digested by Bam HI+Pvu II and Age I, Sma I or Pvu II, respectively. bNeo 5′-R×bIgG1-5′-seq-R6 is to confirm the presence of FRT sequence.

FIG. 1D shows genotyping after the FLP-FRT deletion of the neo cassette.

FIG. 1E shows extensive genomic PCR for genotyping of the isHAC vector. Location of each genomic PCR primer pair is depicted in relation to the isHAC vector structure.

FIG. 1F shows CGH analysis among three different CHO clones containing the isHAC vector. DNA from is C1-133 was used as a reference. There was no apparent structural difference of the isHAC among the three cell lines.

FIG. 1G shows extensive genomic PCR for genotyping of the isKcHACΔ vector. Location of each genomic PCR primer pair is depicted in relation to the isKcHACΔ vector structure.

FIG. 1H shows CGH analysis among three different CHO clones containing the isKcHACΔ vector. DNA from is KCDC15-8 was used as a reference. There was no apparent structural difference of the isKcHACΔ among the three cell lines.

FIG. 2A shows non-reducing gel electrophoresis characterization of a TcB-derived product compared to a sterilized solution made from human plasma (Hizentra®).

FIG. 2B shows non-reducing gel electrophoresis characterization of a TcB-derived product compared to a sterilized solution made from human plasma (Hizentra®).

FIG. 3 shows a size exclusion chromatogram of TcB-derived product.

FIG. 4 shows a complement assay comparing TcB-derived product to human-derived polyclonal immunoglobulin product.

FIG. 5A shows a bar graph of CD16A-mediated signaling in TcB-derived product compared to human-derived product.

FIG. 5B shows a bar graph of CD32A-mediated signaling in TcB-derived product compared to human-derived product.

FIG. 6A shows a plot of concentration-dependent binding of TcB-derived product or human-derived product to FcγRI (CD64).

FIG. 6B shows a plot of concentration-dependent binding of TcB-derived product or human-derived product to FcγRIIa (CD32).

FIG. 6C shows a plot of concentration-dependent binding of TcB-derived product or human-derived product to FcγRIIIa (CD16). EMABLig is a low-fucose monoclonal antibody and TG-REVO is the glycol-modified monoclonal antibody, both engineered to have increased binding to FcγRIIIa compared to human immunoglobulin.

FIG. 6D shows a plot of concentration-dependent binding of TcB-derived product or human-derived product to FcTRn.

FIG. 7A shows a pictorial representation of an IL-2 production assay.

FIG. 7B shows a plot of concentration-dependent inhibition of IL-2 production by TcB-derived immunoglobulin (“SAB-301”) or human-derived immunoglobulin (“IVIG”).

FIGS. 8A-8C. Neutralization of VSV-SARS-CoV-2 mutants by polyclonal antibody. (FIG. 8A) three lots of SAB-185 pAbs and a negative control were tested for neutralization of wild-type and (FIG. 8B) mutant VSV-SARS-CoV-2 (n=4). Error bars represent the SEM. Data are representative of four independent experiments. FIG. 8C is a heat map of the same data.

FIGS. 9A-9B. Selection of SAB-185 pAbs escape. FIG. 9A, SAB-185 pAbs were tested for neutralizing activity against VSV-SARS-CoV-2 using an MOI of 1. SAB-185 pAbs were purified from transchromosomic (Tc) bovines that were immunized with spike protein antigens, and validated by plaque reduction neutralization test (PRNT) titers against SARS-CoV-2. Data are plotted in FIG. 9A: Error bars represent the SEM. Data are representative of four independent experiments. FIG. 9B, plaque assays were performed to isolate the VSV-SARS-CoV-2-S escape mutant on Vero E6 TMPRSS2 cells in the present of the indicated pAb in the overlay. The concentration of SAB-185 pAbs added in the overlay completely inhibited viral infection (See FIGS. 9A-9B). Escape mutants are found in the control mAb experiment (shown by arrows) while no escape mutants occur in with SAB-185 pAbs (FIG. 9B). This indicates that SAB-185 as pAb is superior to mAb against escape mutants. Representative images of eight independent experiments are shown.

FIG. 10 shows selection for VSV-SARS-CoV-2 E484K escape mutation from SAB-185 vs. mAb. When tested 4 6-well plates: Escape mutants are found from mAb. No escape clone is found from SAB-185 Lot1 confirming that SAB-185 as pAb is superior to mAb against escape mutants.

FIGS. 11A-11B shows droplet digital RT-PCR (ddRT-PCR) results from mice administered high (3.75 mg) and low (1.75 mg) dose SAB-185 12 hours prior to intranasal SARS-CoV-2 challenge. Black triangles represent low dose treated animals, red squares high dose treated animals and blue circles saline treated control mice. X-axis indicates the day euthanasia and necropsy and the Y-axis RNA copy number. Lines represent geometric mean RNA copy number of the three animals in that group. (A) Data presented by group and day of euthanasia. (B) Data presented by group; ****=P<0.0001

FIGS. 12A-12B shows droplet digital RT-PCR results from mice administered high dose SAB-185 either 6 hours (red squares) or 24 hours (black triangles) after intranasal SARS-CoV-2 challenge. X-axis indicates the day euthanasia and necropsy and the Y-axis RNA copy number. Lines represent geometric mean RNA copy number of the three animals in that group. (A) Data presented by group and day of euthanasia. (B) Data presented by group; ****=P<0.0001.

FIGS. 13A-13F show examples of H&E (A, C, E) and immunohistochemical (B, D, F) staining of lung tissue from untreated K18 transgenic mice or mice given SAB-185. A and B are examples of normal lung tissue from animals given SAB-185 12 hours prior to SARS-CoV-2 intranasal challenge. C and D are from an animal euthanized at day 6 post challenge showing mild 1+ to 2+histopathological changes and immunohistochemical reactivity in the lungs. E and F are examples of lung tissue from untreated control animals showing histopathological changes and immunohistochemical staining consistent with moderate to severe pneumonia. Black arrows represent positive immunohistochemical staining (FIG. 13D and FIG. 13F); and the red arrow indicates perivascular infiltrates (FIG. 13E).

FIGS. 14A-14B show total anti-SARS-CoV-2 IgG antibody. (A) Total antibody expressed as arbitrary units (AU)/ml in animals treated with high and low dose SAB-185 12 hours prior to challenge with SARS-CoV-2. (B) total antibody expressed as AU/ml in animals treated with SAB-185 either six hours or 24 hours after intranasal challenge with SARS-CoV-2.

FIG. 15 shows Kaplan Meier plots for animals administered SAB-185 12 hours prior to or 6 hours after intranasal challenge with lethal doses of SARS-CoV-2. (A) mortality endpoints. (B) time to illness endpoint.

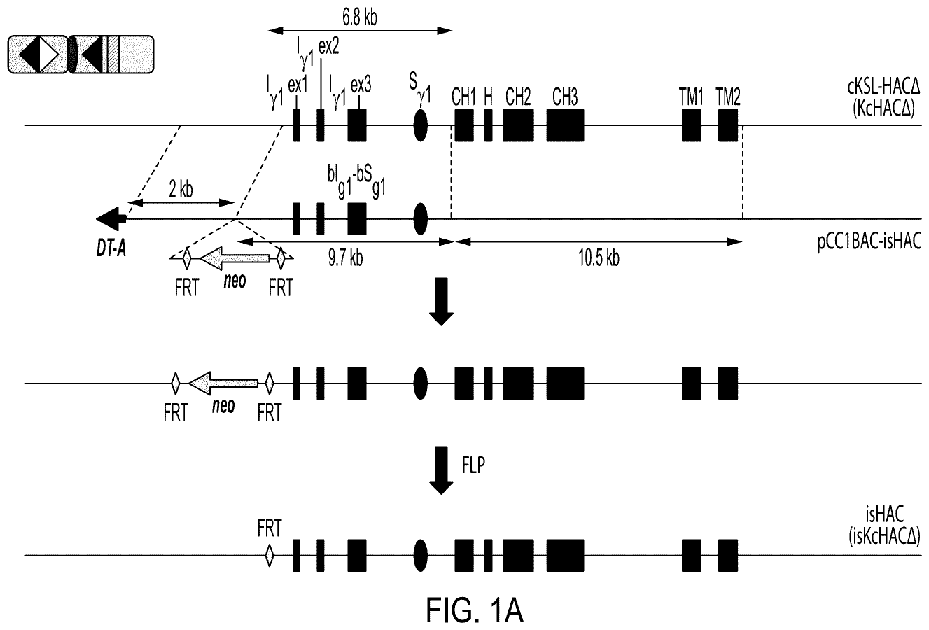

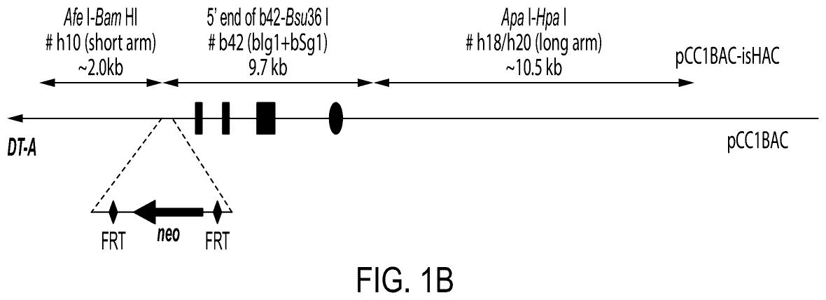

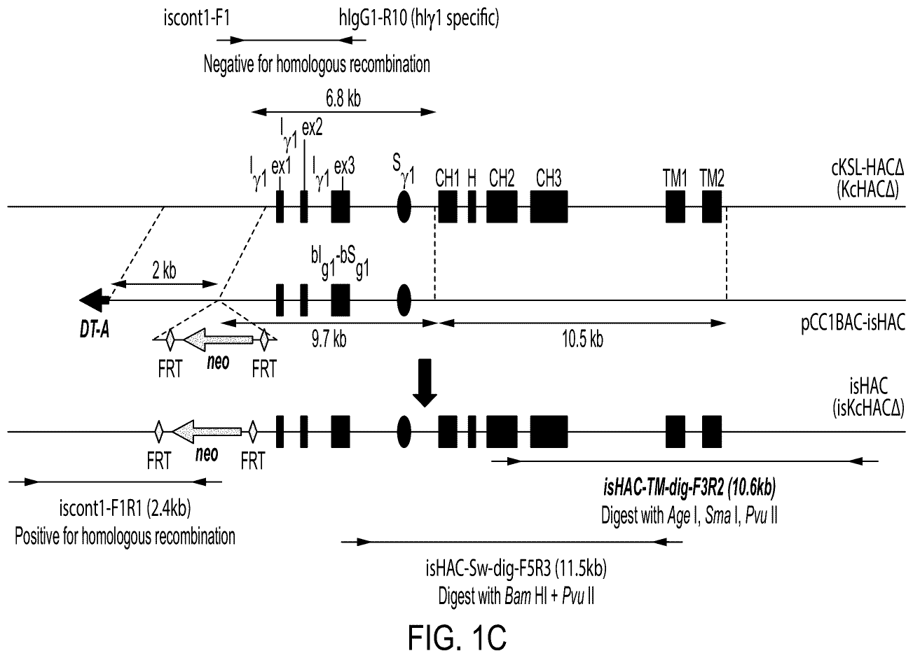

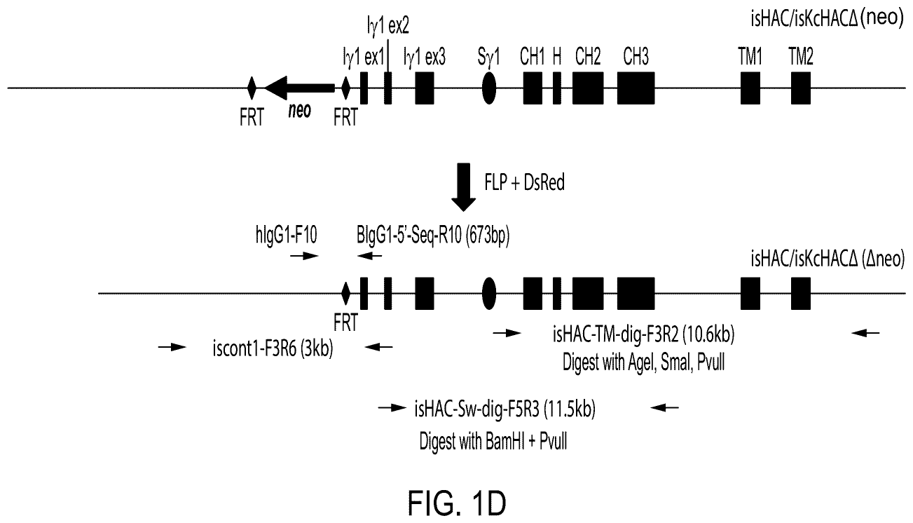

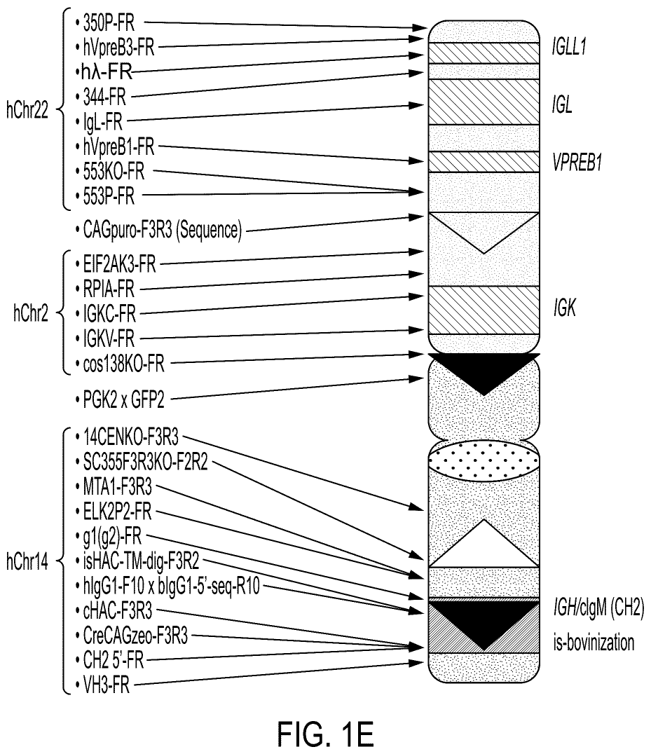

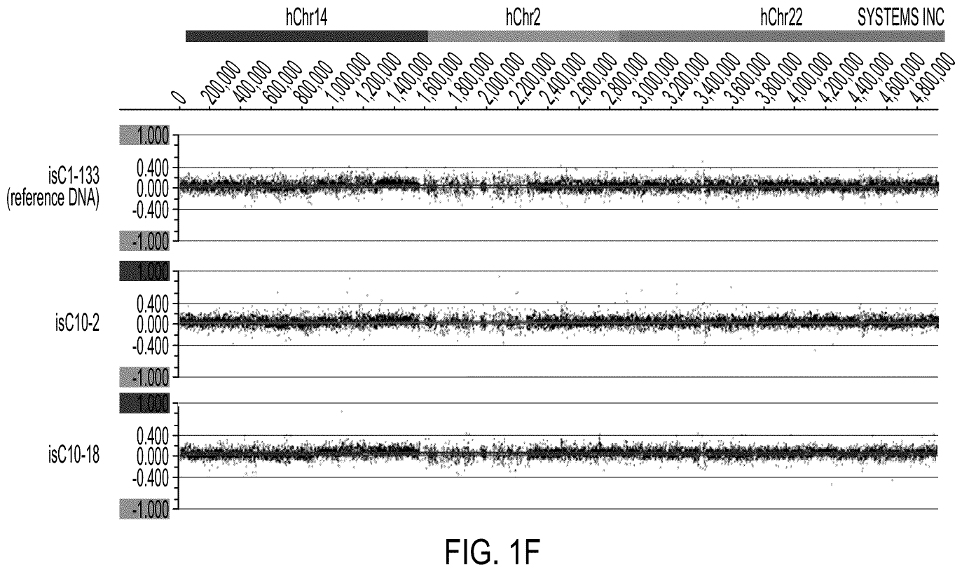

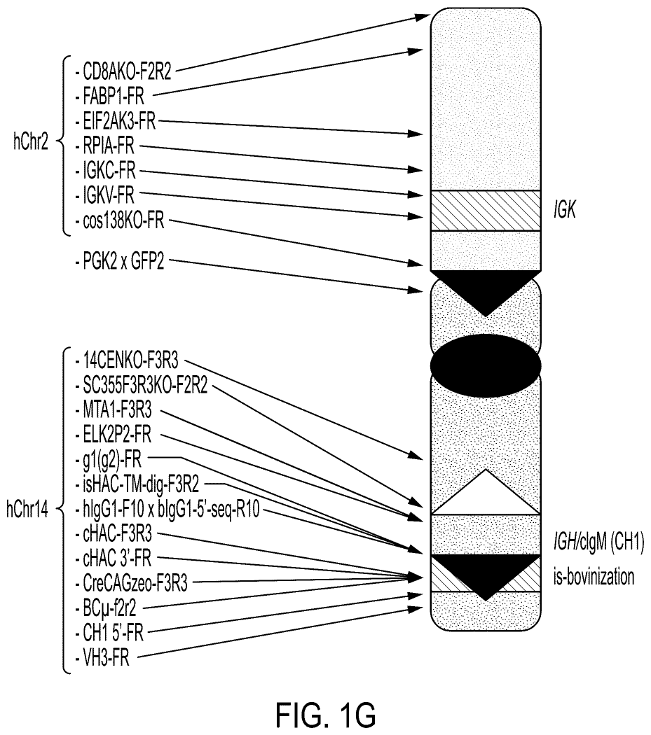

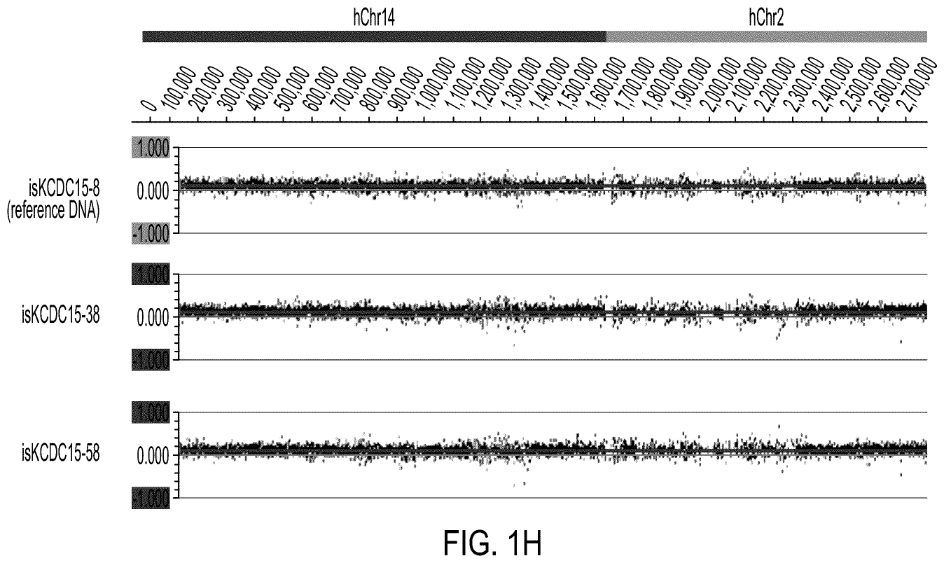

### DETAILED DESCRIPTION

The present inventors have developed a human immunoglobulin product for coronavirus disease that overcomes limitations of human- and animal-derived immunoglobulin product. Transgenic animals with the endogenous Ig locus replaced by a human artificial chromosome encoding a human Ig locus express fully human polyclonal antibodies. Immunization of such a transgenic animal with a recombinant coronavirus spike (S) protein, or an antigenic fragment thereof, and/or with a polynucleotide encoding the antigen, generates polyclonal immunoglobulin with yield, purity, and antigen specificity that enable use of this product in medical applications. Various embodiments of the invention are provided in the description that follows.

### Definitions

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a” “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” “below,” and words of similar import, when used in this application, shall refer to this application as a whole and not to any particular portions of the application.

The term “ungulate” refers to any suitable ungulate, including but not limited to bovine, pig, horse, donkey, zebra, deer, oxen, goats, sheep, and antelope.

The term “transgenic” means the cells of the ungulate comprise one or more polynucleotides encoding exogenous gene(s) (e.g. an immunoglobulin locus). Such as polynucleotide may be a portion of an artificial chromosome. Alternatively, or in addition to an artificial chromosome, one or more polypolynucleotides encoding exogenous gene(s) may be integrated into the genome of the cells of the ungulate.

The terms “polyclonal” or “polyclonal serum” or “polyclonal plasma” or “polyclonal immunoglobulin” refer to a population of immunoglobulins having shared constant regions but diverse variable regions. The term polyclonal does not, however, exclude immunoglobulins derived from a single B cell precursor or single recombination event, as may be the case when a dominant immune response is generated. A polyclonal serum or plasma contains soluble forms (e.g., IgG) of the population of immunoglobulins. The term “purified polyclonal immunoglobulin” refers to polyclonal immunoglobulin purified by serum or plasma. Methods of purifying polyclonal immunoglobulin include, without limitation, caprylic acid fractionation and adsorption with red blood cells (RBCs).

A “population” of immunoglobulins refers to immunoglobulins having diverse sequences, as opposed to a sample having multiple copies of a single immunoglobulin. Similarly stated, the term population excludes immunoglobulins secreted from a single B cell, plasma cell, or hybridoma in culture, or from a host cells transduced or transformed with recombinant polynucleotide(s) encoding a single pair of heavy and light chain immunoglobulin sequences.

The term “immunoglobulin” refers to a protein complex at least two heavy and at least two light chains in 1:1 ratio, including any of the five classes of immunoglobulin—IgM, IgG, IgA, IgD, IgE. In variations, the immunoglobulin is engineered in any of various ways known in the art or prospectively discovered, including, without limitation, mutations to change glycosylation patterns and/or to increase or decrease complement dependent cytotoxicity.

An immunoglobulin is “fully human or substantially human” when the protein sequence of the immunoglobulin is sufficiently similar to the sequence of a native human immunoglobulin that, when administered to a subject, the immunoglobulin generates an anti-immunoglobulin immune response similar to, or not significantly worse, that the immune reaction to native human immunoglobulin. A fully human immunoglobulin will comprise one or more substitutions, insertions, to deletions in variable regions, consistent with recombination, selection, and affinity maturation of the immunoglobulin sequence. In variations, the fully human or substantially human immunoglobulin is engineered in any of various ways known in the art or prospectively discovered, including, without limitation, mutations to change glycosylation patterns and/or to increase or decrease complement dependent cytotoxicity.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. Any of the compositions of the present disclosure may be isolated compositions.

The percentage of an immunoglobulin (e.g., immunoglobulin that specifically binds human coronavirus) “by mass of total immunoglobulin” refers to the concentration of a target immunoglobulin population divided by the concentration of total immunoglobulin in a sample, multiplied by 100. The concentration of target immunoglobulin can be determined by, for example, affinity purification of target immunoglobulin (e.g. on affinity column comprising coronavirus or thymocyte cell membranes) followed by concentration determination.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean plus or minus a range of up to 20%, up to 10%, or up to 5%.

The terms “immunization” and “immunizing” refer to administering a composition to a subject (e.g., a transgenic ungulate) in an amount sufficient to elicit, after one or more administering steps, a desired immune response (e.g., a polyclonal immunoglobulin response specific to coronavirus). Administration may be by intramuscular injection, intravenous injection, intraperitoneal injection, or any other suitable route. Immunization may comprise between one and ten, or more administrations (e.g. injections) of the composition, such as 1, 2, 3, 4, 5, 6, 7, 8, 9, 10, or more administrations. The first administration may elicit no detectable immune response as generally each subsequence administration will boost the immune response generated by prior administrations.

The term “target antigen” refers to any antigen use to elicit a desired immune response. The target antigen used to generate an immunoglobulin product for coronavirus disease may be recombinant spike (S) protein or an antigen fragment thereof, or nucleic acid that encodes such proteins (e.g. RNA, linear DNA, or plasmid DNA).

The term “purify” refers to separating a target cell or molecule (e.g. a population of immunoglobulins) from other substances present in a composition. Immunoglobulins may be purified by fractionation of plasma, by affinity (e.g. protein A or protein G binding, or other capture molecule), by charge (e.g. ion-exchange chromatography), by size (e.g. size exclusion chromatograph), or otherwise. Purifying a population of immunoglobulins may comprise treating a composition comprising the population of immunoglobulins with one or more of acids, bases, salts, enzymes, heat, cold, coagulation factors, or other suitable agents. Purifying may further include adsorption of a composition comprising a target cell or molecule and an impurity onto non-target cells or molecules (e.g., red blood cells) to partially or completely remove the impurity. Purifying may further include pre-treatment of serum or plasma, e.g., caprylic acid fractionation.

The terms “treating” and “treatment” refer to one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “pharmaceutically acceptable” means biologically or pharmacologically compatible for in vivo use in animals or humans, and can mean approved by a regulatory agency of the Federal or a state government or listed in the U.S. Pharmacopeia or other generally recognized pharmacopeia for use in animals, and more particularly in humans.

The term “hyperimmunized” refers to immunization regimen that generates an immune response to the subject greater than required to produce a desired titer (e.g. a binding titer) after dilution of the immunoglobulin produced by the subject. For example, if a desired titer is 1:100, one may hyperimmunize an animal by a prime immunization followed by one, two, three or more boost immunizations to produce a 1:1,000 titer, or greater titer, in the subject—so that immunoglobulin produced by the subject may be diluted in the production of a biotherapeutic in order to give a desired titer in the biotherapeutic.

An immunoglobulin is “specific to” or “specifically binds” (used interchangeably herein) to a target (e.g., coronavirus or a thymocyte antigen) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An immunoglobulin “specifically binds” to a particular protein or substance if it binds with greater affinity, avidity, more readily, and/or with greater duration than it binds to alternative particular protein or substance. For example, an immunoglobulin that specifically or preferentially binds to coronavirus S protein is an immunoglobulin that binds coronavirus S protein with greater affinity, avidity, more readily, and/or with greater duration than it binds to other proteins. An immunoglobulin that specifically binds to a first protein or substance may or may not specifically or preferentially bind to a protein cell or substance. As such, “specific binding” does not necessarily require (although it can include) exclusive binding. Generally, but not necessarily, reference to binding means specific binding.

The term “HAC vector” means a vector which comprises at least a human chromosome-derived centromere sequence, a telomere sequence, and a replication origin, and may contain any other sequences as desired for a given application. When present in a host cell, the HAC vector exists independently from a host cell chromosome in the nucleus. Any suitable methods can be used to prepare HAC vectors and to insert nucleic acids of interest into the HAC, including but not limited to those described in the examples that follow. The HAC vector is a double stranded DNA vector, as is known to those of skill in the art.

### EMBODIMENTS

Provided are methods of making a human polyclonal immunoglobulin for treatment of coronavirus disease, comprising administering an antigen comprising a coronavirus S protein or antigenic fragment thereof, or a polynucleotide encoding the antigen, to a transgenic ungulate, wherein the transgenic ungulate comprises a genome comprising a human immunoglobulin locus or an artificial chromosome comprising a human immunoglobulin locus, wherein the transgenic ungulate produces a population of human immunoglobulins that specifically binds the coronavirus S protein.

In a variation, non-human coronavirus S protein, or a polynucleotide encoding it, is used (e.g., coronavirus of a domesticated animal such as a dog, cat, sheep, etc.). The transgenic ungulate may in such cases comprise an artificial chromosome encoding an Ig locus of the non-human species such that antibodies of that species are generated.

In some embodiments, the coronavirus S protein, or a polynucleotide encoding it (that is, “the antigen”) is administered before, during, or after administration of one or more adjuvants. In some embodiments, the antigen and one or more adjuvants are administered together in a single composition, comprising optionally one or more pharmaceutically acceptable excipients.

Illustrative adjuvants include an aluminum salt adjuvant, an oil in water emulsion (e.g. an oil-in-water emulsion comprising squalene, such as MF59 or AS03), a TLR7 agonist (such as imidazoquinoline or imiquimod), or a combination thereof. Suitable aluminum salts include hydroxides (e.g. oxyhydroxides), phosphates (e.g. hydroxyphosphates, orthophosphates), (e.g. see chapters 8 & 9 of Vaccine Design. (1995) eds. Powell & Newman. ISBN: 030644867X. Plenum), or mixtures thereof. Further illustrative adjuvants include, but are not limited to, Adju-Phos, Adjumerlm, albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin Al-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, MONTANIDE™ ISA-25, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL.TM., MPL-SE, MTP-PE, MTP-PE Liposomes, Murametide, Murapalmitine, NAGO, nCT native Cholera Toxin, Non-Ionic Surfactant Vesicles, non-toxic mutant E1 12K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, ISA-25/Quil-A, Rehydragel HPA, Rehydragel LV, RIBI, Ribilike adjuvant system (MPL, TMD, CWS), S-28463, SAB-adj-1, SAB-adj-2, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes.

The immunization may be carried out by administering the antigen with, for example, a complete Freund's adjuvant or an appropriate adjuvant such as an aluminum hydroxide gel, and pertussis bacteria vaccine, subcutaneously, intravenously, or intraperitoneally into a transgenic ungulate. In one embodiment, the immunization comprises hyperimmunization. In various embodiments, the antigen is administered once to 10 times every 1 to 4 weeks after the first administration. After 1 to 14 days from each administration, blood is collected from the animal to measure the antibody value of the serum.

In some embodiments, the antigen is administered 3, 4, 5, 6 or more times. Administration of the human coronavirus may be performed, e.g., every 1-2 weeks, 2-3 weeks, 3-4 weeks, 4-5 weeks, 5-6 weeks, or 6-7 weeks, or longer intervals, e.g., every 1 week, 2 weeks, 3 weeks, 4 weeks, 5 weeks or 6 weeks. After each immunization, serum and/or plasma may be harvested from the transgenic ungulate one or more times. For example, the method may be including performing controls bleeds two or three times at intervals about 7-14 days.

In some embodiments, the antigen used to generate an immunoglobulin product for coronavirus disease may be—rather than coronavirus S protein-any other antigenic portion of the coronavirus, produced by isolation from the living virus or by recombinant methods, or nucleic acids that encoding such antigenic portions of the coronavirus (e.g. RNA, linear DNA, or plasmid DNA).

In embodiments of the methods of the disclosure, the genome of the transgenic ungulate comprises a human immunoglobulin locus. Illustrative methods are provided in U.S. Pat. Nos. 9,902,970; 9,315,824; 7,652,192; and 7,429,690; and 7,253,334, the disclosure of which are incorporated by reference herein for all purposes. Further illustrative methods are provided by Kuroiwa, Y., et al. (2009) Nat Biotechnol. 27(2):173-81, and Matsushita et al. (2015) PLoS ONE 10(6):e0130699.

The disclosure provides a human artificial chromosome (HAC) vector comprising genes encoding:
(a) one or more human antibody heavy chains, wherein each gene encoding an antibody heavy chain is operatively linked to a class switch regulatory element;
(b) one or more human antibody light chains; and
(c) one or more human antibody surrogate light chains, and/or an ungulate-derived IgM heavy chain constant region;
wherein at least one class switch regulatory element of the genes encoding the one or more human antibody heavy chains is replaced with an ungulate-derived class switch regulatory element.

The HAC vectors of the disclosure can be used, for example, for large-scale production of fully human antibodies by transgenic animals, as described for the methods of the invention. The HAC vector of the present disclosure comprises one or more genes encoding a human antibody heavy chain. Any human antibody heavy chain or combinations of human antibody heavy chains in combination may be encoded by one or more nucleic acids on the HAC. In various embodiments, 1, 2, 3, 4, 5, 6, 7, 8, or all 9 of human antibody heavy chains IgM, IgG1, IgG2, IgG3, IgG4, IgA1, IgA2, IgE and IgD may be encoded on the HAC in one or more copies. In one embodiment, the HAC comprises a human IgM antibody heavy chain encoding gene, alone or in combinations with 1, 2, 3, 4, 5, 6, 7, or the other 8 human antibody chain encoding genes. In one preferred embodiment, the HAC comprises a gene encoding at least a human IgG1 antibody heavy chain; in this embodiment, it is further preferred that the HAC comprises a gene encoding a human IgM antibody heavy chain or a gene encoding a human IgM antibody heavy chain that has been chimerized to encode an ungulate-derived IgM heavy chain constant region (such as a bovine heavy chain constant region). In another embodiment, the HAC comprises a gene encoding at least a human IgA antibody heavy chain; in this embodiment, it is further preferred that the HAC comprises a gene encoding a human IgM antibody heavy chain or a gene encoding a human IgM antibody heavy chain that has been chimerized to encode an ungulate-derived IgM heavy chain constant region (such as a bovine heavy chain constant region). In another preferred embodiment, the HAC comprises genes encoding all 9 antibody heavy chains, and more preferably where the gene encoding a human IgM antibody heavy chain has been chimerized to encode an ungulate-derived IgM heavy chain constant region. In another embodiment, the HAC may comprise a portion of human chromosome 14 that encodes the human antibody heavy chains. The variable region genes and the constant region genes of the human antibody heavy chain form a cluster and the human heavy chain locus is positioned at 14q32 on human chromosome 14. In one embodiment, the region of human chromosome 14 inserted in the HAC comprises the variable region and the constant region of the human antibody heavy chains from the 14q32 region of human chromosome 14.

In some embodiments of the HAC vectors of the present disclosure, at least one class switch regulatory element of the human antibody heavy chain encoding nucleic acid is replaced with an ungulate-derived class switch regulatory element. The class switch regulatory element refers to nucleic acid which is 5′ to an antibody heavy chain constant region. Each heavy chain constant region gene is operatively linked with (i.e. under control of) its own switch region, which is also associated with its own I-exons. Class switch regulatory elements regulate class switch recombination and determine Ig heavy chain isotype. Germline transcription of each heavy chain isotype is driven by the promoter/enhancer elements located just 5′ of the I-exons and those elements are cytokine or other activator-responsive. In a simple model of class switch, the specific activators and/or cytokines induce each heavy chain isotype germline transcription from its class switch regulatory element (i.e., activator/cytokine-responsive promoter and/or enhancer). Class switch is preceded by transcription of I-exons from each Ig heavy (IGH) locus-associated switch region. As each heavy chain constant region gene is linked with its own switch region.

Any suitable ungulate-derived class switch regulatory element can be used. For example, the human heavy chain gene isotypes listed below has the following class switch regulatory elements:
IgM: Iμ-Sμ,
IgG1: Iγ1-Sγ1,
IgG2: Iγ2-Sγ2,
IgG3: Iγ3-Sγ3,
IgG4: Iγ4-Sγ4,
IgA1: Iα1-Sα1,
IgA2: Iα2-Sα2, and
IgE: Iε-Sε.

In various embodiments, 1, more than 1, or all of the human antibody heavy chain genes on the HAC have their class switch regulatory element replaced with an ungulate-derived class switch regulatory element, including but not limited to ungulate Iμ-Sμ, Iγ-Sγ, Iα-Sα, or Iε-Sε, class switch regulatory elements. In one embodiment, an Iγ1-Sγ1 human class switch regulatory element for human IgG1 heavy chain encoding nucleic acid on the HAC (such as that in SEQ ID NO: 1) is replaced with an ungulate Iγ1-Sγ1 class switch regulatory element. Exemplary ungulate Iγ1-Sγ1 class regulatory switch elements include a bovine IgG1 Iγ1-Sγ1 class switch regulatory element (SEQ ID NO: 2), a horse Iγ1-Sγ1 class switch regulatory element (SEQ ID NO: 3), and a pig Iγ1-Sγ1 class switch regulatory element (SEQ ID: 4). However, it is not necessary to replace the human class switch regulatory element with an ungulate class switch regulatory element from the corresponding heavy chain isotype. Thus, for example, an Iγ3-Sγ3 human class switch regulatory element for human IgG3 heavy chain encoding nucleic acid on the HAC can be replaced with an ungulate Iγ1-Sγ1 class switch regulatory element. As will be apparent to those of skill in the art based on the teachings herein, any such combination can be used in the HACs of the disclosure.

In another embodiment, the HAC comprises at least one ungulate enhancer element to replace an enhancer element associated with one or more human antibody heavy chain constant region encoding nucleic acids on the HAC. There are two 3′ enhancer regions (Alpha 1 and Alpha 2) associated with human antibody heavy chain genes. Enhancer elements are 3′ to the heavy chain constant region and also help regulate class switch. Any suitable ungulate enhancer can be used, including but not limited to 3′Eα enhancers. Non-limiting examples of 3′ Eα enhancers that can be used include 3′Ea, 3′Eα1, and 3′Eα2. Exemplary 3′Eα enhancer elements from bovine that can be used in the HACs and replace the human enhancer include, but are not limited to bovine HS3 enhancer (SEQ ID NO: 5), bovine HS12 enhancer (SEQ ID NO: 6), and bovine enhancer HS4. This embodiment is particularly preferred in embodiments wherein the HAC comprises the variable region and the constant region of the human antibody heavy chains from the 14q32 region of human chromosome 14.

The HAC vectors of the present disclosure may comprise one or more genes encoding a human antibody light chain. Any suitable human antibody light chain-encoding genes can be used in the HAC vectors of the invention. The human antibody light chain includes two types of genes, i.e., the kappa/K chain gene and the lambda/L chain gene. In one embodiment, the HAC comprises genes encoding both kappa and lambda, in one or more copies. The variable region and constant region of the kappa chain are positioned at 2p11.2-2p12 of the human chromosome 2, and the lambda chain forms a cluster positioned at 22q11.2 of the human chromosome 22. Therefore, in one embodiment, the HAC vectors of the invention comprise a human chromosome 2 fragment containing the kappa chain gene cluster of the 2p11.2-2p12 region. In another embodiment, the HAC vectors of the present invention comprise a human chromosome 22 fragment containing the lambda chain gene cluster of the 22q11.2 region.

In another embodiment, the HAC vector comprises at least one gene encoding a human antibody surrogate light chain. The gene encoding a human antibody surrogate light chain refers to a gene encoding a transient antibody light chain which is associated with an antibody heavy chain produced by a gene reconstitution in the human pro-B cell to constitute the pre-B cell receptor (preBCR). Any suitable human antibody surrogate light chain encoding gene can be used, including but not limited to the VpreB1 (SEQ ID NO: 7), VpreB3 (SEQ ID NO: 8) and λ5 (also known as IgLL1, SEQ ID NO: 9) human antibody surrogate light chains, and combinations thereof. The VpreB gene and the λ5 gene are positioned within the human antibody lambda chain gene locus at 22q11.2 of the human chromosome 22. Therefore, in one embodiment the HAC may comprise the 22q11.2 region of human chromosome 22 containing the VpreB gene and the λ5 gene. The human VpreB gene of the present invention provides either or both of the VpreB1 gene (SEQ ID NO: 7) and the VpreB3 (SEQ ID NO: 8) gene and in one embodiment provides both of the VpreB1 gene and the VpreB3 gene.

In yet another embodiment, the HAC vector comprises a gene encoding an ungulate-derived IgM heavy chain constant region. In this embodiment, the IgM heavy chain constant region is expressed as a chimera with the human IgM antibody heavy chain variable region. Any suitable ungulate IgM heavy chain antibody constant region encoding nucleic acid can be used, including but not limited to bovine IgM, (SEQ ID NO: 10), horse IgM, (SEQ ID NO: 11), sheep IgM, (SEQ ID NO: 12), and pig IgM, (SEQ ID NO: 13). In one embodiment, the chimeric IgM comprises the sequence in SEQ ID NO: 14. Pre-BCR/BCR signaling through the IgM heavy chain molecule promotes proliferation and development of the B cell by interacting with the B cell membrane molecule Ig-alpha/Ig-beta to cause a signal transduction in cells. Transmembrane region and/or other constant region of IgM are considered to have important roles in the interaction with Ig-alpha/Ig-beta for signal transduction. Examples of the IgM heavy chain constant regions include nucleic acids encoding constant region domains such as CH1, CH2, CH3, and CH4, and the B-cell transmembrane and cytoplasmic domains such as TM1 and TM2. The nucleic acid encoding an ungulate-derived IgM heavy chain constant region which is comprised in the human artificial chromosome vector of the invention is not particularly limited so long as the region is in a range which may sufficiently induce the signal of the B-cell receptor or B-cell proliferation/development in the above-described IgM heavy chain constant region. In one embodiment, the nucleic acid encoding an ungulate-derived IgM heavy chain constant region provides a transmembrane and cytoplasmic TM1 domain and TM2 domain derived from an ungulate, and in other embodiments encodes the ungulate-derived CH2 domain, CH3 domain, CH4 domain, TM1 domain, and TM2 domain or the ungulate-derived CH1 domain, CH2 domain, CH3 domain, CH4 domain, TM1 domain, and TM2 domain.

In one embodiment, the gene encoding the IgM heavy chain constant region of the bovine is a gene encoding a bovine IgM heavy chain constant region which is included in an IGHM region at which a bovine endogenous IgM heavy chain gene is positioned (derived from IGHM) or a gene encoding a bovine IgM heavy chain constant region in an IGHML1 region (derived from IGHML1). In another embodiment, the gene encoding a bovine IgM heavy chain constant region is included in the IGHM region.

In a further embodiment, the HAC comprises a gene encoding a human antibody heavy chain comprises a gene encoding a human heavy chain (for example, a human IgG heavy chain, such as an IgG1 heavy chain), and wherein a transmembrane domain and an intracellular domain of a constant region of the human heavy chain gene are replaced with a transmembrane domain and an intracellular domain of an ungulate-derived heavy chain (for example, an ungulate IgG heavy chain, such as an IgG1 heavy chain), constant region gene. In one embodiment, gene encoding the transmembrane domain and the intracellular domain of an ungulate-derived (such as bovine) IgG (such as IgG1) heavy chain constant region are used to replace the corresponding regions of the human IgG heavy chain gene. In another embodiment, the gene encoding the TM1 and TM2 domains of an ungulate-derived (such as bovine) IgG (such as IgG1) heavy chain constant region are used to replace the corresponding regions of the human IgG heavy chain gene. In another embodiment, the gene encoding the one or more of the CH1-CH4 domains and/or the TM1 and TM2 domains of an ungulate-derived (such as bovine) IgG (such as IgG1) heavy chain constant region are used to replace the corresponding regions of the human IgG heavy chain gene.

The disclosure further provides transgenic ungulates comprising a HAC vector according to any embodiment or combination of embodiments of the disclosure. The transgenic ungulate comprising the HAC vector of the present invention refers to an animal into which the human artificial chromosome vector of the present invention is introduced. The transgenic ungulate having the HAC of the present invention is not particularly limited so long as the animal is a transgenic ungulate in which the human artificial chromosome fragment may be introduced into a cell thereof, and any non-human animals, for example, ungulates such as cows, horses, goats, sheep, and pigs; and the like may be used. In one aspect, the transgenic ungulate is a bovine. A transgenic ungulate having the HAC vector of the present invention may be constructed, for example, by introducing the HAC vector of the present disclosure into an oocyte of a host animal using any suitable technique, such as those described herein. The HAC vector of the present invention may, for example, be introduced into a somatic cell derived from a host ungulate by a microcell fusion method. Thereafter, the animal having the HAC vector may be constructed by transplanting a nucleus or chromatin agglomerate of the cell into an oocyte and transplanting the oocyte or an embryo to be formed from the oocyte into the uterus of a host animal to give birth. It may be confirmed by a method of Kuroiwa et al. (Kuroiwa et al., Nature Biotechnology, 18, 1086-1090, 2000 and Kuroiwa et al., Nature Biotechnology, 20, 889-894) whether an animal constructed by the above method has the human artificial chromosome vector.

The disclosure further provides transgenic ungulates comprising genes integrated into its genome encoding:
(a) one or more human antibody heavy chains, wherein each gene encoding an antibody heavy chain is operatively linked to a class switch regulatory element;
(b) one or more human antibody light chains; and
(c) one or more human antibody surrogate light chains, and/or an ungulate-derived IgM heavy chain constant region;
wherein at least one class switch regulatory element of the genes encoding the one or more human antibody heavy chains is replaced with an ungulate-derived class switch regulatory element.

In such embodiments, the transgenic ungulate may comprise any embodiment or combination of embodiments of the nucleic acids as described herein for the HAC, but rather than being present in a HAC, they are integrated into a chromosome of the ungulate.

The disclosure further provides a method of producing a human antibody, comprising: (a) administering human coronavirus, or other target antigen of the disclosure, to the transgenic ungulate of any embodiment or combination of embodiments of the disclosure to produce and accumulate a population of human immunoglobulins specific to human coronavirus (or to T cells, B cells, and/or monocytes) in the serum or plasma of the ungulate; and optionally (b) isolating, recovering, and/or purifying the population of human immunoglobulins specific to the human coronavirus (or to T cells, B cells, and/or monocytes) from the serum or plasma of the ungulate.

The polyclonal serum or plasma, or human immunoglobulin purified from the polyclonal serum or plasma, may be used as an Immunoglobulin product for coronavirus disease.

In a variation, the disclosure provides a method of recovering the protein sequence of a human antibody comprises: (i) isolating lymphocytes from the transgenic ungulate; (ii) generating a human monoclonal antibody producing hybridoma from the lymphocytes; and (iii) recovering human monoclonal antibody specific to the antigen from the hybridoma. In another embodiment, the lymphocytes from the transgenic ungulate are isolated from lymph nodes of the transgenic ungulate. In a further embodiment the transgenic ungulate is hyperimmunized with the target antigen.

A coronavirus protein-specific human immunoglobulin (such as coronavirus S protein-specific human immunoglobulin) may be produced by immunizing the transgenic ungulate having the HAC vector with human coronavirus protein, or another antigen of the disclosure, to produce the coronavirus protein-specific human immunoglobulin in the serum or plasma of the transgenic ungulate and recovering the coronavirus protein-specific human immunoglobulin from the serum or plasma of the transgenic ungulate.

Examples of methods for detecting and measuring the coronavirus S protein-specific human immunoglobulin in a composition include a binding assay by an enzyme-linked immunosorbent assay, and the like. The binding amount of a human immunoglobulin may be measured by incubating the composition comprising the human immunoglobulin with cells (e.g., coronavirus, T cells, B cells and/or monocytes, or recombinant protein antigen(s)), and then using an antibody specifically recognizing human immunoglobulin.

In a variation, the methods of the disclosure are used to generate a monoclonal antibody. Methods of preparing and utilizing various types of antibodies are well-known to those of skill in the art and would be suitable in practicing the present invention (see, for example, Harlow, et al. Antibodies: A Laboratory Manual, Cold Spring Harbor Laboratory, 1988; Kohler and Milstein, Nature 256:495 (1975)). An example of a preparation method for hybridomas comprises the following steps of: (1) immunizing a transgenic ungulate with coronavirus; (2) collecting antibody-producing cells from the transgenic ungulate (i.e. from lymph nodes); (3) fusing the antibody-producing cells with myeloma cells; (4) selecting hybridomas that produce a monoclonal antibody specific to coronavirus from the fused cells obtained in the above step; and optionally (5) selecting a hybridoma that produces a monoclonal antibody specific to coronavirus from the selected hybridomas.

In embodiments of the methods of producing polyclonal immunoglobulin specific for coronavirus protein (such as coronavirus S protein-specific human immunoglobulin), the transgenic ungulate produces human polyclonal immunoglobulin specific for coronavirus protein. The method may comprise collecting the polyclonal serum and/or polyclonal plasma from the transgenic ungulate. In some embodiments, the ungulate is a bovine. In some embodiments, the polyclonal immunoglobulin composition comprises a population of fully human immunoglobulins. In some embodiments, the polyclonal immunoglobulin composition comprises a population of fully human immunoglobulins, substantially human immunoglobulins.

Some embodiments of the methods of the disclosure, and related compositions, have the surprising advantage that the coronavirus protein-specific immunoglobulins (such as coronavirus S protein-specific human immunoglobulin) are produced in high yield, in high purity, and/or as a high percentage of total immunoglobulin present in the serum or plasma of the transgenic ungulate. Furthermore, some embodiments produce coronavirus protein-specific immunoglobulins having glycans that comprise at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95% or higher percentage of fucosylated glcyans. Furthermore, some embodiments produce coronavirus protein-specific immunoglobulins having at most about the same ADCC or CDC activity as a reference immunoglobulin preparation, e.g. human-derived immunoglobulin.

In some embodiments, the population of human immunoglobulins binds FcγRI with a K_{D} of 15 nM or greater. In some embodiments, the population of human immunoglobulins binds FcγRIIa with a K_{D} of 500 nM or greater. In some embodiments, the population of human immunoglobulins binds FcγRIIb/c with a K_{D} of 1 μM or greater. In some embodiments, the population of human immunoglobulins binds FcγRIIIa with a K_{D} of 1 μM or greater. In some embodiments, the population of human immunoglobulins binds FcγRIIIa with a K_{D} of 1 nM or greater.

In some embodiments of the methods and compositions of the disclosure, the polyclonal serum or polyclonal plasma comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-0.5%, 0.5-1%, 1-1.5%, 1.5-2%, 2-2.5%, 2.5-3%, 3-3.5%, 3.5-4% 4-4.5%, 4.5-5%, 5-5.5%, 5.5-6%, 6-6.5%, 6.5-7%, 7-7.5% 7.5-8%, 8-8.5%, 8.5-9%, 9-9.5% 9.5-10% or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-1%, 1-2%, 2-3%, 3-4%, 4-5%, 5-6%, 6-7%, 7-8%, 8-9%, 9-10%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal serum or polyclonal plasma.

In some embodiments of the methods and compositions of the disclosure, the polyclonal serum or polyclonal plasma comprises 1-4%, 2-5%, 3-6%, 4-7%, 5-8%, 6-9%, or 7-10% fully human (or substantially human) immunoglobulin by mass of total immunoglobulin in the polyclonal serum or polyclonal plasma.

In some embodiments of the methods and compositions of the disclosure, the polyclonal immunoglobulin comprises at least 0.1%, at least 0.2%, at least 0.3%, at least 0.4%, at least 0.5%, at least 0.6%, at least 0.7%, at least 0.8%, at least 0.9%, at least 1%, at least 1.1%, at least 1.2%, at least 1.3%, at least 1.4%, at least 1.5%, at least 1.6%, at least 1.7%, at least 1.8%, at least 1.9%, at least 2%, at least 2.1%, at least 2.2%, at least 2.3%, at least 2.4%, at least 2.5%, at least 2.6%, at least 2.7%, at least 2.8%, at least 2.9%, at least 3%, at least 3.1%, at least 3.2%, at least 3.3%, at least 3.4%, at least 3.5%, at least 3.6%, at least 3.7%, at least 3.8%, at least 3.9%, at least 4%, at least 4.1%, at least 4.2%, at least 4.3%, at least 4.4%, at least 4.5%, at least 4.6%, at least 4.7%, at least 4.8%, at least 4.9%, at least 5%, at least 5.1%, at least 5.2%, at least 5.3%, at least 5.4%, at least 5.5%, at least 5.6%, at least 5.7%, at least 5.8%, at least 5.9%, at least 5.9%, at least 6.0%, at least 6.1%, at least 6.2%, at least 6.3%, at least 6.4%, at least 6.5%, at least 6.6%, at least 6.7%, at least 6.8%, at least 6.9%, at least 7.0%, at least 7.1%, at least 7.2%, at least 7.3%, at least 7.4%, at least 7.5%, at least 7.6%, at least 7.7%, at least 7.8%, at least 7.9%, at least 8.0%, at least 8.1%, at least 8.2%, at least 8.3%, at least 8.4%, at least 8.5%, at least 8.6%, at least 8.7%, at least 8.8%, at least 8.8%, at least 9.0%, at least 9.1%, at least 9.2%, at least 9.3%, at least 9.4%, at least 9.5%, at least 9.6%, at least 9.7%, at least 9.8%, at least 9.8%, at least 9.9%, or at least 10% fully human (or substantially human) immunoglobulin by mass of total immunoglobulin in the polyclonal immunoglobulin.

In some embodiments of the methods and compositions of the disclosure, the polyclonal immunoglobulin comprises 0.1-0.6%, 0.2-0.7%, 0.3-0.8%, 0.4-0.9%, 0.5-1%, 0.6-1.1%, 0.7-1.2%, 0.8-1.3%, 0.9-1.4%, 1-1.5%, 1.1-1.6%, 1.2-1.7%, 1.3-1.8%, 1.4-1.9%, 1.5-2%, 1.6-2.1%, 1.7-2.2%, 1.8-2.3%, 1.9-2.4%, 2-2.5%, 2.1-2.6%, 2.2-2.7%, 2.3-2.8%, 2.4-2.9%, 2.5-3%, 2.6-3.1%, 2.7-3.2%, 2.8-3.3%, 2.9-3.4%, 3-3.5%, 3.1-3.6%, 3.2-3.7%, 3.3-3.8%, 3.4-3.9%, 3.5-4%, 3.6-4.1%, 3.7-4.2%, 3.8-4.3%, 3.9-4.4%, 4-4.5%, 4.1-4.6%, 4.2-4.7%, 4.3-4.8%, 4.4-4.9%, 4.5-5%, 4.6-5.1%, 4.7-5.2%, 4.8-5.3%, 4.9-5.4%, 5-5.5%, 5.1-5.6%, 5.2-5.7%, 5.3-5.8%, 5.4-5.9%, 5.5-6%, 5.6-6.1%, 5.7-6.2%, 5.8-6.3%, or 5.9-6.4% fully human (or substantially human) immunoglobulin by mass of total immunoglobulin in the polyclonal immunoglobulin.

In some embodiments of the methods and compositions of the disclosure, the polyclonal immunoglobulin comprises 0-0.5%, 0.5-1%, 1-1.5%, 1.5-2%, 2-2.5%, 2.5-3%, 3-3.5%, 3.5-4%, 4-4.5% 4.5-5%, 5-5.5%, 5.5-6%, 6-6.5%, 6.5-7%, 7-7.5%, 7.5-8%, 8-8.5%, 8.5-9%, 9-9.5%, 9.5-10% or greater fully human (or substantially human) immunoglobulin by mass of total immunoglobulin in the polyclonal immunoglobulin.

In some embodiments of the methods and compositions of the disclosure, the polyclonal immunoglobulin comprises 0-1%, 1-2%, 2-3%, 3-4%, 4-5%, 5-6%, 6-7%, 7-8%, 8-9%, 9-10%, or greater fully human (or substantially human) immunoglobulin by mass of total immunoglobulin in the polyclonal immunoglobulin.

In some embodiments of the methods and compositions of the disclosure, the polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, the polyclonal immunoglobulin comprises 0-5%, 5-10%, 10-15%, 15-20%, 20-25%, 25-30%, 30-35%, 35-40%, or greater fully human (or substantially human) immunoglobulin by mass of total immunoglobulin in the polyclonal immunoglobulin.

In some embodiments of the methods and compositions of the disclosure, the polyclonal immunoglobulin comprises at least 1%, at least 2%, at least 3%, at least 4%, at least 5%, at least 6%, at least 7%, at least 8%, at least 9%, or at least 10% fully human (or substantially human) immunoglobulin by mass of total immunoglobulin in the polyclonal immunoglobulin.

In some embodiments of the methods and compositions of the disclosure, the polyclonal immunoglobulin comprises 1-4%, 2-5%, 3-6%, 4-7%, 5-8%, 6-9%, or 7-10% fully human (or substantially human) immunoglobulin by mass of total immunoglobulin in the polyclonal immunoglobulin.

In some embodiments of the methods and compositions of the disclosure, the polyclonal immunoglobulin comprises at least 5% fully human immunoglobulin by mass of total immunoglobulin in the polyclonal immunoglobulin.

In some embodiments of the methods and compositions of the disclosure, the polyclonal immunoglobulin comprises 2% to 5% fully human immunoglobulin by mass of total immunoglobulin in the polyclonal immunoglobulin.

In some embodiments, the ungulate-derived polyclonal immunoglobulin comprises “chimeric” human immunoglobulin having a human heavy chain and an ungulate kappa light chain (termed “cIgG”). In some embodiments, the polyclonal immunoglobulin comprises less than about 0.5%, less than about 0.75%, less than about 1.0%, less than about 1.25%, less than about 1.5%, less than about 1.75%, less than about 2.0%, less than about 2.25%, less than about 2.5%, less than about 2.75%, less than about 3.0%, less than about 3.25%, less than about 3.5%, less than about 3.75%, or less than about 4.0% cIgG as a percent of total protein concentration. In some embodiments, the polyclonal immunoglobulin comprises about 0.5% to about 1.0%, about 1.0% to about 1.5%, about 1.5% to about 2.0%, about 1.5% to about 2.0%, about 2.0% to about 2.5%, or about 2.5% to about 3.0% cIgG as a percent of total protein concentration. In some embodiments, the polyclonal immunoglobulin comprises about 0.5% to about 1.0%, about 1.0% to about 2.0%, or about 1.0 to about 3.0% cIgG as a percent of total protein concentration.

In some embodiments, the polyclonal immunoglobulins of the disclosure are less potent in a complement-dependent cytotoxicity (CDC) assay than a reference product (e.g. human-derived polyclonal immunoglobulin). In some embodiments, the polyclonal immunoglobulins of the disclosure are at most about 5%, at most about 10%, at most about 25%, at most about 50%, at most about 100%, at most about 150%, or more at most about 200% potent in a complement-dependent cytotoxicity (CDC) assay than a reference product (e.g. human-derived polyclonal immunoglobulin).

In some embodiments, the polyclonal immunoglobulins of the disclosure generates lower toxicity towards CD8+ cells than a reference product (e.g. human-derived polyclonal immunoglobulin. In some embodiments, the polyclonal immunoglobulins of the disclosure are at most about 5%, at most about 10%, at most about 25%, at most about 50%, at most about 100%, at most about 150%, or at most about 200% more potent in CD8+ cell killing assay than a reference product (e.g. human-derived polyclonal immunoglobulin).

In some embodiments, the polyclonal immunoglobulins of the disclosure generated lower rates of CD4+ T cell apoptosis than a reference product (e.g. human-derived polyclonal immunoglobulin. In some embodiments, the polyclonal immunoglobulins of the disclosure are at least about 5%, at least about 10%, at least about 25%, at least about 50%, at least about 100%, at least about 150%, or at least about 200% less toxic in a CD4+ cell apoptosis assay than a reference product (e.g. human-derived polyclonal immunoglobulin).

In some embodiments, the polyclonal immunoglobulins of the disclosure better preserves T_{reg} to conventional T cell rations than a reference product (e.g. human-derived polyclonal immunoglobulin. In some embodiments, the polyclonal immunoglobulins of the disclosure are at least about 5%, at least about 10%, at least about 25%, at least about 50%, at least about 100%, at least about 150%, or at least about 200% less toxic to T_{reg} cells than a reference product (e.g. human-derived polyclonal immunoglobulin).

In some embodiments of the methods and compositions of the disclosure, the population of fully human immunoglobulins (or substantially human) specifically binds human coronavirus, coronavirus S protein, or another coronavirus antigen. In some embodiments, the population of fully human (or substantially human) immunoglobulins specifically binds a human coronavirus (e.g., SARS-CoV-2), human coronavirus S protein, or another human coronavirus antigen.

In some embodiments, a genome of the transgenic ungulate comprises a human immunoglobulin locus.

In some embodiments, the transgenic ungulate is immunized 3, 4, 5, or more times.

In some embodiments, the population of fully human or substantially human immunoglobulins are purified from the serum of the transgenic ungulate after immunization.

The disclosure provides methods of providing human polyclonal immunoglobulin specific for coronavirus protein (such as coronavirus S protein) treatment to a subject in need thereof, comprising administering to the subject a polyclonal immunoglobulin according to the disclosure. In some embodiments, the method provides an effective amount of human polyclonal immunoglobulin specific for coronavirus protein to the subject.

The disclosure provides methods of providing human polyclonal immunoglobulin specific for coronavirus protein (such as coronavirus S protein) treatment to a subject in need thereof, comprising administering to the subject a composition produced by immunizing a transgenic ungulate with human coronavirus. In some embodiments, the method provides an effective amount of human polyclonal immunoglobulin specific for coronavirus protein to the subject.

The disclosure provides methods of providing human polyclonal immunoglobulin specific for coronavirus protein (such as coronavirus S protein) treatment to a subject in need thereof, comprising administering to the subject a polyclonal immunoglobulin produced according to the disclosure. In some embodiments, the method provides an effective amount of human polyclonal immunoglobulin specific for coronavirus S protein to the subject.

The disclosure further provides pharmaceutical compositions, comprising a population of fully human or substantially human immunoglobulins, and one or more pharmaceutically acceptable excipients. In some embodiments, the population of fully human or substantially human immunoglobulins specifically binds human coronavirus, human coronavirus S protein, or another human coronavirus antigen.

In some embodiments, the pharmaceutical composition comprises at least about 1 mg/mL, at least about 50 mg/mL, at least about 100 mg/mL, or at least about 1,000 mg/mL of fully human or substantially human immunoglobulin. In some embodiments, the pharmaceutical composition comprises at least about 100 μg/mL, at least about 250 μg/mL, at least about 500 μg/mL, at least about 750 μg/mL, or at least about 1,000 μg/mL of fully human or substantially human immunoglobulin.

In some embodiments, the fully human or substantially human immunoglobulin is produced in an ungulate. In some embodiments, the ungulate is a bovine.

In some embodiments, the pharmaceutical composition comprises at least 5% fully human immunoglobulin by mass of total immunoglobulin in the pharmaceutical composition.

In some embodiments, the pharmaceutical composition comprises 2% to 5% fully human immunoglobulin by mass of total immunoglobulin in the pharmaceutical composition.

### EXAMPLES

The following specific examples are to be construed as merely illustrative, and not limitative of the remainder of the disclosure in any way whatsoever.

### Example 1

### Production of Human Polyclonal Immunoglobulin in Transchromosomic Bovine (TcB) System

Applicant has developed a transchromosomic (Tc) bovine production system in which bovine Ig genes are knocked-out and a human artificial chromosome (HAC) vector is introduced into the bovine genome to express human polyclonal antibodies. These Tc cattle are immunized with specific targets, such as an ectodomain of the spike (S) protein of SARS-CoV-2, to produce substantial amounts of antigen specific-human polyclonal antibodies for therapeutic treatments.

Transchromosomic (Tc) bovines (qualified as producing >2 mg/mL of total human IgG, >30% IgG1) were immunized initially (vaccinations 1 and 2) with a plasmid DNA (pDNA) vaccine that expresses wild-type SARS-CoV-2 spike protein, followed by additional immunizations (vaccinations 3 and beyond) with a recombinant spike protein from SARS-CoV-2 produced in insect cells, according the schedule in Table 1.

TABLE 1
Vaccination Interval and Formulation

| Antigen | | |
|---|---|---|
| Type | Vaccination | Vaccine Formulation |
| pDNA | V1 to V2 | 12 mg SARS-CoV-2 pDNA + |
| | with 3-4 week | ISA-206 50%/Quil A 2 mg |
| | interval | |
| Spike | V3 to V5 | 2 to 5 mg SARS-CoV-2 |
| protein | with 4 week | recombinant spike protein + |
| | interval | ISA-206 50%/Quil A 2 mg |

For the first and second vaccinations (V1 and V2), pDNA encoding full-length spike (S) protein from Severe Acute Respiratory Syndrome-related Coronavirus 2 (SARS-CoV-2), Wuhan-Hu-1 (GenPept: QHD43416), was administered by intramuscular (IM) injection. The adjuvants Montanide ISA-206 and Quil A were co-administered adjacent to the DNA vaccination site.

For the third vaccination (V3), a recombinant S protein from SARS-CoV-2, Wuhan-Hu-1 (GenPept: QHD43416) mixed with Montanide ISA-206 and Quil A was administered. The S protein was produced in insect cells using a baculovirus expression system and purified by nickel affinity chromatography. This recombinant protein contains 1196 residues (ectodomain) of the SARS-CoV-2 S protein. It was modified to remove the polybasic S1/S2 cleavage site (RRAR [SEQ ID NO:18] to A; residues 682 to 685), stabilized with a pair of mutations (K986P and V987P, wild type numbering) and includes a thrombin cleavage site, T4 foldon trimerization domain and C-terminal hexa-histidine tag.

Collected plasma was frozen for shipment, thawed, pooled, fractionated by caprylic acid (CA), and clarified by depth filtration. Clarified material containing Tc bovine-derived human immunoglobulin G (IgG) was purified by affinity chromatography, first using an anti-human IgG affinity column to bind Tc bovine-derived human IgG (hIgG) and remove bovine plasma proteins (BPP) followed by a low pH treatment for viral inactivation, and, then, by passing through an anti-bovine IgG (bIgG) heavy chain (HC) specific affinity column to further remove residual IgG molecules that contain a bovine HC or Fc of bovine HC. The Tc bovine-derived human IgG fraction was then concentrated and diafiltered prior to a Q Sepharose chromatography polishing step, nanofiltration, final buffer exchange, concentration and sterile filtration.

### Biochemical Characterization of TcB-Produced Human Polyclonal Immunoglobulin

The Tc bovine was engineered to be biased to human IgG1. Human IgG subclasses ratios for normal human plasma and Tc bovine are presented in Table 2. In Tc bovine derived antibodies, the IgG subclasses are in the following ranges:

TABLE 2
IgG Subclasses Present in Normal Human
IgG Compared to Tc Bovine Human IgG

| IgG | Normal | Human IgG from |
|---|---|---|
| Species | Human IgG % | Tc Bovine % |
| IgG1 | 60.3-71.5 | 70.3-98.6 |
| IgG2 | 19.4-31.0 | 1.4-34.5 |
| IgG3 | 5.0-8.4 | 0.0-0.3 |
| IgG4 | 0.7-4.2 | 0.0-0.4 |

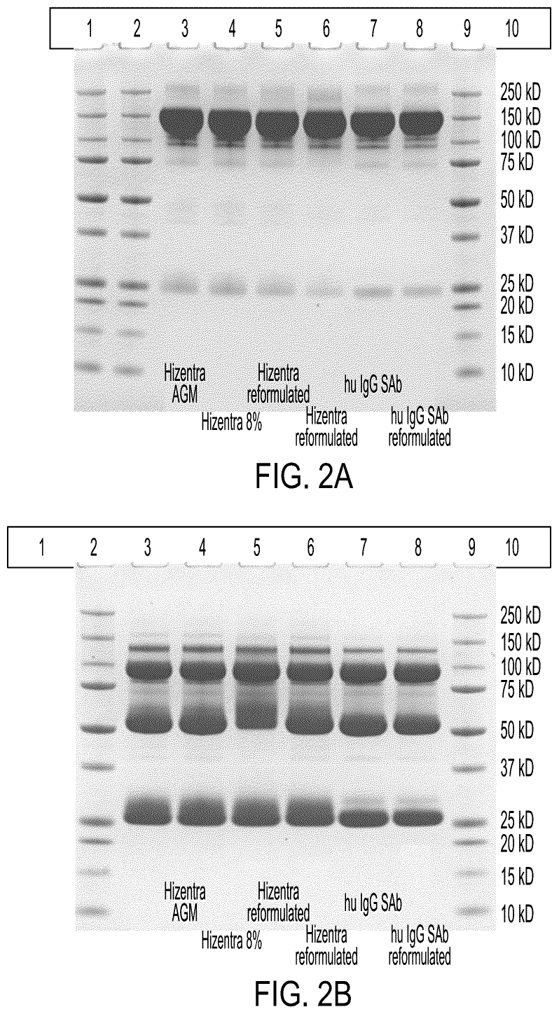

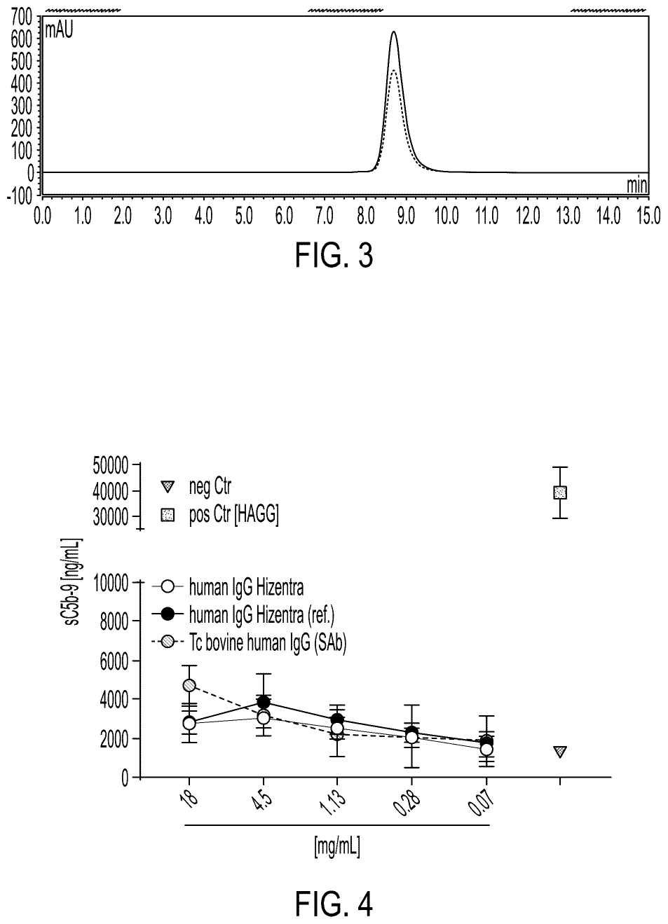

Purified immunoglobulin was characterized by SDS-PAGE (FIGS. 2A-2B) and size exclusion chromatograph (FIG. 3). Human immunoglobulin is present at high concentration (Table 3) and high monomer abundance and IgG1 percentage (Table 4).

TABLE 3

| Analytical | | Hizentra | Hizentra re- | Tc bovine hlgG |
|---|---|---|---|---|
| parameter | Unit | original | formulated | re-formulated |
| Protein conc., | mg/mL | 204.2 | 201.7 | 192.8 |
| A280 | | | | |
| Protein conc., | mg/mL | 1933 | 208.8 | 188.5 |
| Biuret | | | | |
| Volume | mL | n.a. | 23 | 35 |
| L-proline | mmol/L | 254 | 219 | 218 |
| P80 | mg/L | 15 | 26 | 67 |
| pH | (1% protein | 4.85 | 5.59 | 6.09 |
| | in NaCl | | | |
| | 0.9%) | | | |
| Viscosity | mPa*s | 15 | 21 | 14 |
| Endotox LA L | IU/mL | <0.2 | <0.05 | <0.5 |
| Endotox | IU/mg | <0.001 | <0.005 | <0.005 |
| Endosafe | protein | | | |

TABLE 4

| | | | Hizentra | Tc bovine |
|---|---|---|---|---|
| Analytical | | Hizentra | re- | hlgG re- |
| parameter | Unit | original | formulated | formulated |
| SEC-HPLC | % Aggregates | <0.1 | <0.1 | <0.1 |
| % | % Dimers | 8.98 | 13.14 | 0.76 |
| | % Monomers | 91.02 | 86.86 | 99.24 |
| | % Fragments | <0.1 | <0.1 | <0.1 |
| lgG subclasses | % lgG1 | | 70.6 | 93.5 |
| | % lgG2 | | 26.3 | 6.5 |
| | % lgG3 | | 1.6 | 0 |
| | % lgG4 | | 1.6 | 0 |
| Complement activ. | % ACA | 14 | 15 | 16 |
| HBs-Ab | IU/mL | 14.6 | 9.2 | not detectable |
| Parvovirus-Ab | IU/mL | 595 | 509 | 5 |
| Diphteria a antitoxin | IU/mL | 18.1 | 18.1 | 57.8 |

### Functional Characterization of TcB-Produced Human Polyclonal Immunoglobulin

Binding to human Fe gamma receptors was assay by biolayer interferometry. Results are shown in Table 5.

TABLE 5

| | | | Kₒₙ | K_{dis} | Calculation |
|---|---|---|---|---|---|
| Receptor | K_{D} | | (1/Ms) | (1/s) | method |
| lgG (Hizentra) | | | | | |
| FcγRI (CD64) | 19.7 nM ± | 2.0 | 8.7 × 10⁴ | 1.7 × 10⁻³ | Kinetic fit |
| FcγRIIa | 212.7 nM ± | 7.4 | 2.6 × 10⁵ | 5.5 × 10⁻² | Kinetic fit |
| (CD32a) | | | | | |
| FcγRIIb/c | 2.7 μM ± | 0.3 | n/a | n/a | Steady state |
| (CD32b/c) | | | | | |
| FcγRIIIa | 495.9 nM ± | 42.6 | 7.3 × 10⁴ | 3.6 × 10⁻² | Kinetic fit |
| (CD16a) | | | | | |
| FcRn | 1.0 nM ± | 0.06 | 2.5 × 10⁶ | 2.5 × 10⁻³ | Kinetic fit |
| lgG (SAb) | | | | | |
| FcγRI (CD64) | 19.1 nM ± | 3.2 | 1.0 × 10⁵ | 1.9 × 10⁻³ | Kinetic fit |
| FcγRIIa | 535.0 nM ± | 281.7 | n/a | n/a | Steady state |
| (CD32a) | | | | | |
| FcγRIIb/c | 5.3 μM ± | 0.6 | n/a | n/a | Steady state |
| (CD32b/c) | | | | | |
| FcγRIIIa | 1.3 μM ± | 0.09 | 1.6 × 10⁵ | 2.0 × 10⁻¹ | Kinetic fit |
| (CD16a) | | | | | |
| FcRn | 1.7 nM ± | 0.1 | 2.0 × 10⁶ | 3.4 × 10⁻³ | Kinetic fit |

Complement activation was assessed by detecting sC5b-9 by ELISA in whole blood. Blood was incubated in the present of polyclonal immunoglobulin for two hours at 37° C., 25 mM. EDTA is added to stop the reaction (Complement activation is Ca2+ and Mg2+ dependent). Heat aggregated IgG (HAGG) was used as positive control for complement activation. Activation is measured by analyzing complement fragments as sC5b-9 by ELISA. No statistical difference was observed between human-derived and TcB samples (FIG. 4).

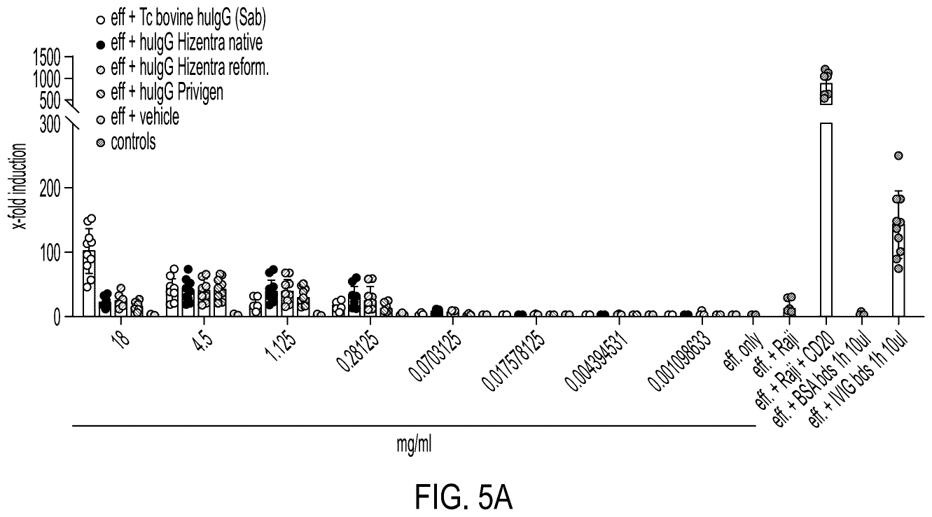

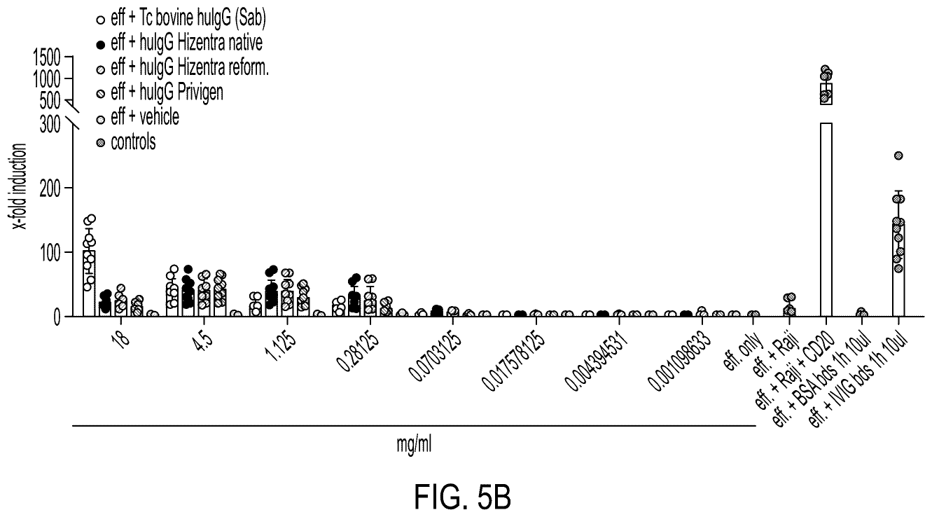

Antibody-dependent cellular cytotoxicity (ADDC) was assayed by detecting CD16A-mediated signaling with an ADCC iLite assay (FIG. 5A) and CD32A-mediated signaling using an assay from Promega® (FIG. 5B). No statistical difference was observed between human-derived and TcB samples.

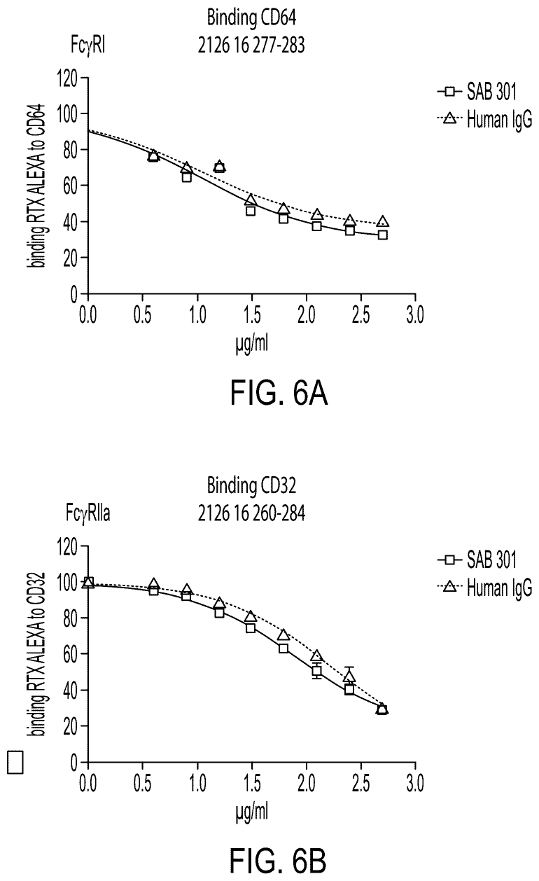

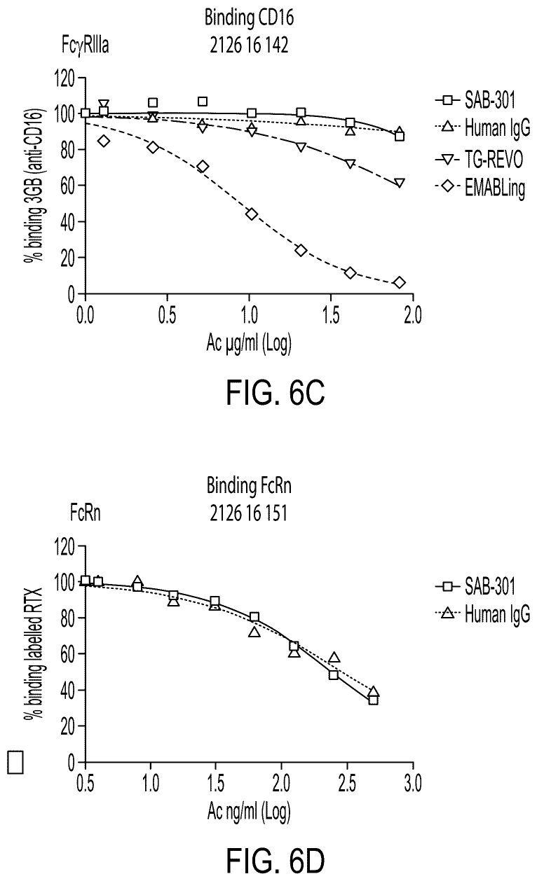

Further data demonstrating similar levels of human Fc gamma receptor binding is provided in FIGS. 6A-6B.

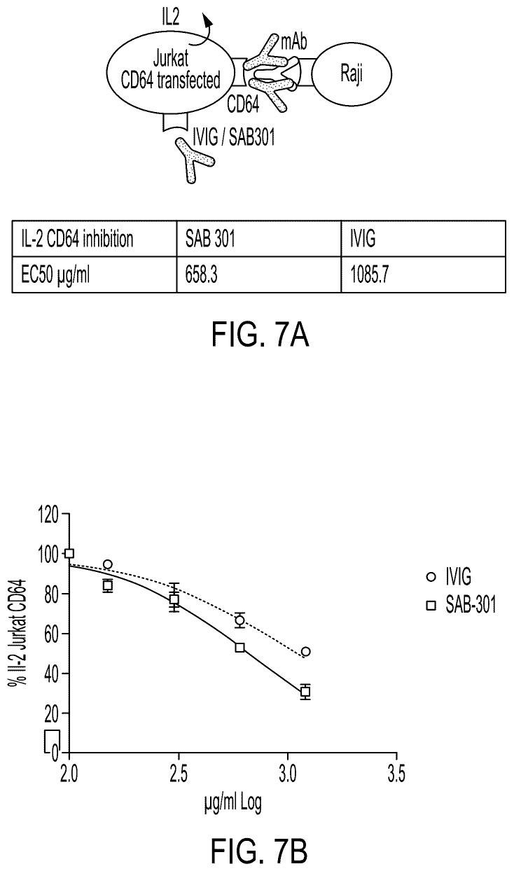

Inhibition of IL-2 production produced by Jurkat-CD64 in the presence of Raji and anti-CD20 Rituxan was also tested. TcB-derived product demonstrated slightly better inhibition of immune complex interaction with effector cells specifically inhibition of IL-2 production compared to human IgG (FIG. 7).

### Glycosylation of TcB-Produced Human Polyclonal Immunoglobulin

Reduction of fucosylation leads of IgG1 have been shown to increased macrophage activation and lung epithelial damage in COVID-19 patients.

Applicants have discovered that, unlike convalescent serum, the ungulate-derived polyclonal immunoglobulin described here, while having a fully human antibody sequence, maintains higher fucosylation. The TcB system maintains high fucosylation at 94% comparable to normal human IgG1 fucosylation level, but distinct from the fucosylation levels observed in human-derived hIgG1 specific to SARS-CoV-2. The disclosure therefore provides a unique method to obtain highly fucosylated human immunoglobulin for treatment of COVID, such as COVID-19 or disease cause by future coronavirus strains. Normal levels of IgG1 fucosylation (e.g., ˜94%) distinguish this therapy from others as they may avoid strong activation of ADCC and effector cell function that could lead to epithelial lung tissue damage.

Additionally, TcB-derived produce contains NGNA, which distinguishes the product from human-derived products.

Table 6 provides the glycosylation profile of the TcB-derived product determined by mass spectrometry.

TABLE 6

| Glycosylational features | % |
|---|---|
| Sialylated structures | 14.0 |
| α-Gal structures | 2.3 |
| N-glycolyneuraminic (NG)-cont. Structures | 13.8 |
| N-acetylneuraminic (NA)-cont. Structures | 0.2 |
| Core-fucosylated structures | 94.4 |
| Bisected structures | 14.5 |
| Highmannose structures | 2.9 |
| Hybrid structures | 1.4 |
| Complex structures | 95.7 |

Table 7 compares the NGNA content of the TcB-derived product to human-derived product. Sialic Acid analysis indicates TcB-produced hIgG has >90% NGNA. The only impact expected of NGNA would be higher clearance but prior clinical work demonstrates that TcB-derived product has a half-life of 28 days, the same as human-derived hIgG1.

TABLE 7

| | NGNA | NANA |
|---|---|---|
| Sample name | contents | content |
| human IgG from #468 | 0.90 | 0.04 |
| Chimeric IgG from #468 | 0.71 | 0.03 |
| Bovine IgG | 0.64 | 0.12 |
| Human IVIG | 0.00 | 0.72 |
| KRN330 (mAb)* | 0.03 | 0.13 |
| NESP* | 0.2 | 20 |

### Example 2

### Safety, Tolerability, and Pharmacokinetics of SAB-185 in Healthy Participants

Coronavirus disease 2019 (COVID-19) is an infectious disease caused by severe acute respiratory syndrome coronavirus 2 (SARS-CoV-2). SAB Biotherapeutics has developed SAB-185, an Anti-SARS-CoV-2 Human Immunoglobulin Intravenous (transchromosomic [Tc] bovine-derived), as a potential therapeutic to treat COVID-19. This study will evaluate the safety, immunogenicity, and pharmacokinetics of SAB-185 in healthy participants.

Five cohorts received escalating dose levels or product, or placebo:
10 mg/kg SAB-185 in normal (0.9%) saline; concentration 4 mg/mL (0.4%)
25 mg/kg SAB-185 in normal (0.9%) saline; concentration 20 mg/mL (2%)
25 mg/kg SAB-185 in normal (0.9%) saline; concentration 20 mg/mL (2%). Cohort 3 will receive a second 25 mg/kg dose of SAB-185 7 days (+/−2) after the first treatment.
50 mg/kg SAB-185 in normal (0.9%) saline; concentration 20 mg/mL (2%)
Normal (0.9%) saline in approximately the same volume as each cohort in the experimental drug arm.

Primary outcome measures are:
Number of Participants Having Adverse Events [Time Frame: 29 Days]
Incidence and severity of other adverse events and severe adverse events (SAE)
Number of Participants Having Transfusion-Related Adverse Events [Time Frame: 29 Days]
Transfusion-related adverse events

Secondary outcome measures are:
Number of Participants Having Adverse Events [Time Frame: 90 Days]
Incidence and severity of adverse events and SAEs from Screening through Study Day 90
Pharmacokinetics from screening to day 90 [Time Frame: 90 Days]
SARS-CoV-2 binding (ELISA) and neutralizing (PRNT80) antibody titers from Screening through Study Day 90

### Safety, Tolerability, and Pharmacokinetics of SAB-185 in Ambulatory Participants With COVID-19

Coronavirus disease 2019 (COVID-19) is an infectious disease caused by severe acute respiratory syndrome coronavirus 2 (SARS-CoV-2). SAB Biotherapeutics has developed SAB-185, an Anti-SARS-CoV-2 Human Immunoglobulin Intravenous (transchromosomic [Tc] bovine-derived), as a potential therapeutic to treat COVID-19. This study will evaluate the safety, immunogenicity, and pharmacokinetics of SAB-185 in ambulatory participants with COVID-19.

SAB-185 is a purified human immunoglobulin G (hIgG) designed to specifically bind to Severe Acute Respiratory Syndrome Coronavirus 2 (SARS-CoV-2) viruses. SAB-185 is purified from the plasma of immunized Tc bovines that were immunized initially (vaccinations 1 and 2) with a plasmid DNA (pDNA) vaccine that expresses wild-type SARS-CoV-2 spike protein, followed by additional immunizations (vaccinations 3 and beyond) with a recombinant spike protein from SARS-CoV-2 produced in insect cells. The purified hIgG is a sterile liquid formulated in 10 mM glutamic acid monosodium salt, 262 mM D-sorbitol, 0.05 mg/mL Tween 80, pH 5.5. The drug product will be administered intravenously and will be diluted in saline per the clinical protocol.

Four cohorts received escalating dose levels or product, or placebo:
10 mg/kg SAB-185 in normal (0.9%) saline; concentration 4 mg/mL (0.4%)
25 mg/kg SAB-185 in normal (0.9%) saline; concentration 20 mg/mL (2%)
50 mg/kg SAB-185 in normal (0.9%) saline; concentration 20 mg/mL (2%)
Normal (0.9%) saline in approximately the same volume as each cohort in the experimental drug arm.

Primary Outcome Measures are:
Number of Participants Having Adverse Events [Time Frame: 29 Days]
Incidence and severity of other adverse events and severe adverse events (SAE)
Number of Participants Having Transfusion-Related Adverse Events [Time Frame: 29 Days]
Transfusion-related adverse events

Secondary outcome measures are:
Number of Participants Having Adverse Events [Time Frame: 90 Days]
Incidence and severity of adverse events and SAEs from Screening through Study Day 90 Assessment of the PD of SAB-185 administered intravenously [Time Frame: 90 Days]
Measurement of SARS CoV-2 neutralizing (PRNT80) antibody titers from screening through Study Day 90
Immune response elicited by SAB-185 [Time Frame: 90 Days]
Measurement of Rheumatoid factor through day 90
Concentration of subject anti-SAB-185 antibodies elicited by SAB-185 [Time Frame: 90 Days]
Measurement of anti-SAB-185 antibodies through screening day 90
Incidence of SARS-CoV-2 in oropharyngeal (OP) or nasopharyngeal (NP) swab specimens [Time Frame: 29 Days]
Incidence of SARS-CoV-2 in swab specimens as measured by quantitative RT-PCR through Study Day 29
Level of SARS-CoV-2 in oropharyngeal (OP) or nasopharyngeal (NP) swab specimens [Time Frame: 29 Days]
Level of SARS-CoV-2 in swab specimens as measured by quantitative RT-PCR through Study Day 29

### Example 3

### SAB-185 In Vitro Evaluation Against VSV-SARS-CoV-2 Mutants

Four mutants of concern including (D614G, N501Y, S477N and E484K) are currently highly circulating in humans In this study, we used VSV-SARS-CoV-2 and SAB-185, which was purified from transchromosomic (Tc) bovines hyperimmunized with two doses of plasmid DNA encoding the Wuhan strain S gene and followed by repeated doses of recombinant S protein, to evaluate the ability of SAB-185 to neutralize the wild-type SARS-CoV-2 (D614G variant) and chimeric vesicular stomatitis virus (VSV) SARS-CoV-2 reporter viruses in vitro with Wild-type and D614G, S477N, E484K, and N501Y S protein substitutions in comparison to a neutralizing receptor-binding-domain (RBD) monoclonal to the SARS-CoV-2 Wuhan-Hu-1 S protein.

The genome of transchromosomal (Tc) bovines contains a human artificial chromosome (HAC) comprising the entire human Ig gene repertoire (human Ig heavy chain [IgH] and human kappa light chain) that reside on 2 different human chromosomes (hChr), specifically the IgH locus from hChr14 and the Immunoglobulin kappa (Igk) locus from hChr2. The system maintains the ability to use the genetic information provided by the immunoglobulin gene repertoires for generating a wide diversity of human polyclonal antibodies (pAbs). Fully hIgG (hIgG/hIgx) can then be produced in these Tc bovines after vaccination with suitable antigens, and these animals produce up to 15 g/L of IgG antibodies in their plasma (similar to humans which have 7-16 g/L IgG). Using this approach polyclonal sera against a swath of emergent threats to humans have been developed and established as safe.

### Results

### Generation of Human Immunoglobulin Against SARS-CoV-2 Spike in Transchromosomic Bovines.

To generate anti-SARS-CoV-2 polyclonal human immunoglobulin SAB-185, we primed transchromosomic (Tc) bovines with a DNA encoding the Wuhan-Hu-1 strain Spike (S) gene (Wu et al. Nature 579:265-69 (2020)). SARS-CoV-2 spike protein for the first vaccination (V1) and the second vaccination (V2) at a 3-week interval, followed by 3 subsequent boosts with recombinant spike ectodomain produced and purified from insect cells for the third vaccination (V3) to the fifth vaccination (V5) at a 4-week interval. Plasma was collected on 8, 11 and 14 days post each booster from V3 to V5. Then the qualified plasma was pooled and subjected to cGMP purification for human IgG SAB-185. SAB-185 Lot 1 and Lot 5 were purified from pooled V3 plasma and V4 plasma, respectively. SAB-185 Lot 6 was purified from pooled V3, V4 and V5 plasma.

### Effect of SAB-185 on Neutralization of VSV-SARS-CoV-2.

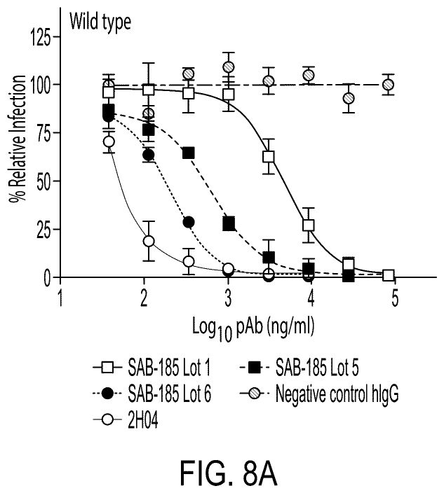

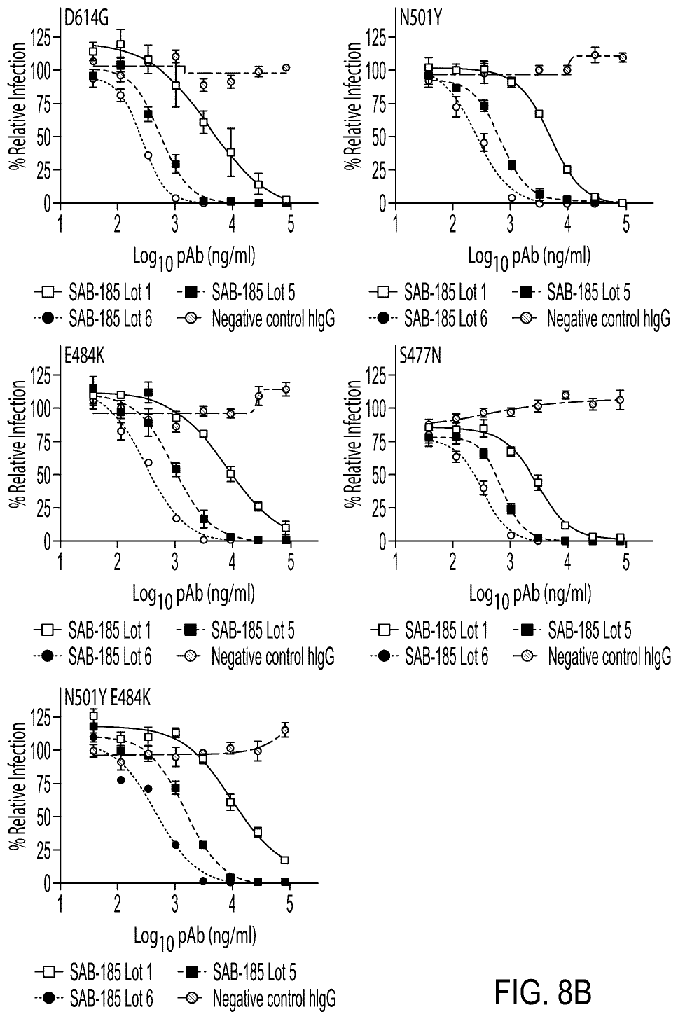

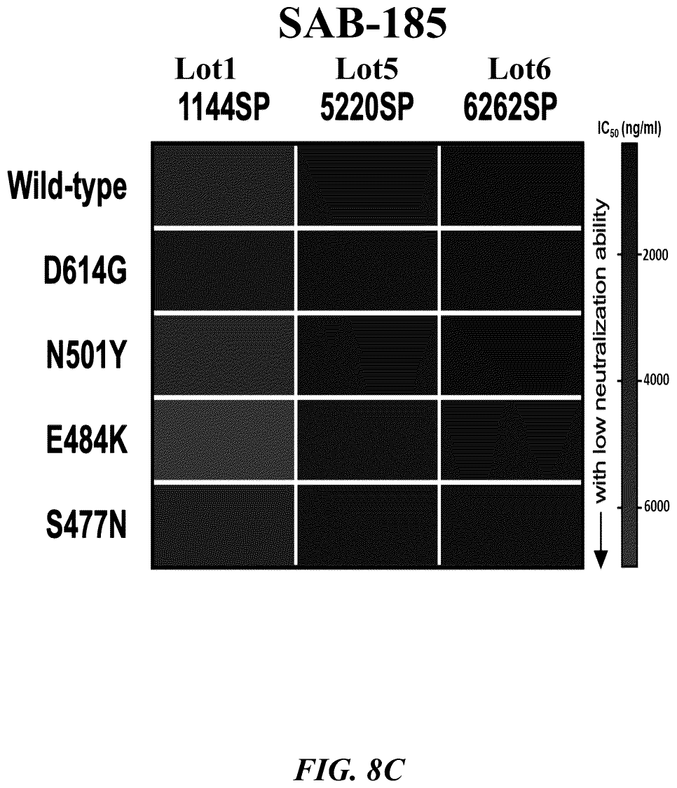

Here using a neutralization assay based on VSV-SARS-CoV-2, we evaluated the ability of SAB-185 to inhibit infection mediated by the S protein. We incubated VSV-SARS-CoV-2 with increasing concentrations of SAB-185 for 1 h at 37° C. and measured residual infectivity on Vero E6 cells. We observed effective neutralization, with an approximate IC₅₀ of 4728 ng/ml for SAR-185 Lot 1, 649.1 ng/ml for SAR-185 Lot 5 and 211.9 ng/ml for SAB-185 Lot 6, which compares to neutralization by potent monoclonal antibodies such as 2H04. (FIG. 8A).

Using monoclonal antibodies, we previously selected >50 mutants in SARS-CoV-2 spike that exhibit resistance to specific monoclonal antibodies. Among those mutants were S477N and E484K which exhibit resistance to multiple antibodies and are present in emerging variants of concern. We also generated the dominant D614G, and the mouse adapted N501Y variants. To determine whether the potency of SAB-185 was altered by any of these individual amino acid substitutions in spike, or the double mutant N501Y E484K, we performed neutralization assays. We incubated each of the indicated VSV-SARS-CoV-2 mutants with increasing concentrations of SAB-185 for 1 h at 37° C. and measured residual infectivity on Vero E6 cells (FIG. 8B). FIG. 8C is a heat map of the same data. All four mutants were dose-dependently inhibited by SAB-185, at levels that were similar to those seen for wild type spike. This data demonstrates that SAB-185 had equivalent neutralizing ability to D614G, N501Y, S477N and E484K mutants that are present in circulating human isolates of SARS-CoV-2.

### Selection of SAB-185 Escape Mutants

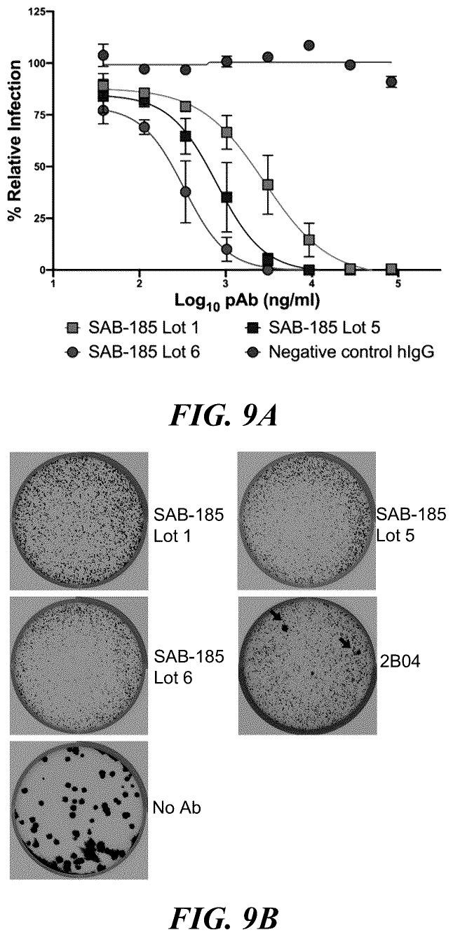

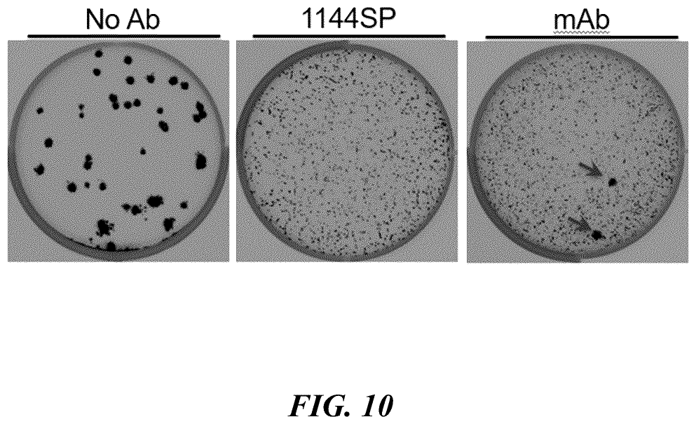

Taking advantage of the intrinsically error prone nature of the VSV RNA dependent RNA polymerase we and others have previously isolated VSV-SARS-CoV-2 S gene mutants by selection using mAbs and human convalescent serum. SAB-185 and a negative control hIgG were tested for neutralizing activity against VSV-SARS-CoV-2 at the MOI of 1. The concentration of SAB-185 added in the overlay completely inhibited viral infection (FIG. 9A). In contrast to the ability to readily isolate mAb and serum escape mutants, we were unable to isolate mutants resistant to the human immunoglobulin SAB-185 (FIG. 9B). Taken together, this analysis suggests that natural cocktails of pAbs binding distinct epitopes on SARS-CoV-2 S protein pose a barrier to immune escape.

### Experimental Model and Subject Details

Cells. Cells were cultured in humidified incubators at 340 or 37° C. and 5% CO₂ in the indicated media. Vero CCL81, Vero E6 and Vero E6-TMPRSS2 were maintained in DMEM (Corning or VWR) supplemented with glucose, L-glutamine, sodium pyruvate, and 10% fetal bovine serum (FBS). MA104 cells were propagated in Medium 199 (Gibco) containing 10% FBS. Vero E6-TMPRSS2 cells were generated using a lentivirus vector described as previously (6).

VSV-SARS-CoV-2 mutants. VSV-SARS-CoV-2 was described as previously (6). S477N and E484K were escape mutants isolated from mAbs described as previously (7). N501Y and D614G were constructed using SARS-CoV-2 Wuhan-Hu-1 spike with substitution at N501 or D614 site respectively and rescued by using reverse genetic system. Virus were then plaque purified and the mutations were identified by Sanger sequencing (GENEWIZ). Viral stocks were amplified on MA104 cells at an MOI of 0.01 in Medium 199 containing 2% FBS and 20 mM HEPES pH 7.7 (Millipore Sigma) at 34° C. Viral supernatants were harvested upon extensive cytopathic effect and clarified of cell debris by centrifugation at 1,000×g for 5 min. Aliquots were maintained at −80° C.

### Method Details

Plaque assays. Plaque assays were performed on Vero and Vero E6-TMPRSS2 cells. Briefly, cells were seeded into 6 well plates for overnight. Virus was serially diluted using DMEM and cells were infected at 37° C. for 1 h. Cells were cultured with an agarose overlay in the presence of Ab or absence of Ab at 34° C. for 2 days. The concentration of SAB-185 pAbs added in the overlay completely inhibited viral infection. Plates were scanned on a biomolecular imager and expression of eGFP is show at 48 hours post-infection.

Neutralization assays using a recombinant VSV-SARS-CoV-2 and mutants. Briefly, the initial dilution of started at 83 μg/mL and was three-fold serially diluted in 96-well plates over eight dilutions. Indicated dilutions of SAB-185 pAbs were incubated with 10² PFU of VSV-SARS-CoV-2 and mutants for 1 h at 37° C. SAB-185 pAb-virus complexes then were added to Vero E6 cells in 96-well plates and incubated at 37° C. for 7.5 h. Cells were fixed at room temperature in 2% formaldehyde containing 10 μg/mL of Hoechst 33342 nuclear stain for 45 min. Fixative was replaced with PBS prior to imaging. Images were acquired using an In Cell 2000 Analyzer automated microscope (GE Healthcare) in both the DAPI and FITC channels to visualize nuclei and infected cells (×4 objective, 4 fields per well). Images were analyzed using the Multi Target Analysis Module of the In Cell Analyzer 1000 Workstation Software (GE Healthcare). GFP-positive cells were identified using the top hat segmentation method and counted within the InCell Workstation software.

### Quantification and Statistical Analysis

All statistical tests were performed as described in the indicated figure legends. Non-linear regression (curve fit) was performed for FIGS. 8A-8B and FIG. 9A using Prism 9.0. The number of independent experiments used are indicated in the relevant Figure legends.

### Example 4

Intramuscular Anti-SARS-CoV-2 Immunoglobulin from Transchromosomic Bovines Protects K18 Transgenic Mice Against Live Virus Challenge

An anti-SARS-COV-2 product (SAB-185) was produced. SAB-185 was evaluated in a Phase 1 healthy volunteer clinical trial and a Phase 1b ambulatory COVID-19 patient clinical trial using the IV route of administration up to 50 mg/kg [clinicaltrial.gov; NCT04469179, NCT04468958 respectively]. SAB-185 was safe, well-tolerated and non-immunogenic in healthy and COVID-19-infected volunteers (manuscript in preparation). Next a SARS-CoV-2 live virus challenge study in K18 transgenic mice expressing human ACE2 receptors was performed.

To evaluate pre-exposure effectiveness, two groups of K18 mice (n=12 per group) were given IM injections of either a low or high dose of SAB-185 and then challenged 12 hours later with a sub-lethal dose of SARS-COV-2 administered intranasally. To evaluate post-exposure effectiveness, two groups of K18 mice (n=12 per group) were treated with IM administered SAB-185 at either six or 24-hours after sub-lethal virus intranasal challenge. A fifth group of infected saline treated K18 mice served as control (n=12). IM administration of SAB-185 was highly effective in preventing SARS-COV-2 infection. A separate experiment evaluated the effectiveness of SAB-185 against lethal challenge doses of SARS-COV-2. Two groups of K18 mice (n=5) were treated with IM SAB-185 either 12 hours prior to or 6 hours after intranasal SARS-COV-2 challenge. A group of infected saline treated K18 mice (n=5) served as control. All treated mice survived compared to 1 of 5 untreated controls. These results show that SAB-185 is effective as pre- and post-exposure prophylaxis in K18 mice when administered by IM injection. These results support further clinical testing in humans to prevent COVID-19 disease.

### Results

Efficacy of SAB-185 in Reducing SARS-CoV-2 Viral Load after Challenge

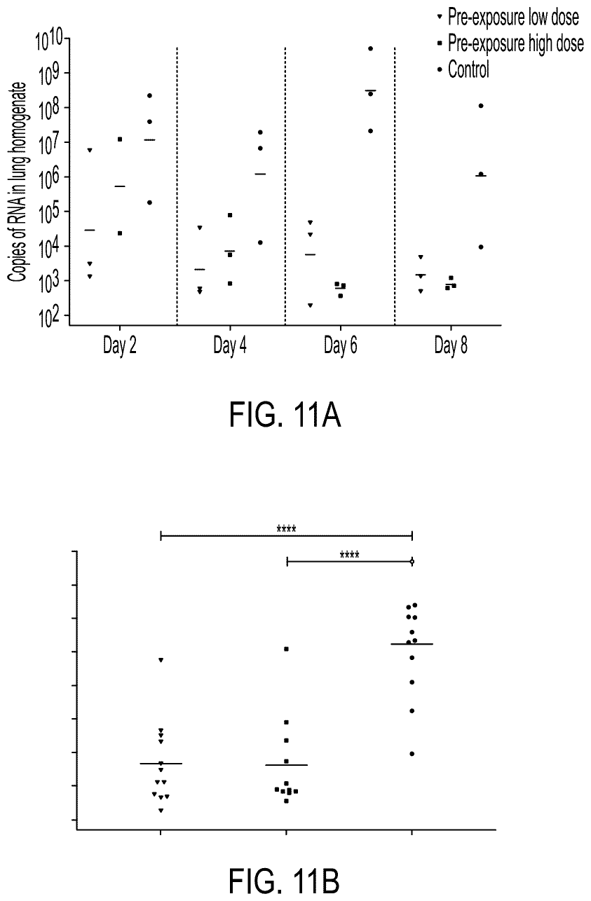

Five groups (n=12 each) of K18 transgenic mice were treated with low (1.87 mg) and high (3.75 mg) doses of SAB-185 before (pre-exposure) or with high dose SAB-185 six and 24 hours after (post-exposure) intranasal challenge with live SARS-CoV-2 virus. Within each group, three mice were euthanized at days 2, 4, 6 and 8 post-challenge. At the time of euthanasia, blood samples were obtained, and lungs, nasal turbinate, brain, liver, kidney and heart removed for analysis. Lung tissue was evaluated for the presence of SARS-Cov-2 by ddRT-PCR and virus isolation to assess the systemic antiviral effects of SAB-185 post-IM administration. FIG. 11A shows the copy number of SARS-CoV-2 RNA present in lung tissue at the various time points from animals in pre-exposure groups. At all time points, groups treated with low or high dose of SB-185 showed lower geometric mean viral RNA copy numbers compared to control saline treated group. Because there were only 3 animals at each time point, a statistical analysis was not done. FIG. 11B shows the ddRT-PCR results for all animals (n=12) in the high and low dose pre-exposure groups compared to the control animals. The geometric mean RNA copy numbers for both pre-exposure groups were similar and significantly lower (p<0.0001) than the geometric mean RNA copy number for the control saline treated mice, demonstrating the effectiveness of pre-exposure IM SAB-185 treatment.

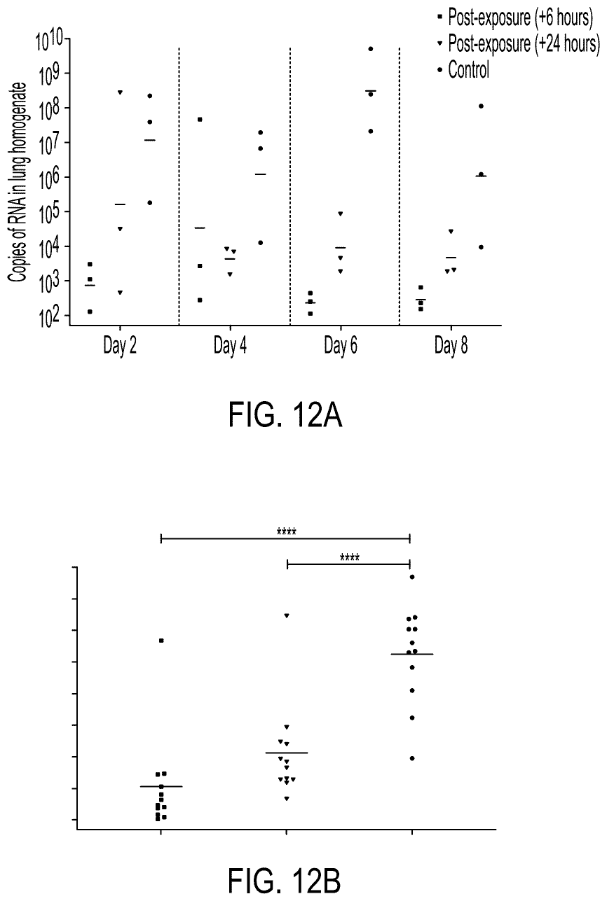

Similarly, the lungs from animals in post-exposure groups (FIG. 12A), which received high dose SAB-185 six and 24 hours after challenge, respectively, showed lower geometric mean SARS-CoV-2 RNA copies at all time points compared to control animals that received saline. FIG. 12B shows the ddRT-PCR results for all animals in each group compared to the control group, irrespective of the time sampled post challenge. The results show that the geometric mean number of SARS-CoV-2 RNA copies was significantly lower than the geometric mean copy number of the saline treated control group (p<0.0001), demonstrating the effectiveness of post-exposure IM SAB-185 treatment.

To evaluate the groups of animals for the presence of live virus in the lungs post-exposure, lung tissue was processed and cultured in VERO81 cells, followed by IFA to detect the presence of SARS-CoV-2. Results are summarized in Table 8. Lung samples from all animals in the low and high dose pre-exposure groups showed no detectable live virus through day 8 post challenge. Similarly, all lung tissues from the 6 hour post-exposure group treated with high dose IM SAB 185 were negative for detectable live virus. Lung samples from four of 12 animals in the 24 hour post exposure SAB-185 treated group had detectable live virus. Of these, two were from specimens taken on day 2 post-challenge and two were from specimens obtained on day 6. Unlike the SAB-185 treated animals, lung tissue from 9 of 12 animals in the control saline treated group had detectable live virus across all time points.

TABLE 8
Lung tissue virus isolation results from treated
and untreated mice (# positive/#total).

| Group | Day 2 | Day 4 | Day 6 | Day 8 | Total |
|---|---|---|---|---|---|
| Saline control | 2/3 | 2/3 | 3/3 | 2/3 | 9/12 |
| low dose pre-exposure | 0/3 | 0/3 | 0/3 | 0/3 | 0/12 |
| high dose pre-exposure | 0/3 | 0/3 | 0/3 | 0/3 | 0/12 |
| 6 hour post-exposure | 0/3 | 0/3 | 0/3 | 0/3 | 0/12 |
| 24 hour post-exposure | 2/3 | 0/3 | 2/3 | 0/3 | 4/12 |

Efficacy of SAB-185 on Reducing Organ Pathology after Challenge

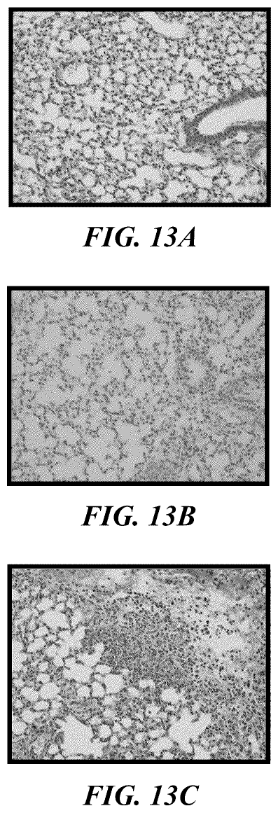

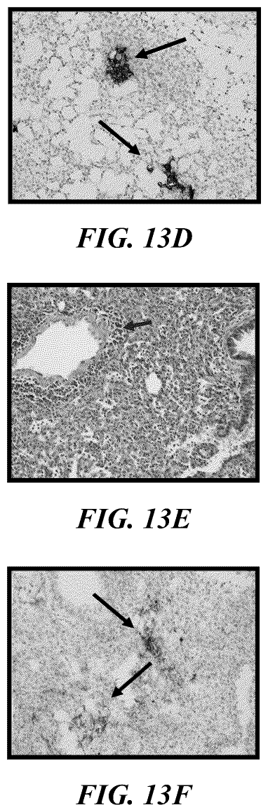

Three mice from each group were euthanized on days 2, 4, 6 and 8 post virus challenge. Nasal turbinate, lung, liver, kidney, heart, and brain tissues were examined by histologic/immuno-histologic staining for organ pathology and scored as described in Table 3. All control saline treated mice except two (one at day 2 and one at day 4) showed 1+ to 3+ histological changes in the lungs up to day 8 post-challenge. Lung tissues showed multifocal perivascular and interstitial mononuclear, eosinophilic infiltrates. Blood vessels demonstrated transmural multifocal mononuclear cells consistent with margination and transmigration of mononuclear leukocytes (FIG. 13E). Alveolar inflammation was also observed showing lymphocytic, histiocytic multifocal infiltrates consistent with interstitial pneumonia. Immunohistochemical staining for the presence of SARS-CoV-2 viral antigen showed multifocal immunoreactivity in the alveolar septa (FIG. 13F). In contrast, lung tissue from SAB-185 treated animals (Pre-Exp low and high dose, Post-Exp 6H and 24H) appeared normal except for one mouse each in the Pre-Exp high dose (day 2), Post-Exp 6H (day 4 and 8) and Post-Exp 24H (day 2 and 6) groups.

When nasal turbinate of animals was examined, all saline treated control mice showed 3+ pathology on day 2 which decreased to 1+ on day 4 and day 6. By day 8, only ⅓ animals showed abnormal histology. In general, the pathology in the nasal turbinates of SAB-185 treated animals were less severe compared to saline treated animals. Pre-exposure low dose group showed a 2+ to 3 pathology on day 2, 1+ on day 4 followed by normal nasal turbinate on days 6 and 8. In the pre-exposure high dose group, mice showed 1+ to 2+ pathology only on day 2. Mice euthanized on days 4, 6 and 8 had normal nasal turbinate. Among the post-exposure treatment groups, all animals except one on day 8 in the 6H post-exposure group showed 1+ to 2+ pathology. Severity was less in the 6 h post-exposure group compared to the 24 h group.

All other organs tested (liver, kidney, heart and brain) showed no infection related pathology in any of the control or SAB-185 treated mice except one control mouse euthanized on day 6. The brain tissue from this animal showed 4+ changes on H&E staining including multifocal, perivascular mononuclear infiltrates affecting the hypothalamus. Mild multifocal vacuolation of neurons affecting the cerebrum and hypothalamus was also observed. Immunohistochemical staining was positive in the olfactory bulbs, cerebrum, hippocampus, thalamus and hypothalamus.

Detection of SAB-185 after IM Administration

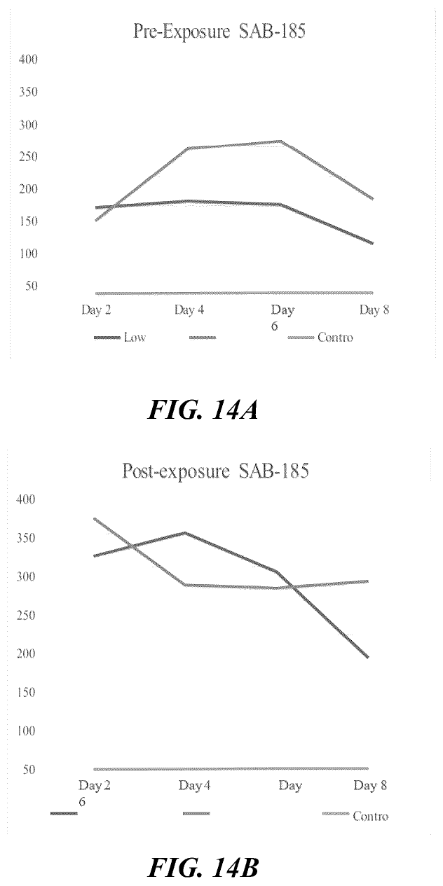

Sera from animals euthanized at the designated time periods were pooled and analyzed for the presence of total human anti-SARS-CoV-2 IgG using a commercially available quantitative ELISA. Any detectable human anti-SARS-CoV-2 IgG was assumed to be representative of SAB-185. FIGS. 14A-14B show that human anti-SARS-CoV-2 antibody was present in the mice treated pre-exposure with either low or high dose of SAB-185 (FIG. 14A) and in those treated 6 h and 24 h post-exposure (FIG. 14B). Anti-SARS-CoV-2 antibody was present in these animals at day 2 post challenge and persisted through day 8 post challenge. In contrast, no detectable antibody was present in control saline treated animals.

cPASS ELISA was utilized as a surrogate to assess anti-SARS-CoV-2 neutralization activity in serum from mice administered IM SAB-185. The 80% neutralizing antibody titer was taken to be the highest dilution resulting in greater than or equal to an 80% reduction in binding to recombinant RBD protein compared to kit negative control samples. It was evident that sera from all SAB-185 treated groups (pre-exposure low and high dose, post-exposure 6 h and 24 h) contained SARS-CoV-2 neutralizing antibody (80% neutralization titer 80-320) at all time points measured during the study. No detectable neutralizing antibody was found in control saline treated group at any point during the study (Table 9).

TABLE 9
SARS-CoV-2 neutralizing antibody
as measured by C-pass ELISA
80% neutralization titer (NT₈₀)

| | Day 2 | Day 4 | Day 6 | Day 8 |
|---|---|---|---|---|
| Saline control | 0 | 0 | 0 | 0 |
| low dose pre-exposure | 160 | 160 | 160 | 80 |
| high dose pre-exposure | 160 | 320 | 160 | 160 |
| 6 hour post-exposure | 320 | 320 | 320 | 160 |
| 24 hour post-exposure | 320 | 160 | 160 | 160 |

### Morbidity and Mortality Following Lethal Virus Challenge.

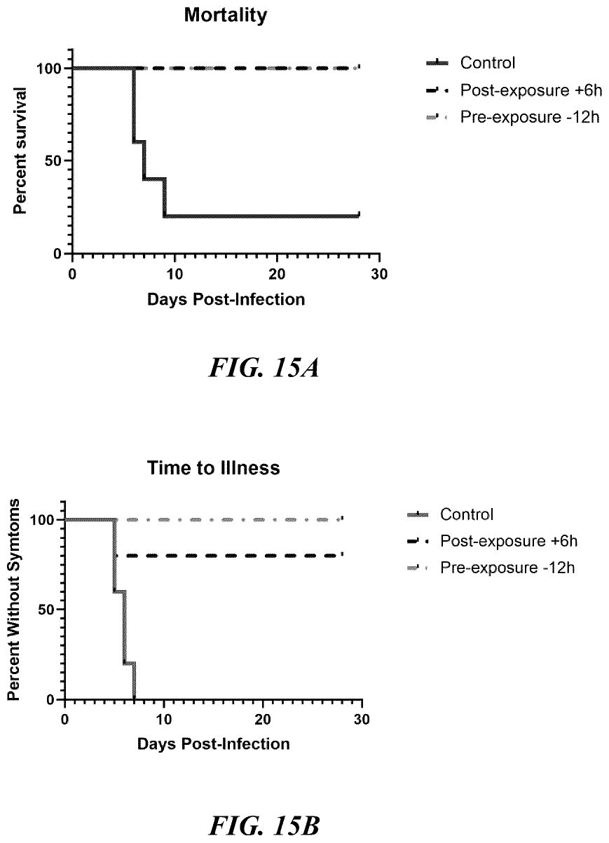

Three groups of five animals each were challenged with a lethal dose of SARS-CoV-2 intranasally and followed for 28 days, with daily observations including weight measurements and clinical signs of illness (Table 10). FIG. 15A shows a Kaplan Meier plot displaying the percent of mice surviving following challenge. In the control group, only one of five animals survived beyond day 9 post-challenge whereas all mice in the post-exposure and pre-exposure groups survived, demonstrating significant protection (p<0.003) against live SARS-CoV-2 challenge.

TABLE 10

| Clinical Observation | Score |
|---|---|
| Lethargy, ruffled fur, hunched | 1 |
| appearance, slow movement, | |
| increased body, temp, or 10% | |
| weight loss | |
| Decreased mobility, no longer | 2 |
| socializing with other animals, | |
| or 20%, weight loss | |
| Greatly labored respirations, gasping, | 3 |
| inability to move even after | |
| stimulation or 25% weight loss | |

The time to the development of signs of infection is shown in FIG. 15B. By day 7, all five animals in the control group demonstrated one or more signs of infection. In the mice treated with high dose SAB-185 six hours post-challenge, all but one, which developed signs of disease on day 5 (and later recovered), remained symptom free. Mice given high dose IM SAB-185 12 hours prior to intranasal challenge remained symptom free throughout the observation period.

### Discussion

The results of this study demonstrated that administering purified anti-SARS-CoV-2 hyperimmune polyclonal antibody product SAB-185 by IM injection provided significant protection against SARS-CoV-2 live virus challenge in K18 transgenic mice expressing the human ACE2 receptor. Low (1.88 mg) and high (3.75 mg) doses of SAB-185 polyclonal IgG antibody, when given pre-exposure 12 hours prior to intranasal challenge, significantly reduced SARS-CoV-2 infection of the lungs as evidenced by absence of or significantly reduced viral RNA detected by ddRT-PCR and by the inability to detect live virus by culture in lung tissue. Furthermore, histological examination and immunohistochemical staining of lung tissue demonstrated the absence of histopathology or infection in the lungs compared to untreated control mice.

Post-exposure animals subjected to high dose treatment with SAB-185 given IM six hours after intranasal SARS-CoV-2 challenge showed almost no lung pathology or infection as evidenced by the inability to recover live virus from the lungs of all 12 mice. High dose post-exposure treatment with SAB-185 administered 24 hours after challenge appeared to be less effective but still resulted in reduced SARS-CoV-2 viral RNA and a lower frequency of virus isolation from lung tissue compared to saline treated control mice. The trend for a greater reduction in viral load by administering SAB-185 six hours after challenge compared to 24 hours post-challenge may be explained by the earlier intervention to limit viral replication and systemic spread.

Human anti-SARS-CoV-2 IgG antibody was detected in pooled serum of mice given IM injections of SAB-185 before and after intranasal live virus challenge. The neutralizing activity of these antibodies was determined utilizing the cPASS assay. High levels of SARS-CoV-2 IgG and neutralizing antibodies in pooled serum samples demonstrated systemic dissemination of SAB-185 from the intramuscular injection site. In the pre-exposure groups, SARS-CoV-2 IgG and neutralizing antibody tended to be higher in the high dose group compared to the low dose group although the titers between groups at each time point were only within one dilution. For the post-exposure groups, both IgG and neutralizing antibody titers tended to be higher at days 2 and 4 in the mice dosed at six hours post-infection compared to those dosed at 24 hours but again the titers between groups were only within one dilution.

All mice demonstrated infection of the nasal turbinate following intranasal administration of live SARS-CoV-2. This was indicated by 2+ to 3+ histological changes upon examination of tissue samples by H&E and immunohistochemical staining. Without being bound by theory, the failure to prevent nasal turbinate infection was due to the lack of sufficient mucosal neutralizing antibody activity and the direct inoculation of high SARS-CoV-2 titers into the nasal passageway.

For mice in the untreated control group, except for one animal, evidence of SARS-CoV-2 infection was not observed in the brain, liver, kidney or heart by RT-PCR or viral culture. For the one animal, brain tissue showed evidence of infection by immunohistochemical staining at day 6 post-challenge. The detection of viral antigens in this lone animal indicates that CNS infection was an infrequent occurrence in our murine study. This contrasts the findings by Golden et al. JCI Insight 5(19):e142032 (2020) showing that most of the K18 transgenic mice in their study demonstrated evidence of brain involvement from 5-11 days post challenge.

In summary, IM SAB-185 administered pre- and post-SARS-CoV-2 challenge significantly reduced lung infection, morbidity and mortality in K18 mice compared to controls. This study provides preclinical evidence that IM SAB-185 could be an effective pre- and post-SARS-CoV-2 exposure treatment suitable to protect large populations such as medical and military personnel, immunosuppressed patients, critical infrastructure workers, and nursing home residents. These results support further clinical testing in humans to prevent COVID-19 disease.

### Materials and Methods

### Anti-SARS-CoV-2 Polyclonal Antibody SAB-185

SAB 185 was produced from transchromosomic cattle (Tc bovine) hyperimmunized in a prime-boost fashion with a DNA construct expressing full-length spike protein of the USA-WA1/2020 SARS-CoV-2 strain as the prime followed by boosts of recombinant ectodomain of spike protein. The prime consisted of two 12 mg doses of DNA formulated in SAB's proprietary adjuvant formulation (SAB-adj-1) given three weeks apart. The DNA vaccine was delivered by using the PharmaJet Stratis® IM injection device. The animals were subsequently boosted twice with 2 mg to 5 mg of recombinant ectodomain spike protein formulated in SAB-adj-1 at four week intervals. The cGMP lot of SAB-185 used in this experiment was pooled and purified from Tc bovine plasma obtained 8, 11 and 14 days after the second protein boost. The geometric mean 80% plaque reduction neutralization titer for this SAB-185 lot against a SARS-CoV-2 D614G strain was 4924.

### SARS-CoV-2 Challenge Virus

The live USA-WA1/2020 strain of SARS-CoV-2 was propagated in VERO81 cells and harvested at passage 6 to prepare virus stocks for use in challenge experiments. The virus stocks were stored at a concentration of 2×105 PFU/mL (lethal challenge) and 2×104 PFU/mL (sub-lethal challenge) as determined by plaque assay. The stocks were maintained in storage at −80° C.

### Prophylactic and Post-Exposure Intramuscular Administration of SAB-185

In the first experiment, five groups of K18-hACE2 transgenic mice (12 per group) were used. Groups 1 and 2 represented low and high dose pre-exposure treatment, respectively. The low dose consisted of 1.88 mg and the high dose 3.75 mg of SAB-185 in a total volume of 50 μl delivered IM 12 hours prior to intranasal challenge of SARS-CoV-2. Groups 3 and 4 represented post-exposure treatment and received high dose SAB-185 six hours and 24 hours, respectively, after intranasal SARS-CoV-2 live virus challenge. Animals in group 5 served as controls and received saline. All animals were infected intranasally with 103 PFU of SARS-CoV-2 in 50 μL on Day 0. On days 2, 4, 6 and 8, three animals from each group were euthanized and blood, lungs, nasal turbinate, brain, liver, kidney and heart removed at necropsy for histological examination. Portions of the lung were processed for the detection of live virus by culture and for detecting viral RNA by quantitative droplet digital RT-PCR (ddRT-PCR).

The second experiment examined the effectiveness of SAB-185 on reducing morbidity and mortality. Three groups of 5 animals each were challenged with lethal doses (1×104 pfu) of live SARS-CoV-2. Group 1 received high dose SAB-185 12 hours before challenge and group 2 received the same dose 6 hours after challenge. Animals in group 3 served as controls and received saline. Following intranasal challenge, the mice were observed for a total of 28 days for clinical symptoms of illness and mortality. Table 10 shows the symptom scale used to quantitate severity of clinical disease.

### Pre- and Post-Exposure Efficacy Analysis of IM SAB-185

In the first experiment, lungs were harvested at the indicated times. Half of the organ was fixed in formalin and processed for histological examination. Histological grading of H&E sections of the tissues was performed based on the grading scale in Table 11. The other half was homogenized in PBS and half of the homogenate processed to evaluate viral load by ddRT-PCR analysis. The primer sequences targeted the nucleocapsid genes of the USA-WA1/2020 virus strain. The forward and reverse primer sequences were 5′-GACCCCAAAATCAGCGAAAT-3′ (SEQ ID NO: 15) and 5′-TCTGGTTACTGCCAGTTGAATCTG-3′ (SEQ ID NO: 16), respectively. The probe sequence used was 5′-ACCCCGCATTACGTTTGGTGGACC-3′-3IABkFQ (SEQ ID NO: 17). The ddRT-PCR results were expressed as total number of RNA copies. The results from SAB 185 treated animals were compared to the untreated controls to assess the effect of SAB 185 on viral burden post-live virus challenge.

TABLE 11

| Histology Description | Severity | Score |
|---|---|---|
| 10% or less Immunoreactive Cells | Minimal | 1+ |
| 11%-25% Immunoreactive Cells | Mild | 2+ |
| 26%-50% Immunoreactive Cells | Moderate | 3+ |
| 51%-79% Immunoreactive Cells | Marked | 4+ |
| 80% or more Immunoreactive Cells | Severe | 5+ |

The other half of the homogenate was processed for recovery of live virus by tissue culture. Briefly aliquots of the homogenate were incubated in flasks containing confluent monolayers of VERO81 cells and incubated for 7 days at 37° C. Following incubation, the cells were scraped, clarified by centrifugation at 3000 rpm, and the cell pellet resuspended and applied to 24-well slides for immunofluorescence assay (IFA). The slides were fixed in Cytofix/Cytoperm and then examined for the presence of fluorescent infected cells using antibodies directed at the spike protein of SARS-CoV-2 (ProSci, Cat. No. 3525).

### Detection of SAB-185 Polyclonal Antibody in Mouse Serum

Total anti-SARS-CoV-2 IgG antibody was measured in mouse sera at the various time points using the COVID-SeroIndex quantitative assay. This assay detects human IgG and therefore was used to quantitate serum levels of SAB-185 following IM injection. The assay utilizes recombinant SARS-CoV-2 RBD antigen-coated plates that are reacted with mouse sera. Bound anti-RBD antibody is detected with an enzyme-linked anti-human IgG monoclonal antibody. For quantitation, positive samples are tested in a second orthogonal assay where plates coated with the SARS-CoV-2 spike protein are reacted with the sample, followed by bound antibody detection using the enzyme-linked anti-human IgG monoclonal antibody. The final quantitative readout of arbitrary units per milliliter (AU/mL) is calculated by comparing signal from the test samples to the calibration curve.

GeneScript cPASS technology was used to examine serum anti-SARS-CoV-2 neutralizing antibody activity in mice given SAB-185 by IM injection. This is an inhibition assay that detects total neutralizing antibodies in a specimen by mimicking the interaction between the SARS-CoV-2 virus and the host cell bearing the ACE-2 receptor protein. Briefly, plates coated with the ACE-2 protein are reacted with a cocktail containing the serum specimen of interest and recombinant receptor binding protein conjugated with HRP. If antibodies are present in the specimen that binds to the RBD protein in such a way as to interfere with binding to the ACE-2 protein, there will be an absence of color, indicating the presence of neutralizing antibodies. Because the assay does not require direct detection of bound antibody, the assay can be used to detect both human and animal anti-SARS-CoV-2 antibodies and is not isotype specific. For this assay, sera from the three animals euthanized on the indicated days were pooled and tested. Two-fold serial dilutions were made of the pooled sera, which was subsequently tested in the cPASS inhibition assay for surrogate neutralization activity as per manufacturer guidelines. The highest serum dilution resulting in 80% or greater inhibition was considered the endpoint neutralization titer.

### Data Analyses

Geometric mean RNA copy numbers for the indicated groups were calculated and compared for statistical significance by multiple means comparisons using one way ANOVA (Tukey Method). The results for the mortality and morbidity were analyzed by Log-rank (Mantel-Cox) test using the GraphPad Prism software package.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed herein is:

1. An ungulate-derived polyclonal human immunoglobulin composition, comprising a population of human immunoglobulins,
wherein the population of human immunoglobulins specifically binds a coronavirus protein selected from coronavirus S protein, coronavirus M protein, coronavirus E protein, or coronavirus Nucleocapsid protein, or a combination thereof.

2. A polyclonal human immunoglobulin composition, produced by immunizing a transgenic ungulate with a coronavirus protein or an antigenic fragment thereof, wherein the composition comprises a population of human immunoglobulins.

3. A polyclonal human immunoglobulin composition, comprising a population of human immunoglobulins,
wherein the population of human immunoglobulins specifically binds a coronavirus protein selected from coronavirus S protein, coronavirus M protein, coronavirus E protein, or coronavirus Nucleocapsid protein, or a combination thereof,
wherein the population of human immunoglobulins comprise glycans covalently linked to the human immunoglobulins, and
wherein the glycans comprise at least about 70%, at least about 80%, or at least about 90% N-Glycolylneuraminic acid (NGNA)-bearing glycans.

4. A method of making polyclonal human immunoglobulin for treatment of coronavirus disease, comprising administering an antigen comprising a coronavirus protein selected from coronavirus S protein, coronavirus M protein, coronavirus E protein, or coronavirus Nucleocapsid protein or an antigenic fragment thereof, or a polynucleotide encoding the antigen, to a transgenic ungulate, wherein the transgenic ungulate comprises a genome comprising a human immunoglobulin locus or an artificial chromosome comprising a human immunoglobulin locus, wherein the transgenic ungulate produces a population of human immunoglobulins that specifically binds the coronavirus protein selected from coronavirus S protein, coronavirus M protein, coronavirus E protein, or coronavirus Nucleocapsid protein, or a combination thereof.
